(19) United States
(12) Patent Application Publication
(10) Pub. No.: US 2026/0199460 A1
(43) Pub. Date: Jul. 16, 2026

(54) ANTI-IL-2 ANTIBODY COMBINATIONS AND METHODS OF USE THEREOF

(71) Applicant: Aulos Bioscience, Inc., Larkspur, CA (US)

(72) Inventors: Inbar AMIT, Modi'in (IL); Timothy WYANT, Bellingham, MA (US); Yanay OFRAN, Tel Aviv (IL); James Robert Vasselli, San Francisco, CA (US); Aron Marc Knickerbocker, Mill Valley, CA (US)

(73) Assignee: Aulos Bioscience, Inc., Larkspur, CA (US)

(21) Appl. No.: 19/381,211

(22) Filed: Nov. 6, 2025

Related U.S. Application Data
(63) Continuation-in-part of application No. 19/345,459, filed on Sep. 30, 2025.
Application No. 19/345,459 is a continuation-in-part of application No. 18/431,983, filed on Feb. 4, 2024.
Application No. 18/431,983 is a continuation-in-part of application No. 18/404,093, filed on Jan. 4, 2024.
Application No. 18/404,093 is a continuation-in-part of application No. PCT/US2023/079221, filed on Nov. 9, 2023.
(60) Provisional application No. 63/383,086, filed on Nov. 10, 2022.
(60) Provisional application No. 63/503,481, filed on May 21, 2023.
(60) Provisional application No. 63/503,977, filed on May 24, 2023.
(60) Provisional application No. 63/589,659, filed on Oct. 12, 2023.

(51) Int. Cl. A61K 39/395; A61P 35/00

(52) U.S. Cl. CPC A61K 39/3955; A61P 35/00

(57) ABSTRACT

Described herein are combination therapies comprising engineered anti-IL-2 antibodies in combination with an immune checkpoint inhibitor such as avelumab and an initial low dose of IL-2 and related therapeutic methods for a subjects with cancer, including a solid tumor and specifically non-small cell lung cancer (NSCLC).

### CROSS-REFERENCE TO RELATED APPLICATIONS

This Application is a Continuation-in-Part Application of U.S. application Ser. No. 19/345,459 filed Sep. 30, 2025, which is a Continuation-in-Part Application of U.S. application Ser. No. 18/431,983 filed Feb. 4, 2024, which is a Continuation-in-Part Application of U.S. application Ser. No. 18/404,093 filed Jan. 4, 2024, which is a Continuation-in-Part Application of PCT International Application No. PCT/US23/79221, International Filing Date Nov. 9, 2023, claiming the benefit of priority of U.S. Provisional Application No. 63/589,659 filed Oct. 12, 2023, U.S. Provisional Application No. 63/503,977 filed May 24, 2023, U.S. Provisional Application No. 63/503,481 filed May 21, 2023, and U.S. Provisional Application No. 63/383,086 filed Nov. 10, 2022, which are all hereby incorporated by reference in their entirety.

### SEQUENCE LISTING STATEMENT

The instant application contains a Sequence Listing which has been submitted electronically in XML format and is hereby incorporated by reference in its entirety. The XML formatted sequence listing, created on Oct. 28, 2025, is named P-621284-US5_SL_28OCT25.xml and is 11,360 bytes in size.

### FIELD OF THE INVENTION

The disclosure relates in general to the field of cancer treatment. In some embodiments, the present disclosure describes a combination therapy comprising engineered anti-IL-2 antibodies, low dose IL-2, and an immune checkpoint inhibitor such as avelumab and the use of the combination for treating cancers, including solid tumors and specifically non-small cell lung cancer (NSCLC).

### BACKGROUND

Interleukin-2 (IL-2), a potent T-cell-stimulating cytokine, was the first U.S. Food and Drug Administration (FDA)-approved immunotherapeutic effective in treating cancers such as metastatic melanoma and renal cell carcinoma (RCC). However, the need for high doses and frequent administration of IL-2 to induce therapeutic effects has led to severe side and off-target effects.

IL-2 signaling has two opposite effects. IL-2 can enhance immune response by activation of effector cells and induce their proliferation. Alternatively, IL-2 can tune down immune response by activation and proliferation of CD4+ regulatory T (Treg) cells. To facilitate these functions, IL-2 mediates its effect by binding to two forms of IL-2 receptor: i) trimeric receptors made up of IL-2Rα (CD25), IL-2Rβ (CD122), and a common IL-2Rγ (γc, CD132) chains, or ii) a dimeric receptor that consists of only the IL-2Rβ and IL-2Rγ subunits. Both the dimeric and trimeric receptors are able to transmit IL-2 binding signaling via the STAT5 pathway. However, IL-2 binds the αβγ receptor trimer at 100-fold tighter than the βγ receptor dimer. It has been demonstrated that the binding affinity of hIL-2 to the αβγ trimer is approximately 10 pM, whereas the hIL-2 affinity to the βγ dimer is 1 nM.

AU-007 is a human IgG1 monoclonal antibody (mAb) that binds to IL-2 with pM affinity on its CD25 binding epitope and completely inhibits its binding to CD25, without hindering its binding to CD132/CD122.

AU-007-bound IL-2 cannot bind trimeric (CD25, CD122, CD132) IL-2 receptors (IL-2R) on regulatory T cells (Tregs), vascular endothelium, or eosinophils, but IL-2's binding to dimeric (CD122, CD132) IL-2R on effector T (T eff) and NK cells is unhindered. AU-007 thus redirects IL-2 towards T eff and NK cell activation, while diminishing Treg activation and vascular leak, and redirects IL-2 generated from T eff cell expansion, converting a Treg-mediated autoinhibitory loop into an immune stimulating loop, driving tumor killing.

AU-007 bound IL-2 prolongs the T_{1/2} of IL-2, allowing endogenous IL-2 or low dose aldesleukin to initiate an anti-tumor response. AU-007 monotherapy at doses up to 12 mg/kg once every 2 weeks (Q2W) is safe and well tolerated, with initial signs of immune modulation consistent with AU-007's mechanism of action. More work is needed to ascertain how to optimize the benefits of treatment with AU-007 in combination with IL-2 in patients.

Blocking the programmed cell death-1 (PD-1)/programmed death ligand-1 (PD-L1) pathway through monoclonal antibodies (mAbs) is a proven therapeutic strategy against multiple solid tumors, including non-small cell lung cancer (NSCLC). Avelumab is a human IgG1 anti-PD-L1 mAb that is approved for use against several solid tumor histologies. In addition to blocking the PD-1/PD-L1 pathway, avelumab contains an active fragment crystallizable (Fc) moiety that triggers antibody-dependent cell-mediated cytotoxicity (ADCC), including NK cell mediated cytotoxicity, bringing both the innate and adaptive immune systems to bear against tumors.

Avelumab has been investigated for non-small cell lung cancer (NSCLC), showing antitumor activity in Phase 1 and 3 trials (JAVELIN Lung 100, JAVELIN Lung 200). While avelumab was not superior to chemotherapy in improving overall survival (OS) in the first-line (JAVELIN Lung 100) or second-line (JAVELIN Lung 200) settings, it demonstrated potential in certain patient groups, particularly those with high PD-L1 expression. While avelumab has shown promising antitumor activity and an acceptable safety profile in NSCLC, its efficacy as a standalone first-line or second-line treatment has not consistently met primary survival endpoints compared to standard chemotherapy. Therefore, more work is needed to ascertain how to optimize the benefits of treatment with avelumab as well.

### SUMMARY

In one aspect, disclosed herein is a combination therapy comprising an anti-IL-2 antibody or a pharmaceutical composition thereof, a loading dose of a low dose of IL-2 or a pharmaceutical composition thereof, and avelumab or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO: 2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5; wherein said anti-IL-2 antibody is formulated for administration at a dose of 9 mg/kg of a subject's body weight, the loading low dose of IL-2 is formulated for subcutaneous injection and administration at a dose of 135,000 IU/kg of a subject's body weight, and said avelumab is formulated for administration at a dose of 800 mg wherein said pharmaceutical composition further comprises a pharmaceutically acceptable carrier. In a related aspect, this combination therapy is for treating unresectable locally advanced or metastatic non-small-cell lung cancer (NSCLC) that is PD-L1 positive. In a further related aspect, this combination therapy is for 2^{nd}- or 3^{rd}-line treatment.

In another related aspect of the combination therapy, the amino acid sequence of the VH comprises the amino acid sequence of SEQ ID NO:6 and the amino acid sequence of VL comprises the amino acid sequence of SEQ ID NO:7. In a further related aspect, the amino acid sequence of the full length heavy chain is set forth in SEQ ID NO:8 and the amino acid sequence of the full length light chain is set forth in SEQ ID NO:9.

In another related aspect of the combination therapy, the antibody comprises an IgG, IgA, IgM, IgE, IgD, a Fv, a scFv, a Fab, a F(ab′)2, a minibody, a diabody, or a triabody. In a further related aspect, the antibody comprises a heavy chain comprising a mutation that reduces binding to fragment crystallizable gamma receptors (FcγRs), wherein reduced binding is compared with said antibody lacking the mutation in the heavy chain that affects FcγRs binding. In yet another further related aspect, the mutation comprises L234A, L235A (LALA) mutations.

In another aspect, disclosed herein is a method of treating an unresectable locally advanced or metastatic non-small-cell lung cancer (NSCLC) that is PD-L1 positive in a subject, said method comprising administering to said subject a combination therapy comprising an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5, wherein said pharmaceutical composition(s) further comprises a pharmaceutically acceptable carrier, and wherein said treating comprises second-line or third-line treatment, thereby treating said unresectable locally advanced or metastatic NSCLC that is PD-L1 positive NSCLC in said subject.

In a related aspect of a method of treating an unresectable locally advanced or metastatic NSCLC, the administration of said loading dose of IL-2 is prior to, concurrent with, or following the administration of said anti-IL-2 antibody, said avelumab, or both.

In another related aspect of method of treating an unresectable locally advanced or metastatic NSCLC, the method further comprises the step of administering one or more additional doses of said anti-IL-2 antibody or a pharmaceutical composition thereof, wherein said pharmaceutical composition further comprises a pharmaceutically acceptable carrier.

In another related aspect of method of treating an unresectable locally advanced or metastatic NSCLC, the method comprises administering additional doses of said anti-IL-2 antibody or a pharmaceutical composition thereof are administered to said subject once every two weeks. In yet another related aspect of method of treating an unresectable locally advanced or metastatic NSCLC, the method comprises the step of administering one or more additional doses of said avelumab or a pharmaceutical composition thereof, wherein said pharmaceutical composition further comprises a pharmaceutically acceptable carrier. In a further related aspect, the one or more additional doses of said avelumab or a pharmaceutical composition thereof are administered to said subject once every two weeks.

In another related aspect of method of treating an unresectable locally advanced or metastatic NSCLC, the method further comprises the step of administering one or more booster doses of IL-2 or a pharmaceutical composition thereof, wherein said pharmaceutical composition further comprises a pharmaceutically acceptable carrier. In a further related aspect, the administration of said one or more booster doses of IL-2 is prior to, concurrent with, or following the administration of said one or more additional doses of said anti-IL-2 antibody, one or more additional doses of said avelumab, or both. In another further related aspect, at least one of the one or more IL-2 booster doses is administered at a dose of 135,000 IU/kg of said subject's body weight. In yet another further related aspect, the at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

In another related aspect of method of treating an unresectable locally advanced or metastatic NSCLC, the method comprises (i) reducing the size of the tumor, (ii) inhibiting or reducing growth of the tumor, (iii) inhibiting or reducing metastases of said tumor, (iv) inhibiting the production of new lesions, (v) decreasing or shrinking target lesions, (vi) eliminating target lesions, or (vii) any combination thereof. In a further related aspect, said NSCLC progressed after prior checkpoint inhibitor therapy in said subject. In yet another further related aspect, said NSCLC comprises squamous NSCLC. In other further related aspects, said NSCLC comprises non-squamous NSCLC.

### BRIEF DESCRIPTION OF THE DRAWINGS

The present disclosure of engineered anti-IL-2 antibody and combination therapies comprising the anti-IL-2 antibody, both as to their generation and method of use, together with objects, features, and advantages thereof, may best be understood by reference to the following detailed description when read with the accompanying drawings in which:

FIG. 1 graphically presents the administration and dosage schemes of AU-007 once every 2 weeks (Q2W) monotherapy (left-1A), AU-007+a single IL-2 Loading Dose (center-1B), and combination therapy of AU-007 Q2W+IL-2 Q2W (right-1C). When administered, recombinant human IL-2 (aldesleukin) was administered subcutaneously, at much lower doses and much less frequently than the approved regimen of intravenously administered aldesleukin. The terms “3+3” and “1+2” refer to the size of the cohort at a given dose level. For 3+3, the first 3 enrolled patients were administered AU-007 at the dose level that is noted. If no dose-limiting toxicities (DLTs) were seen, then administration was escalated to the next highest dose level. However, if any of the first 3 patients had a dose-limiting toxicity that was drug-related, then an additional three more patients would have received treatment at that same dose level to see if they have DLTs before escalating. 1+2 follows the same principle—start with one patient, if DLTs were observed, add two more, or if no DLTs were observed in the first single patient, then dosages can be escalated.

FIG. 2 provides a timeline of the treatment with AU-007 in combination with single-dose aldesleukin and avelumab and optional aldesleukin boost administration and tumor assessment. Cycles are 8 weeks (56 days). AU-007 and avelumab are administered 4 times in a cycle (Day 1, 15, 29, 43—see black arrow and black rectangle, respectively); Aldesleukin is administered as a loading dose once in Cycle 1 (Day 1—see ellipse). Tumor evaluation (black diamond) using Response Evaluation Criteria In Solid Tumors (RECIST) evaluation of CT scan, MRI, PET scan, or ultrasound to measure existing tumor size and assess for any new tumors is carried out at the end of each cycle. Biopsy (4-point star) is conducted to evaluate immune-modulating effect of drug within tumor by evaluating immune cell infiltration. The Dose-Limiting Toxicity (DLT) period lasts 4 weeks and informs dose escalation decisions. The aldesleukin boost administration (not shown) is administered in patients who are tolerating treatment and are clinically stable at the discretion of the Investigator and Sponsor based on the following: (1) with each cycle (Q8W, Day 1 of cycle) until objective tumor shrinkage is observed on radiologic imaging or physical exam or (2) with objective signs of worsening tumor growth kinetics: e.g., previously shrinking tumors becoming stable, increase in tumor markers, tumor growth, or appearance of new tumor growth in a tumor that had previously been stable or had decreased in size, or any combination thereof. If a patient continues to subsequent cycles, they begin with AU-007 and avelumab on Cycle 2 Day 1, then AU-007 administered with avelumab on Days 15, 29, and 43 of Cycle 2; and so on.

FIGS. 3A and 3B show early evidence of response of NSCLC patients to administration of AU-007+IL-2 loading dose+avelumab. FIG. 3A presents the best response in NSCLC patients treated with Imneskibart (AU-007)+IL-2+avelumab. FIG. 3B presents the percentage change over time versus baseline of target lesion sum of diameters in NSCLC patients treated with Imneskibart (AU-007)+IL-2+avelumab.

FIGS. 4A and 4B present CT scans showing 43% tumor reduction at 8 weeks in the pulmonary target lesions in a NSCLC patient who had previously progressed through prior anti-PD-1+chemotherapy. Patient was treating with RP2D Imneskibart (AU-007)+IL-2+avelumab. FIG. 4A is the baseline CT scan. FIG. 4B is the CT scan at 8-weeks (end of cycle 1) showing a 43% decrease in target lesion size.

### DETAILED DESCRIPTION

In the following detailed description, numerous specific details are set forth in order to provide a thorough understanding of the antibodies disclosed herein. However, it will be understood by those skilled in the art that preparation and uses of antibodies disclosed herein may in certain cases be practiced without these specific details. In other instances, well-known methods, procedures, and components have not been described in detail so as not to obscure the disclosure presented herein.

Throughout this application, various references or publications are cited. Disclosures of these references or publications in their entireties are hereby incorporated by reference into this application in order to more fully describe the state of the art to which this invention pertains.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen.

By “specifically binding” is meant that the binding is selective for the antigen of interest and can be discriminated from unwanted or nonspecific interactions. For example, an antibody is said to specifically bind an IL-2 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a nanobody, minibodies, diabodies, triabodies, tetrabodies, and single domain antibodies (see, e.g., Hudson and Souriau, Nature Med. 9:129-134 (2003)). Also encompassed are humanized, primatized, and chimeric antibodies as these terms are generally understood in the art.

A skilled artisan would appreciate that in certain embodiments, the term “anti-IL-2 antibody” as used herein is interchangeable with the term “anti-human-IL-2 antibody”, having all the same qualities and meanings. Similarly, as used throughout, in certain embodiments, the term “IL-2” is interchangeable with the term “human IL-2”, having all the same qualities and meanings.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1”, “CDR2”, and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In some embodiments, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat E A, Wu T T, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: Firstly, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Secondly, they devised a peptide sequence numbering system in which homologous immunoglobulin residues were assigned the same position number. One skilled in the art can unambiguously assign to any variable domain sequence what is now commonly called Kabat numbering without reliance on any experimental data beyond the sequence itself. Thirdly, Kabat and Wu calculated variability for each Kabat-numbered sequence position, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

Chothia and coworkers (Cyrus Chothia, Arthur M. Lesk. Canonical structures for the hypervariable regions of immunoglobulins. Journal of Molecular Biology, 196, 4, 8 (1987)) found that certain sub portions within Kabat CDRs adopt nearly identical peptide backbone conformations, despite having great diversity at the level of amino acid sequence. These sub portions were designated as L1, L2 and L3 or H1, H2 and H3, where the “L” and the “H” designates the light chain and the heavy chains regions, respectively. These regions may be referred to as Chothia CDRs, which have boundaries that overlap with Kabat CDRs.

More recent studies have shown that virtually all antibody binding residues fall within regions of structural consensus (Kunik, V. et al., PloS Computational Biology 8 (2): el002388 (February 2012)). In some embodiments, these regions are referred to as antibody binding regions. It was shown that these regions can be identified from the antibody sequence as well. “Paratome”, an implementation of a structural approach for the identification of structural consensus in antibodies, was used for this purpose. (Ofran, Y. et al., J. Immunol. 757:6230-6235 (2008)). While residues identified by Paratome cover virtually all the antibody binding sites, the CDRs (as identified by the commonly used CDR identification tools) miss significant portions of them. Antibody binding residues which were identified by Paratome but were not identified by any of the common CDR identification methods are referred to as Paratome-unique residues. Similarly, antibody binding residues that are identified by any of the common CDR identification methods but are not identified by Paratome are referred to as CDR-unique residues. Paratome-unique residues make crucial energetic contributions to antibody-antigen interactions, while CDRs-unique residues make a rather minor contribution. These results allow for better identification of antigen binding sites.

IMGT® is the international ImMunoGeneTics information System®, (See, Nucleic Acids Res. 2015 January; 43 (Database issue): D413-22. doi: 10.1093/nar/gku1056. Epub 2014 Nov. 5 Free article. PMID: 25378316 LIGM: 441 and Dev Comp Immunol. 2003 January; 27 (1): 55-77). IMGT is a unique numbering system for immunoglobulin and T cell receptor variable domains and Ig superfamily V-like domains (Lefranc et al., Dev Comp Immunol. 27:55-77 (2003)). IMGT® presents a uniform numbering system for these IG and TcR variable domain sequences, based on aligning 5 or more IG and TcR variable region sequences, taking into account and combining the Kabat definition of FRs and CDRs, structural data, and Chothia's characterization of the hypervariable loops. IMGT is considered well known in the art as a universal numbering scheme for antibodies.

In some embodiments, identification of potential variant amino acid positions in the VH and VL domains uses the IMGT system of analysis. In some embodiments, identification of potential variant amino acid positions in the VH and VL domains uses the Paratome system of analysis. In some embodiments, identification of potential variant amino acid positions in the VH and VL domains uses the Kabat system of analysis. In some embodiments, identification of potential variant amino acid positions in the VH and VL domains uses the Clothia system of analysis.

In describing variant amino acid positions present in the VH and VL domains, in some embodiments the IMGT numbering is used. In describing variant amino acid positions present in the VH and VL domains, in some embodiments the Paratome numbering is used. In describing variant amino acid positions present in the VH and VL domains, in some embodiments the Kabat numbering is used. In describing variant amino acid positions present in the VH and VL domains, in some embodiments the Clothia numbering is used.

Antigen binding sequences are conventionally located within the heavy chain and light chain variable regions of an antibody. These heavy and light chain variable regions may, in certain instances, be manipulated to create new binding sites, for example to create antibodies or fragments thereof, that bind to a different antigen or to a different epitope of the same antigen. In some embodiments, as described herein, manipulating the sequences of a heavy chain variable region or the sequences of a light chain variable region, or both, would create a new binding site for a second antigen.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

### Methods of Use

In some embodiments, the present disclosure provides methods of treating a cancer in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the present disclosure provides methods of treating a solid tumor.

In some embodiments, the present disclosure provides methods of treating an unresectable locally advanced or metastatic solid cancer in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof.

In some embodiments, the present disclosure provides methods of treating an unresectable locally advanced or metastatic non-small cell lung cancer (NSCLC) in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the NSCLC is PD-L1 positive. In some embodiments, the NSCLC is PD-1 positive. In some embodiments, the NSCLC progressed after prior checkpoint inhibitor therapy in the subject. In some embodiments, the NSCLC comprises squamous NSCLC. In some embodiments, the NSCLC comprises non-squamous NSCLC.

In some embodiments, the present disclosure provides methods of treating an unresectable locally advanced or metastatic non-small cell lung cancer (NSCLC) that is PD-L1 positive, in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the present disclosure provides methods of treating an unresectable locally advanced or metastatic non-small cell lung cancer (NSCLC) that is PD-1 positive, in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the NSCLC progressed after prior checkpoint inhibitor therapy in the subject. In some embodiments, the NSCLC comprises squamous NSCLC. In some embodiments, the NSCLC comprises non-squamous NSCLC.

In certain embodiments, the target population for treatment with the methods disclosed herein is patients with unresectable locally advanced PD-L1-positive (tumor proportion score [TPS]≥1%) or metastatic PD-L1-positive (tumor proportion score [TPS]≥1%) NSCLC that does not contain an EGFR or ALK mutation. In other embodiments, the target population for treatment with the methods disclosed herein is patients with unresectable locally advanced PD-L1-positive (tumor proportion score [TPS]≥50%) or metastatic PD-L1-positive (tumor proportion score [TPS]≥50%) NSCLC that does not contain an EGFR or ALK mutation, if 0 of the first 10 patients did not see sufficient efficacy.

In some embodiments, the present disclosure provides methods of treating melanoma, renal cell carcinoma, non-small cell lung cancer or other cancer conditions.

In some embodiments, the present disclosure provides methods of treating a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In other embodiments, the present disclosure provides methods of treating or preventing a condition caused by IL-2 binding to endothelial CD25 expressing cells, e.g., pulmonary edema, or IL-2-induced vascular leakage.

In other embodiments, the present disclosure provides methods of treating a solid tumor. In some embodiments, a solid tumor comprises a head and neck cancer, a head and neck squamous cell carcinoma (HNSCC), a pancreatic cancer, a lung cancer, a thyroid cancer, a non-small cell lung cancer (NSCLC), a nasopharyngeal carcinoma, a melanoma, an acral melanoma, a uveal melanoma, a colorectal cancer (CRC), a bladder cancer, cholangiocarcinoma (bile duct cancer), a uterine cancer, a cervical cancer, a gallbladder cancer, a cutaneious squamous carcinoma, or a renal cell carcinoma (RCC). In some embodiments, a solid tumor comprises a head and neck cancer, a pancreatic cancer, or a non-small cell lung cancer. In some embodiments, a solid tumor comprises a non-small cell lung cancer (NSCLC), a melanoma, a metastatic melanoma, a primary melanoma and a metastatic melanoma, or a renal cell carcinoma (RCC). In some embodiments, a solid tumor comprises a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma.

In some embodiments, a solid tumor comprises a head and neck cancer. In some embodiments, a solid tumor comprises a pancreatic cancer. In some embodiments, a solid tumor comprises a lung cancer. In some embodiments, a solid tumor comprises a thyroid cancer. In some embodiments, a solid tumor comprises a nasopharyngeal carcinoma. In some embodiments, a solid tumor comprises a melanoma. In some embodiments, a melanoma comprises an acral melanoma or a uveal melanoma. In some embodiments, a solid tumor comprises a colorectal cancer (CRC). In some embodiments, a solid tumor comprises a bladder cancer. In some embodiments, a solid tumor comprises cholangiocarcinoma (bile duct cancer). In some embodiments, a solid tumor comprises a uterine cancer. In some embodiments, a solid tumor comprises a cervical cancer. In some embodiments, a solid tumor comprises a gallbladder cancer. In some embodiments, a solid tumor comprises a renal cell carcinoma (RCC). In some embodiments, a head and neck cancer is a head and neck squamous carcinoma (HNSCC1). In some embodiments, a CRC has high MSI. In some embodiments, a melanoma has a wild-type BRAF gene. In some embodiments, a melanoma has a mutant BRAF gene. In some embodiments, a pancreatic cancer is an adenocarcinoma. In some embodiments, a solid tumor comprises a non-small cell lung cancer (NSCLC). In some embodiments, a NSCLC is a squamous cancer. In some embodiments, a NSCLC is a non-squamous cancer. In some embodiments, a NSCLC has a mutated epidermal growth factor receptor (EGFRm). In some embodiments, a solid tumor comprises a cutaneous squamous carcinoma.

In some embodiments, the present disclosure provides methods of treating an NSCLC in a subject. In some embodiments, the present disclosure provides a method of treating an unresectable locally advanced or metastatic NSCLC. In some embodiments, a method of treating an unresectable locally advanced or metastatic NSCLC comprises a NSCLC that is PD-L1 positive. In some embodiments, a method of treating an unresectable locally advanced or metastatic NSCLC comprises a NSCLC that is PD-1 positive. In some embodiments, a method of treating an unresectable locally advanced or metastatic NSCLC comprises a NSCLC that is PD-L1 positive in a subject, wherein said treating comprises second-line or third-line treatment. In some embodiments, a method of treating an unresectable locally advanced or metastatic NSCLC comprises a NSCLC that is PD-1 positive in a subject, wherein said treating comprises second-line or third-line treatment. In some embodiments, a method of treating an unresectable locally advanced or metastatic NSCLC comprises a squamous or a non-squamous NSCLC that is PD-L1 positive. In some embodiments, a method of treating an unresectable locally advanced or metastatic NSCLC comprises a squamous or a non-squamous NSCLC that is PD-1 positive.

In some embodiments, the method described herein comprises treating the primary cancer and secondary metastasis of the cancer. In some embodiments, the method described herein comprises treating the secondary metastasis of the cancer. In some embodiments, the method described herein is a first line treatment of the cancer. In some embodiments, the method described herein is a first line treatment of the cancer or a later line of therapy. In some embodiments, the method described herein is a second line treatment of the cancer. In some embodiments, the method described herein is a third line treatment of the cancer. In some embodiments, the method described herein is a fourth line treatment of the cancer. In some embodiments, the method described herein is a fifth line treatment of the cancer. In some embodiments, the method described herein is a sixth line treatment of the cancer. In some embodiments, the method described herein is a seventh line treatment of the cancer. In some embodiments, the method described herein is an eighth line treatment of the cancer. In some embodiments, the method described herein is a ninth line treatment of the cancer. In some embodiments, the method described herein comprises a second line treatment or a third line treatment of the cancer.

In some embodiments, the cancer comprises an unresectable locally advanced or metastatic cancer. In other embodiments, the cancer comprises an unresectable locally advanced or metastatic solid cancer. In some embodiments, the unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

In other embodiments, the cancer comprises an unresectable locally advanced or metastatic NSCLC. In other embodiments, the unresectable locally advanced or metastatic NSCLC comprises a PD-L1-positive NSCLC. In some embodiments, the unresectable locally advanced or metastatic NSCLC comprises squamous or non-squamous that is PD-L1-positive. In some embodiments, the tumor proportion score [TPS] of the NSCLC is greater than or equal to 1%. In some embodiments, the NSCLC does not harbor an activating EGFR mutation. In some embodiments, the NSCLC does not harbor an ALK rearrangement. In some embodiments the NSCLC does not harbor an activating EGFR mutation that has progressed during or following treatment with an anti-PDx (either PD-1 or PD-L1). In some embodiments the NSCLC does not harbor an ALK rearrangement that has progressed during or following treatment with an anti-PDx (either PD-1 or PD-L1). In some embodiments, the NSCLC progressed during or following treatment with an anti-PDx (either PD-1 or PD-L1) with platinum-based chemotherapy. In other embodiments, the NSCLC progressed during or following treatment with an anti-PDx (either PD-1 or PD-L1) without platinum-based chemotherapy.

In some embodiments, treating an unresectable locally advanced or metastatic NSCLC comprises treating the primary NSCLC and secondary metastasis of the NSCLC. In some embodiments, treating an unresectable locally advanced or metastatic NSCLC comprises treating the secondary metastasis of the NSCLC. In some embodiments, treating an unresectable locally advanced or metastatic NSCLC comprises a first line treatment of the NSCLC. In some embodiments, treating an unresectable locally advanced or metastatic NSCLC comprises a second line treatment of the NSCLC. In some embodiments, treating an unresectable locally advanced or metastatic NSCLC comprises a third line treatment of the NSCLC. In some embodiments, treating an unresectable locally advanced or metastatic NSCLC comprises a fourth line treatment of the NSCLC. In some embodiments, treating an unresectable locally advanced or metastatic NSCLC comprises a fifth line treatment of the NSCLC. In some embodiments, treating an unresectable locally advanced or metastatic NSCLC comprises a sixth line treatment of the NSCLC. In some embodiments, treating an unresectable locally advanced or metastatic NSCLC comprises a seventh line treatment of the NSCLC. In some embodiments, treating an unresectable locally advanced or metastatic NSCLC comprises an eighth line treatment of the NSCLC. In some embodiments, treating an unresectable locally advanced or metastatic NSCLC comprises a ninth line treatment of the NSCLC. In some embodiments, treating an unresectable locally advanced or metastatic NSCLC comprises second and third line treatments of the NSCLC.

In some embodiments, a “first line” treatment is the initial and preferred therapeutic option for a given medical condition or disease, chosen because it is considered the most effective and least risky based on evidence and clinical guidelines. In some embodiments, a “second line” treatment is a treatment that is given to a patient when the initial treatment (first-line therapy) is not effective or stops working. In some embodiments, a “third line” treatment is a treatment that is given to a patient when both initial treatment (first-line therapy) and subsequent treatment (second-line therapy) are not effective or stop working. Fourth line, fifth line, etc. treatments are similarly treatment that is given to a patient when prior treatments failed, e.g., were not effective or stopped working.

In some embodiments, the present disclosure provides a method of treating an NSCLC in a subject comprising administering to said subject an anti-IL-2 antibody or a pharmaceutical composition thereof and avelumab or a pharmaceutical composition thereof. In some embodiments, the present disclosure provides a method of treating an NSCLC in a subject comprising administering to said subject an anti-IL-2 antibody or a pharmaceutical composition thereof, a loading dose of low dose IL-2 or a pharmaceutical composition thereof, and avelumab or a pharmaceutical composition thereof.

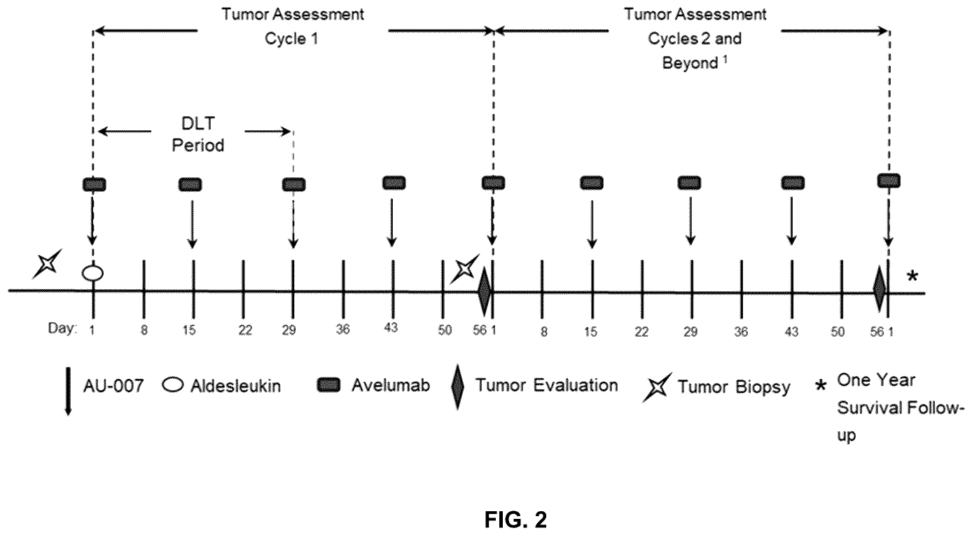

In some embodiments, multiple doses of anti-IL-2 antibody are administered (FIG. 2). In some embodiments, 2-50 doses of anti-IL-2 antibody are administered. In other embodiments, 20-50 doses of anti-IL-2 antibody are administered. In other embodiments, 10-40 doses of anti-IL-2 antibody are administered. In other embodiments, 20-30 doses of anti-IL-2 antibody are administered. In other embodiments, more than 20 doses of anti-IL-2 antibody are administered. In other embodiments, more than 30 doses of anti-IL-2 antibody are administered. In other embodiments, more than 40 doses of anti-IL-2 antibody are administered. In other embodiments, more than 50 doses of anti-IL-2 antibody are administered. In some embodiments, anti-IL-2 antibody doses are administered at regular intervals throughout the treatment. In some embodiments, the interval between anti-IL-2 antibody doses is once every 2 weeks, 4 weeks, 6 weeks, or 8 weeks. In some embodiments, the interval between anti-IL-2 antibody doses is 2 weeks.

In some embodiments, a single dose of avelumab is administered (FIG. 2). In some embodiments, multiple doses of avelumab are administered. In some embodiments, 2-50 doses of avelumab are administered. In other embodiments, 20-50 doses of avelumab are administered. In other embodiments, 10-40 doses of avelumab are administered. In other embodiments, 20-30 doses of avelumab are administered. In other embodiments, more than 20 doses of avelumab are administered. In other embodiments, more than 30 doses of avelumab are administered. In other embodiments, more than 40 doses of avelumab are administered. In other embodiments, more than 50 doses of avelumab are administered. In some embodiments, avelumab doses are administered at regular intervals throughout the treatment. In some embodiments, the interval between avelumab doses is once every 2 weeks, 4 weeks, 6 weeks, or 8 weeks. In some embodiments, the interval between avelumab doses is 2 weeks.

In some embodiments, a method as described herein comprises administering a loading dose of a low dose of IL-2 or a pharmaceutical composition thereof to the subject (FIG. 2). In some embodiments, the method comprises further administering one or more booster doses of a low dose of IL-2 or a pharmaceutical composition thereof to the subject. In some embodiments, the method comprises further administering a single booster dose of a low dose of IL-2 or a pharmaceutical composition thereof to the subject. In other embodiments, the method comprises further administering two or more booster doses of a low dose of IL-2 or a pharmaceutical composition thereof to the subject. In other embodiments, the method comprises further administering three or more booster doses of a low dose of IL-2 or a pharmaceutical composition thereof to the subject. In other embodiments, the method comprises further administering four or more booster doses of a low dose of IL-2 or a pharmaceutical composition thereof to the subject. In other embodiments, the method comprises further administering five or more booster doses of a low dose of IL-2 or a pharmaceutical composition thereof to the subject. In other embodiments, the method comprises administering a loading dose of low dose IL-2 and two, three, four, five, or six booster doses of low dose IL-2. In other embodiments, the method comprises administering a loading dose of low dose IL-2 and 6-10 or 10 or more booster doses of low dose IL-2. In some embodiments, low dose IL-2 boosters are administered at regular intervals throughout the treatment. In some embodiments, the interval between low dose IL-2 boosters is once every 2 weeks, 4 weeks, 6 weeks, or 8 weeks. In some embodiments, the interval between low dose IL-2 boosters is 2 weeks. In some embodiments, the interval between low dose IL-2 boosters is 8 weeks.

A skilled artisan would appreciate that duration of a treatment therapy, for example but not limited to for treating an unresectable locally advanced or metastatic NSCLC, may be based on the response or lack of response by the cancer to the treatment. In some embodiments, duration of a method of treating an unresectable locally advanced or metastatic NSCLC comprises about 2 years for patients who are receiving benefit from the treatment therapy. Benefit may in some embodiments be measured by tumor stabilization, tumor shrinkage, inhibiting or reducing growth of the tumor, inhibiting or reducing metastases of the tumor, inhibiting the production of new lesions, elimination of lesions, or any combination thereof. In some embodiments, duration of a method of treating an unresectable locally advanced or metastatic NSCLC comprises less than 2 years for patients who are receiving benefit from the treatment therapy. In some embodiments, duration of a method of treating an unresectable locally advanced or metastatic NSCLC comprises 1 year for patients who are receiving benefit from the treatment therapy. In some embodiments, duration of a method of treating an unresectable locally advanced or metastatic NSCLC comprises less than 1 years for patients who are receiving benefit from the treatment therapy.

In some embodiments of a method of treatment disclosed herein using a combination therapy, the administration of each components may be on a different treatment schedule for different durations (FIG. 2). For example but not limited to administration of IL-2 booster doses, wherein the number of booster doses administered may vary widely between patients. In some embodiments, a patient receives at least one additional IL-2 dose (a “booster” dose). In some embodiments, a patient receives at least 1, 2, 3, 4, 5, 6, 7, 8, 9, 10, 11, or 12 additional IL-2 doses (“booster” doses) over a 2 year period. In some embodiments, a patient receives less than 12 booster IL-2 doses over a 2 year period. In some embodiments, a patient receives 6 booster IL-2 doses over a 1 year period. In some embodiments, a patient receives less than 6 booster IL-2 doses over a 1 year period. In some embodiments, a patient receives 1, 2, 3, 4, 5, or 6 booster IL-2 doses over a 1 year period. In some embodiments, a patient receives 3 booster IL-2 doses over a 6 month period. In some embodiments, a patient receives less than 3 booster IL-2 doses over a 6 month period. In some embodiments, a patient receives 1, 2, or 3 booster IL-2 doses over a 6 month period.

In some embodiments, the IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO:5.

In some embodiments, the loading dose of IL-2 is subcutaneously administered. In some embodiments, the booster dose of IL-2 is subcutaneously administered. In some embodiments, the loading dose of IL-2 is administered at a dose of between about 15,000 IU/kg of said subject's body weight-500,000 IU/kg of said subject's body weight. In some embodiments, the booster dose of IL-2 is administered at a dose of between about 15,000 IU/kg of said subject's body weight-500,000 IU/kg of said subject's body weight. In some embodiments, the booster dose of IL-2 is administered at a dose of 135,000 IU/kg of said subject's body weight.

As used herein, the terms “subcutaneous”, “SC”, and “SQ”, and the like, may be used interchangeably having all the same meanings and qualities.

In some embodiments of the methods described herein that comprise administering a booster dose of IL-2, the booster dose of IL-2 is administered to the subject with each treatment cycle, e.g., until objective tumor shrinkage, inhibiting or reducing growth of the tumor, inhibiting or reducing metastases of the tumor, inhibiting the production of new lesions, elimination of lesions, or any combination thereof, is observed on radiologic imaging or physical exam. In some embodiments, objective tumor shrinkage is observed on radiologic imaging, physical exam, or a combination thereof. In other embodiments, the booster dose of IL-2 is administered to the subject when the subject has one or more objective signs of worsening tumor growth kinetics. In some embodiments, a sign of worsening tumor growth kinetics comprises previously shrinking tumors becoming stable. In other embodiments, a sign of worsening tumor growth kinetics comprises new tumor growth. In other embodiments, a sign of worsening tumor growth kinetics comprises appearance of new tumor growth in a tumor that was previously stable or had decreased in size. In other embodiments, a sign of worsening tumor growth kinetics comprises an increase in tumor markers. In other embodiments, a sign of worsening tumor growth kinetics comprises appearance of metastases. In other embodiments, a sign of worsening tumor growth kinetics comprises any combination of the signs described herein. In some embodiments, a treatment cycle is 8 weeks (FIG. 2). In other embodiments, a treatment cycle is 2, 4, 6, 10, or 12 weeks.

A skilled artisan would appreciate that a “Target Lesion” is a measurable (measurable defined per the RECIST (Response Evaluation Criteria in Solid Tumors) rules) tumor lesion (locally advanced or metastatic) that's measurement is added to the measurements of up to 4 other Target Lesions and the total combined diameters of the Target Lesions is used to follow if the tumor is increasing or decreasing in size after each cycle of treatment. In certain embodiments, methods described herein inhibit or reduce metastases. This may be observed by the absence of new lesions. In some embodiments, methods described herein result in decrease or shrinkage in a target lesion. This may be observed by measurements showing the reduction in size of a tumor. In some embodiments, methods described herein result in the disappearance of target lesions (a tumor). This too may be observed by measurements showing the reduction in size and disappearance of a tumor.

In some embodiments, tumor evaluation comprises imaging the tumor to measure tumors and assess for any new tumors. A skilled clinician would know the best methods of imaging the tumor and patient for metastases. In some embodiments, imaging comprises computed tomography (CT) scan imaging. In some embodiments, imaging comprises magnetic resonance imaging (MRI). In some embodiments, imaging comprises positron emission tomography (PET) scan imaging. In some embodiments, imaging comprises ultrasound imaging. In some embodiments, imaging comprises CT scan imaging, MRI, PET scan imaging, or ultrasound imaging, or any combination thereof.

In some embodiments, tumor evaluation results in a decision to administer an IL-2 booster dose. In certain embodiments, an at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

In some embodiments, the administration of the one or more booster doses of IL-2 is prior to, concurrent with, or following the administration of said one or more additional doses of said anti-IL-2 antibody, one or more additional doses of said avelumab, or both.

In some embodiments, the anti-IL-2 antibody is administered at a dose of between about 0.5 mg/kg of a subject's body weight-12 mg/kg of said subject's body weight. In other embodiments, the anti-IL-2 antibody is administered at a dose of between about 4.5 mg/kg of a subject's body weight and 12 mg/kg of said subject's body weight. In other embodiments, the anti-IL-2 antibody is administered at a dose of between about 9 mg/kg of a subject's body weight and 12 mg/kg of said subject's body weight. In some embodiments, the anti-IL-2 antibody is administered at a dose of 9 mg/kg subject's body weight. In other embodiments, the anti-IL-2 antibody is administered at a dose of 12 mg/kg. In other embodiments, the anti-IL-2 antibody is administered at a dose of 6 mg/kg. In other embodiments, the anti-IL-2 antibody is administered at a dose of 3 mg/kg. In other embodiments, the anti-IL-2 antibody is administered at a dose of 10 mg/kg. In other embodiments, the anti-IL-2 antibody is administered at a dose of 15 mg/kg. In other embodiments, the anti-IL-2 antibody is administered at a dose of 20 mg/kg.

In some embodiments of a method of use of a combination therapy as described herein, the anti-IL-2 antibody is formulated for IV infusion.

In some embodiments, the methods described herein further comprise the step of administering one or more additional doses of the anti-IL-2 antibody. In other embodiments, the methods described herein further comprise the step of administering one or more additional doses of a pharmaceutical composition comprising the anti-IL-2 antibody.

In some embodiments, the anti-IL-2 antibody is administered on a bi-weekly (once every two weeks) schedule (FIG. 2). In other embodiments, the anti-IL-2 antibody is administered on a schedule as described herein above. In some embodiments, the anti-IL-2 antibody is administered weekly, bi-weekly (once every two weeks), once every three weeks, or once every four weeks.

In some embodiments, the schedule of administering the anti-IL-2 antibody may not be the same through the full course of the therapy, wherein administration may be bi-weekly for a time period and may then change to weekly, once every three weeks, once every four weeks, once every 5 weeks, or once every 6 weeks. Similarly, the schedule of administering the anti-IL-2 antibody may not be the same through the full course of the therapy, wherein administrations may be further apart, for example but not limited to once every 4-6 weeks and may then change to weekly, bi-weekly, or once every three weeks.

In some embodiments, the IL-2 is administered as a one-time loading dose and optionally as a booster dose as needed.

In some embodiments, the loading dose of IL-2 is administered at a dose of between about 15,000 IU/kg of said subject's body weight and 500,000 IU/kg of said subject's body weight. In other embodiments, the loading dose of IL-2 is administered at a dose of between about 45,000 and 270,000 IU/kg of the subject's body weight. In other embodiments, the loading dose of IL-2 is administered at a dose of between about 45,000 IU/kg and 135,000 IU/kg of said subject's body weight.

In some embodiments, the IL-2 loading dose is administered at a dose of 135,000 IU/kg of said subject's body weight. In other embodiments, the IL-2 loading dose is administered at a dose of 270,000 IU/kg. In other embodiments, the IL-2 loading dose is administered at a dose of 500,000 IU/kg. In other embodiments, the IL-2 loading dose is administered at a dose of 100,000 IU/kg. In other embodiments, the IL-2 loading dose is administered at a dose of 75,000 IU/kg. In other embodiments, the IL-2 loading dose is administered at a dose of 50,000 IU/kg.

In some embodiments, the booster dose of IL-2 is administered at a dose of between about 15,000 IU/kg of said subject's body weight and 500,000 IU/kg of said subject's body weight. In other embodiments, the booster dose of IL-2 is administered at a dose of between about 45,000 and 270,000 IU/kg of the subject's body weight. In other embodiments, the booster dose of IL-2 is administered at a dose of between about 45,000 IU/kg and 135,000 IU/kg of said subject's body weight.

In some embodiments, the IL-2 booster dose is administered at a dose of 135,000 IU/kg of said subject's body weight. In other embodiments, the IL-2 booster dose is administered at a dose of 270,000 IU/kg. In other embodiments, the IL-2 booster dose is administered at a dose of 500,000 IU/kg. In other embodiments, the IL-2 booster dose is administered at a dose of 100,000 IU/kg. In other embodiments, the IL-2 booster dose is administered at a dose of 75,000 IU/kg. In other embodiments, the IL-2 booster dose is administered at a dose of 50,000 IU/kg.

In some embodiments, the booster dose of IL-2 is administered once, twice, three times, four times, five times, or six times. In some embodiments, the booster dose of IL-2 is administered one week, two weeks, three weeks, four weeks, 5 weeks, 6 weeks, 7 weeks, or 8 weeks after the loading dose of IL-2. In some embodiments, the 2^{nd} booster dose of IL-2 is administered one week, two weeks, three weeks, four weeks, 5 weeks, 6 weeks, 7 weeks, or 8 weeks after the 1^{st} booster dose of IL-2. In some embodiments, a booster dose of IL-2 is administered one week, two weeks, three weeks, four weeks, 5 weeks, 6 weeks, 7 weeks, or 8 weeks after the prior dose of IL-2 to the subject.

In some embodiments, the anti-IL-2 antibody or composition thereof and the low dose IL-2 (loading or booster dose) or composition thereof are administered at a frequency or interval that is independent of one another.

In some embodiments, methods as described herein comprising the administration of an anti-IL-2 antibody or composition thereof and a low dose of IL-2 or composition thereof, the administration of the anti-IL-2 antibody or composition thereof and the administration of the low dose of IL-2 or composition thereof is concurrent. In other embodiments, the anti-IL-2 antibody or composition thereof is administered prior to the administration of the low dose of IL-2 or composition thereof. In other embodiments, the anti-IL-2 antibody or composition thereof is administered following the administration of the low dose of IL-2 or composition thereof.

In some embodiments, methods as described herein comprising the administration of an anti-IL-2 antibody or composition thereof and a loading dose of IL-2 or composition thereof, the administration of the anti-IL-2 antibody or composition thereof and the administration of the loading dose of IL-2 or composition thereof is concurrent. In other embodiments, the anti-IL-2 antibody or composition thereof is administered prior to the administration of the loading dose of IL-2 or composition thereof. In other embodiments, the anti-IL-2 antibody or composition thereof is administered following the administration of the loading dose of IL-2 or composition thereof.

In some embodiments, methods as described herein comprising the administration of an anti-IL-2 antibody or composition thereof and a booster dose of IL-2 or composition thereof, the administration of the anti-IL-2 antibody or composition thereof and the administration of the booster dose of IL-2 or composition thereof is concurrent. In other embodiments, the anti-IL-2 antibody or composition thereof is administered prior to the administration of the booster dose of IL-2 or composition thereof. In other embodiments, the anti-IL-2 antibody or composition thereof is administered following the administration of the booster dose of IL-2 or composition thereof.

In some embodiments, methods as described herein comprising the administration of an anti-IL-2 antibody or pharmaceutical composition thereof, a loading dose of IL-2 or pharmaceutical composition thereof, and an immune checkpoint inhibitor or a pharmaceutical composition thereof, wherein the administration of the anti-IL-2 antibody or pharmaceutical composition thereof, the administration of the loading dose of IL-2 or the pharmaceutical composition thereof, and or the administration of the immune checkpoint inhibitor or the pharmaceutical composition thereof is concurrent. In other embodiments, the anti-IL-2 antibody or composition thereof is administered prior to the administration of the loading dose of IL-2 or composition thereof and/or prior to the administration of the immune checkpoint inhibitor. In other embodiments, the anti-IL-2 antibody or composition thereof is administered following the administration of the loading dose of IL-2 or composition thereof and or the administration of the immune checkpoint inhibitor. In some embodiments, the immune checkpoint inhibitor is a PD-L1 immune checkpoint inhibitor. In some embodiments, the immune checkpoint inhibitor is a PD-1 immune checkpoint inhibitor. In some embodiments, the immune checkpoint inhibitor is avelumab.

In some embodiments, methods as described herein comprising the administration of an immune checkpoint inhibitor comprise the step of administering the immune checkpoint inhibitor or a pharmaceutical composition thereof prior to the administration of the anti-IL-2 antibody. In some embodiments, the immune checkpoint inhibitor is administered concurrent with the administration of the anti-IL-2 antibody. In some embodiments, the immune checkpoint inhibitor is administered following the administration of the anti-IL-2 antibody. In some embodiments, the immune checkpoint inhibitor is a PD-L1 immune checkpoint inhibitor. In some embodiments, the immune checkpoint inhibitor is a PD-1 immune checkpoint inhibitor. In some embodiments, the immune checkpoint inhibitor is avelumab.

In some embodiments, methods as described herein comprising the administration of an immune checkpoint inhibitor comprise the step of administering the immune checkpoint inhibitor or a pharmaceutical composition thereof prior to the administration of the low dose of IL-2. In some embodiments, the immune checkpoint inhibitor is administered concurrent with the administration of the low dose of IL-2. In some embodiments, the immune checkpoint inhibitor is administered following the administration of the low dose of IL-2. In some embodiments, the low dose of IL-2 is the loading dose of IL-2. In other embodiments, the low dose of IL-2 is the booster dose of IL-2. In some embodiments, the immune checkpoint inhibitor is a PD-L1 immune checkpoint inhibitor. In some embodiments, the immune checkpoint inhibitor is a PD-1 immune checkpoint inhibitor. In some embodiments, the immune checkpoint inhibitor is avelumab.

In some embodiments, methods as described herein comprising the administration of avelumab comprise the step of administering avelumab or a pharmaceutical composition thereof prior to the administration of the anti-IL-2 antibody. In some embodiments, avelumab is administered concurrent with the administration of the anti-IL-2 antibody. In some embodiments, avelumab is administered following the administration of the anti-IL-2 antibody.

In some embodiments, methods as described herein comprising the administration of avelumab comprise the step of administering avelumab or a pharmaceutical composition thereof prior to the administration of the low dose of IL-2. In some embodiments, avelumab is administered concurrent with the administration of the low dose of IL-2. In some embodiments, avelumab is administered following the administration of the low dose of IL-2.

In some embodiments, a method of use of a combination therapy as described herein comprises administering an immune checkpoint inhibitor by IV infusion. In some embodiments, a method of use of a combination therapy as described herein comprises administering an immune checkpoint inhibitor at a dose of 800 mg. In some embodiments, a method of use of a combination therapy as described herein comprises administering an immune checkpoint inhibitor at a dose of 800 mg by IV infusion. In some embodiments of a method of use of a combination therapy, the immune checkpoint inhibitor comprises avelumab.

In some embodiments, a method of use of a combination therapy as described herein comprises administering avelumab by IV infusion. In some embodiments, a method of use of a combination therapy comprises administering avelumab at a dose of 400 mg. In some embodiments, a method of use of a combination therapy comprises administering avelumab at a dose of 600 mg. In some embodiments, a method of use of a combination therapy comprises administering avelumab at a dose of 800 mg. In some embodiments, a method of use of a combination therapy comprises administering avelumab at a dose of 1000 mg. In some embodiments, a method of use of a combination therapy comprises administering avelumab at a dose of 1200 mg. In some embodiments, a method of use of a combination therapy comprises administering avelumab at a dose of 1400 mg. In some embodiments, a method of use of a combination therapy comprises administering avelumab at a dose of 1600 mg. In some embodiments, a method of use of a combination therapy comprises administering avelumab at a dose of 360-1600 mg. In some embodiments, a method of use of a combination therapy comprises administering avelumab at a dose of 600-1000 mg.

In some embodiments, a method of use of a combination therapy comprises administering avelumab at a dose of 400 mg by IV infusion. In some embodiments, a method of use of a combination therapy comprises administering avelumab at a dose of 600 mg by IV infusion. In some embodiments, a method of use of a combination therapy comprises administering avelumab at a dose of 800 mg by IV infusion. In some embodiments, a method of use of a combination therapy comprises administering avelumab at a dose of 1000 mg by IV infusion. In some embodiments, a method of use of a combination therapy comprises administering avelumab at a dose of 1200 mg by IV infusion. In some embodiments, a method of use of a combination therapy comprises administering avelumab at a dose of 1400 mg by IV infusion. In some embodiments, a method of use of a combination therapy comprises administering avelumab at a dose of 1600 mg by IV infusion. In some embodiments, a method of use of a combination therapy comprises administering avelumab at a dose of 360-1600 mg by IV infusion. In some embodiments, a method of use of a combination therapy comprises administering avelumab at a dose of 600-1000 mg by IV infusion.

In some embodiments, a method of use of a combination therapy comprises administering avelumab at a dose of 10 mg/kg of a subject's body weight. In some embodiment, a method of use of a combination therapy comprises administering avelumab at a dose of 10 mg/kg of a subject's body weight for subjects who weigh between 70-80 kg.

In some embodiments of a method of use treating an unresectable locally advanced or metastatic non-small cell lung cancer (NSCLC) that is PD-L1 positive, the method comprises administering 9 mg/kg AU-007 of a subject's body weight (administered intravenously (IV) once every two weeks (Q2W)), a loading dose of 135,000 IU/kg aldesleukin (IL-2) of a subject's body weight (administered subcutaneously (SC)), plus dose of avelumab 800 mg (IV) Q2W). In some embodiments of a method of use treating an unresectable locally advanced or metastatic non-small cell lung cancer (NSCLC) that is PD-L1 positive, the method comprises administering 9 mg/kg AU-007 of a subject's body weight (administered intravenously (IV) once every two weeks (Q2W)), a loading dose of 75,000 IU/kg aldesleukin (IL-2) of a subject's body weight (administered subcutaneously (SC)), plus dose of avelumab 800 mg (IV) Q2W). In some embodiments of a method of use treating an unresectable locally advanced or metastatic non-small cell lung cancer (NSCLC) that is PD-L1 positive, the method comprises administering 9 mg/kg AU-007 of a subject's body weight (administered intravenously (IV) once every two weeks (Q2W)), a loading dose of 45,000 IU/kg aldesleukin (IL-2) of a subject's body weight (administered subcutaneously (SC)), plus dose of avelumab 800 mg (IV) Q2W).

In some embodiments, methods as described herein comprising the administration of a PD-L1 immune checkpoint inhibitor comprise the step of administering the PD-L1 immune checkpoint inhibitor or a pharmaceutical composition thereof prior to the administration of the anti-IL-2 antibody. In some embodiments, the PD-L1 immune checkpoint inhibitor is administered concurrent with the administration of the anti-IL-2 antibody. In some embodiments, the PD-L1 immune checkpoint inhibitor is administered following the administration of the anti-IL-2 antibody.

In some embodiments, methods as described herein comprising the administration of the PD-L1 immune checkpoint inhibitor comprise the step of administering the PD-L1 immune checkpoint inhibitor or a pharmaceutical composition thereof prior to the administration of the low dose of IL-2. In some embodiments, the PD-L1 immune checkpoint inhibitor is administered concurrent with the administration of the low dose of IL-2. In some embodiments, the PD-L1 immune checkpoint inhibitor is administered following the administration of the low dose of IL-2.

In some embodiments, methods as described herein comprising the administration of a PD-1 immune checkpoint inhibitor comprise the step of administering the PD-1 immune checkpoint inhibitor or a pharmaceutical composition thereof prior to the administration of the anti-IL-2 antibody. In some embodiments, the PD-1 immune checkpoint inhibitor is administered concurrent with the administration of the anti-IL-2 antibody. In some embodiments, the PD-1 immune checkpoint inhibitor is administered following the administration of the anti-IL-2 antibody.

In some embodiments, methods as described herein comprising the administration of the PD-1 immune checkpoint inhibitor comprise the step of administering the PD-1 immune checkpoint inhibitor or a pharmaceutical composition thereof prior to the administration of the low dose of IL-2. In some embodiments, the PD-1 immune checkpoint inhibitor is administered concurrent with the administration of the low dose of IL-2. In some embodiments, the PD-1 immune checkpoint inhibitor is administered following the administration of the low dose of IL-2.

In some embodiments, the methods described herein further comprise the step of administering one or more additional doses of avelumab. In other embodiments, the methods described herein further comprise the step of administering one or more additional doses of a pharmaceutical composition comprising avelumab.

In some embodiments, the avelumab is administered at least once every week, bi-weekly (once every two weeks (Q2W)), once every three weeks (Q3W), once every four weeks (Q4W), once every five weeks (Q5W), or at least once every 6 weeks (Q6W). In some embodiments, the schedule of administering the avelumab may not be the same through the full course of the therapy, wherein administration may be bi-weekly for a time period and may then change to weekly, once every three weeks, once every four weeks, once every 5 weeks, or once every 6 weeks. Similarly, the schedule of administering the avelumab may not be the same through the full course of the therapy, wherein administrations may be further apart, for example but not limited to once every 4 to 6 weeks and may then change to weekly, bi-weekly (once every two weeks), or once every three weeks. In some embodiments of a method of use of a combination therapy disclosed herein, administration of avelumab to said subject is once every two weeks (FIG. 2).

In some embodiments of a method of use of a combination therapy described herein, the anti-IL-2 antibody is administered once every two weeks, a loading dose of IL-2 is administered, and the avelumab is administered once every two weeks. In certain embodiments, the method further comprises administering a IL-2 booster once every 8 weeks if analysis of one or more objective signs of worsening tumor growth kinetics indicates the booster is warranted. In some embodiments, one or more objective signs comprises previously shrinking tumors becoming stable, increase in tumor markers, tumor growth, or appearance of new tumor growth in a tumor that had previously been stable or had decreased in size, or any combination thereof. In some embodiments, with each cycle of treatment an analysis of one or more objective signs are performed, wherein a skilled clinician determines if an IL-2 booster dose should be administered as part of the method.

In certain embodiments, disclosed herein is a method of treating an unresectable locally advanced or metastatic non-small cell lung cancer (NSCLC) that is PD-L1 positive in a subject, said method comprising administering to said subject an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO: 2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5; wherein said NSCLC comprises unresectable locally advanced or metastatic NSCLC that is PD-L1 positive; wherein said treating comprises second-line or third-line treatment; wherein said pharmaceutical composition(s) further comprises a pharmaceutically acceptable carrier, thereby treating said NSCLC in said subject.

In certain embodiments, disclosed herein is a method of treating an unresectable locally advanced or metastatic non-small cell lung cancer (NSCLC) that is PD-L1 positive in a subject, said method comprising administering to said subject an anti-IL-2 antibody at a dose of 9 mg/kg of said subject's body weight or a pharmaceutical composition thereof, a loading dose of 75,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO: 2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5; wherein said NSCLC comprises unresectable locally advanced or metastatic NSCLC that is PD-L1 positive; wherein said treating comprises second-line or third-line treatment; wherein said pharmaceutical composition(s) further comprises a pharmaceutically acceptable carrier, thereby treating said NSCLC in said subject.

In certain embodiments, disclosed herein is a method of treating an unresectable locally advanced or metastatic non-small cell lung cancer (NSCLC) that is PD-L1 positive in a subject, said method comprising administering to said subject an anti-IL-2 antibody at a dose of 9 mg/kg of said subject's body weight or a pharmaceutical composition thereof, a loading dose of 45,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO: 2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5; wherein said NSCLC comprises unresectable locally advanced or metastatic NSCLC that is PD-L1 positive; wherein said treating comprises second-line or third-line treatment; wherein said pharmaceutical composition(s) further comprises a pharmaceutically acceptable carrier, thereby treating said NSCLC in said subject.

In certain embodiments, disclosed herein is a method of treating an unresectable locally advanced or metastatic non-small cell lung cancer (NSCLC) that is PD-1 positive in a subject, said method comprising administering to said subject an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO: 2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5; wherein said NSCLC comprises unresectable locally advanced or metastatic NSCLC that is PD-1 positive; wherein said treating comprises second-line or third-line treatment; wherein said pharmaceutical composition(s) further comprises a pharmaceutically acceptable carrier, thereby treating said NSCLC in said subject.

In some embodiments, the anti-IL-2 antibodies described and exemplified herein, bind the portion of IL-2 that interacts with the alpha (CD25) receptor subunit that is a component of the IL-2 trimeric receptor (CD25/CD132/CD122, sometimes represented as α/β/γ) found on Treg cells, eosinophils, and pulmonary and vascular endothelial cells. In some embodiments, the anti-IL-2 antibodies disclosed herein prevent activation of the trimeric IL-2 receptor found on Tregs, eosinophils, and pulmonary and vascular endothelial cells. In some embodiments, the anti-IL-2 antibodies disclosed herein bind to IL-2, activate signaling through the IL-2 dimer receptor (CD132/CD122, sometimes represented as β/γ) found on naïve Teff cells, NK cells, and Natural killer T (NKT) cells. Thus, in some embodiments, the present disclosure provides a method of preventing activation of the trimeric IL-2 receptor found on Tregs, eosinophils, and pulmonary and vascular endothelial cells. In other embodiments, the present disclosure provides a method of activating signaling through the IL-2 dimer receptor (CD132/CD122, sometimes represented as β/γ) found on naïve Teff cells, NK cells, and Natural killer T (NKT) cells.

In some embodiments, administration of an anti-IL-2 antibody disclosed herein, for example but not limited to AU-007, provides a method of preventing activation of the trimeric IL-2 receptor found on Tregs, eosinophils, and pulmonary and vascular endothelial cells and provides a method of activating signaling through the IL-2 dimer receptor (CD132/CD122, sometimes represented as β/γ) found on naïve Teff cells, NK cells, and Natural killer T (NKT) cells. A skilled artisan would appreciate that the anti-IL-2 antibody may function as an immunomodulator. Immunomodulators may either increase or decrease immune function. In some embodiments, an anti-IL-2 antibody described herein, for example AU-007, increases immune function. In other embodiments, an anti-IL-2 antibody described herein, for example AU-007, decreases immune functions. In some embodiments, an anti-IL-2 antibody, for example AU-007 may either increase or decrease immune function.

A complex of IL-2 and anti-IL-2 antibodies induced proliferation of memory phenotype effector T cells (MP) CD8+ cells and NK cells, while there was a much smaller effect on CD4+ Tregs. Thus, the engineered anti-IL-2 antibodies disclosed herein would be useful in adjusting immune cell populations and inducing differential expansion of certain immune effector cells. In some embodiments, such differential expansion of immune effect cells would result in robust activation of the immune system and could be useful for treatment of tumors. Thus, in some embodiments, the present disclosure provides a method of inducing proliferation of memory phenotype effector T cells (MP) CD8+ cells and NK cells. In other embodiments, the present disclosure provides a method of activating the immune system of a subject having an NSCLC.

In certain embodiments, the present disclosure provides a method of treating a cancer that progressed after prior checkpoint inhibitor therapy. In some embodiments, the present disclosure provides a method of treating a subject having cancer, wherein said subject was previously treated with an immune checkpoint inhibitor. In other embodiments, the subject being treated has a cancer that is recalcitrant to treatment with one or more immune checkpoint inhibitors.

In certain embodiments, the present disclosure provides a method of treating a cancer that progressed after prior PD-1 therapy. In some embodiments, the present disclosure provides a method of treating a subject having cancer, wherein said subject was previously treated with a PD-1 inhibitor. In other embodiments, the subject being treated has a cancer that is recalcitrant to treatment with one or more PD-1 inhibitors.

In certain embodiments, the present disclosure provides a method of treating a cancer that progressed after prior PD-L1 therapy. In some embodiments, the present disclosure provides a method of treating a subject having cancer, wherein said subject was previously treated with a PD-L1 inhibitor. In other embodiments, the subject being treated has a cancer that is recalcitrant to treatment with one or more PD-L1 inhibitors.

In some embodiments, a method as described herein further comprises administering to said subject an adjuvant treatment. In other embodiments, a method as described herein further comprises administering to said subject a neoadjuvant treatment. In other embodiments, a method as described herein further comprises administering to said subject an adjuvant treatment and a neoadjuvant treatment.

In some embodiments, an “adjuvant” treatment is administered after primary treatment, such as surgery, to eliminate residual cancer cells that may remain and reduce the risk of the cancer returning. In some embodiments, a “neoadjuvant” treatment is administered before the primary treatment, typically surgery, to shrink tumors, make surgery less invasive, treat micrometastases, and improve the overall success of cancer treatment. In some embodiments, the adjuvant or neo-adjuvant treatment comprises chemotherapy, radiation therapy, hormone therapy, targeted therapy, immunotherapy, or any combination thereof.

In some embodiments, the present disclosure provides a method for reducing the size of the NSCLC. In other embodiments, the present disclosure provides a method for inhibiting the growth of the NSCLC. In other embodiments, the present disclosure provides a method for reducing the growth of the NSCLC. In other embodiments, the present disclosure provides a method for inhibiting the metastases of the NSCLC. In other embodiments, the present disclosure provides a method for reducing the metastases of the NSCLC. In other embodiments, the present disclosure provides a method for increasing the time before detection of a new lesion. In other embodiments, the present disclosure provides a method for decreasing a target lesion. In other embodiments, the present disclosure provides a method for shrinking a target lesion. In other embodiments, the present disclosure provides a method for obliterating or eradicating a target lesion. In other embodiments, the present disclosure provides a method for removing a target lesion. In other embodiments, the present disclosure provides a method for causing a target lesion to disappear.

In some embodiments, treatment of an unresectable locally advanced or metastatic NSCLC in a subject reduces the size of the NSCLC, inhibits or reduces growth of the NSCLC, or inhibits or reduces metastases of said NSCLC, or any combination thereof.

In certain embodiments of a method of use of a combination therapy disclosed herein, the method comprises (i) reducing the size of the tumor, (ii) inhibiting or reducing growth of the tumor, (iii) inhibiting or reducing metastases of said tumor, (iv) inhibiting the production of new lesions, (v) decreasing or shrinking target lesions, (vi) eliminating target lesions, or (vii) any combination thereof.

In some embodiments, the method comprises the step of administering an anti-IL-2 antibody. In other embodiments, the method comprises the step of administering an anti-IL-2 antibody and a low loading dose of IL-2. In other embodiments, the method comprises administering an anti-IL-2 antibody and one or more immune checkpoint inhibitors. In other embodiments, the method comprises administering an anti-IL-2 antibody and avelumab. In other embodiments, the method comprises administering an anti-IL-2 antibody, a low loading dose of IL-2 and one or more immune checkpoint inhibitors. In other embodiments, the method comprises administering an anti-IL-2 antibody, a low loading dose of IL-2, and avelumab. In other embodiments, the method comprises administering an anti-IL-2 antibody, a low loading dose of IL-2, one or more low booster doses of IL-2, and one or more immune checkpoint inhibitors. In other embodiments, the method comprises administering an anti-IL-2 antibody, a low loading dose of IL-2, one or more low booster doses of IL-2, and avelumab.

As used throughout, the terms “cancer” and “tumor” may in some embodiments be used interchangeably having the same meanings and qualities.

In other embodiments, the present disclosure provides a method of treating an unresectable NSCLC. In other embodiments, the present disclosure provides a method of treating a locally advanced NSCLC. In other embodiments, the present disclosure provides a method of treating an unresectable locally advanced NSCLC. In some embodiments, the present disclosure provides a method of treating a metastatic NSCLC.

In other embodiments, a subject treated by a method disclosed herein has an unresectable NSCLC. In other embodiments, a subject treated by a method disclosed herein has a locally advanced NSCLC. In other embodiments, a subject treated by a method disclosed herein has an unresectable locally advanced NSCLC. In some embodiments, a subject treated by a method disclosed herein has a metastatic NSCLC.

In some embodiments, an unresectable tumor is a tumor that is not considered a candidate for surgical removal due to factors like advanced stage, metastasis, or being too large, too close to vital structures, or too difficult to access. In some embodiments, an unresectable tumor is an inoperable tumor having a technical or anatomical barrier to resection, such as complete encasement of a major blood vessel, that makes complete surgical removal uncertain or impossible.

In some embodiments, the NSCLC comprises an immune sensitive NSCLC.

In some embodiments, treatment of an unresectable locally advanced or metastatic NSCLC in a subject as described herein comprises maintenance treatments. In some embodiments, maintenance treatments are administered to maintain the absence of an unresectable locally advanced or metastatic NSCLC. In some embodiments, maintenance treatments are administered to maintain lack of metastasis of an unresectable locally advanced or metastatic NSCLC. In some embodiments, maintenance treatments are administered to inhibit metastasis of an unresectable locally advanced or metastatic NSCLC. In some embodiments, maintenance treatments are administered to maintain lack of growth of an unresectable locally advanced or metastatic NSCLC. In some embodiments, maintenance treatments are administered to inhibit growth of an unresectable locally advanced or metastatic NSCLC.

In some embodiments, treatment of NSCLC comprises prophylactic treatment of, for example, but not limited to, a subject harboring a genetic marker or markers with a high risk of developing NSCLC.

In some embodiments, the present disclosure provides a method of promoting differential growth of immune cells in a subject, comprising the step of preparing a composition comprising an anti-IL-2 antibody disclosed herein, and administering the composition to the subject, thereby promoting differential growth of immune cells in the subject. In some embodiments, the present disclosure provides a method of promoting differential growth of immune cells in a subject, comprising the step of preparing a composition comprising IL-2 and the anti-IL-2 antibody disclosed herein, and administering the composition to the subject, thereby promoting differential growth of immune cells in the subject. In some embodiments, the subject can be an animal or a human. In some embodiments, the immune cells can be CD8⁺ cells or NK cells.

In some embodiments, disclosed herein is a method of treating an unresectable locally advanced or metastatic NSCLC in a subject, comprising the step of administering to the subject a composition comprising an anti-IL-2 antibody as disclosed herein, wherein said antibody promotes differential growth of subsets of immune cells and decreases undesirable effects caused by IL-2, thereby treating unresectable locally advanced or metastatic NSCLC in said subject. In some embodiments, a method of treating a disease disclosed here comprises use of a composition comprising an anti-IL-2 antibody and IL-2. In some embodiments, a method of treating a disease comprises treating an unresectable locally advanced or metastatic NSCLC. In some embodiments, a method of treating a condition comprises treating a weak immune system and the treatment prophylactically boosts the immune system.

In some embodiments, methods of promoting differential growth of immune cells in a subject, comprise the step of preparing and administering a composition comprising an anti-IL-2 antibody disclosed herein. In some embodiments, methods of promoting differential growth of immune cells in a subject, comprise the step of preparing and administering a composition comprising IL-2. In some embodiments, methods of promoting differential growth of immune cells in a subject, comprise the step of preparing and administering a composition comprising an immune checkpoint inhibitor. In some embodiments, methods of promoting differential growth of immune cells in a subject, comprise the step of preparing and administering a composition comprising avelumab. In some embodiments, methods of promoting differential growth of immune cells in a subject, comprise the step of preparing and administering an anti-IL-2 antibody and IL-2 or composition(s) thereof. In some embodiments, the IL-2 is a loading dose of IL-2. In some embodiments, the IL-2 is a booster dose of IL-2. In some embodiments, the loading dose or booster dose or both of IL-2 is low dose IL-2.

In some embodiments, methods of promoting differential growth of immune cells in a subject, comprise the step of preparing and administering an anti-IL-2 antibody and an immune checkpoint inhibitor or composition(s) thereof. In other embodiments, methods of promoting differential growth of immune cells in a subject, comprise the step of preparing and administering an anti-IL-2 antibody and a PD-L1 inhibitor or a PD-1 inhibitor or composition(s) thereof. In other embodiments, methods of promoting differential growth of immune cells in a subject, comprise the step of preparing and administering an anti-IL-2 antibody and avelumab or composition(s) thereof. In other embodiments, the method comprises preparing and administering an anti-IL-2 antibody, an immune checkpoint inhibitor, and IL-2 or composition(s) thereof. In other embodiments, the method comprises preparing and administering an anti-IL-2 antibody, a PD-L1 inhibitor or a PD-1 inhibitor, and IL-2 or composition(s) thereof. In other embodiments, the method comprises preparing and administering an anti-IL-2 antibody, avelumab, and IL-2 or composition(s) thereof. In some embodiments, the IL-2 is a loading dose of IL-2. In some embodiments, the IL-2 is a booster dose of IL-2. In some embodiments, the loading dose or booster dose or both of IL-2 is low dose IL-2.

In some embodiments, the methods described herein comprise the steps of:
(a) preparing a composition comprising an anti-IL-2 antibody as disclosed herein; and

(b) administering the composition from (a) to the subject.

In some embodiments, the methods described herein comprise the steps of:
(a) preparing a composition comprising an anti-IL-2 antibody as disclosed herein;
(b) preparing a composition comprising IL-2 as disclosed herein;
(c) preparing a composition comprising an immune checkpoint inhibitor as disclosed herein; and
(d) administering the composition from (a), (b), and (c) as disclosed herein, to the subject. In some embodiments, the immune checkpoint inhibitor comprises a PD-L1 checkpoint inhibitor. In other embodiments, the immune checkpoint inhibitor comprises a PD-1 checkpoint inhibitor. In some embodiments, the immune checkpoint inhibitor is avelumab.

In some embodiments, the methods described herein comprise the steps of:
(a) preparing a composition comprising an anti-IL-2 antibody as disclosed herein;
(b) preparing a composition comprising a loading dose of IL-2 as disclosed herein;
(c) preparing a composition comprising an immune checkpoint inhibitor as disclosed herein; and
(d) administering the composition from (a), (b), and (c) as disclosed herein, to the subject, wherein additional doses of the anti-IL-2 antibody are administered for the duration of the treatment, and additional doses of the immune checkpoint inhibitor are administered for the duration of the treatment, and if analysis of objective signs warrants, an at least one booster IL-2 dose is administered. In some embodiments, the immune checkpoint inhibitor comprises a PD-L1 checkpoint inhibitor. In other embodiments, the immune checkpoint inhibitor comprises a PD-1 checkpoint inhibitor. In some embodiments, the immune checkpoint inhibitor is avelumab.

In some embodiments, the methods described herein comprise the steps of:
(a) preparing a composition comprising an anti-IL-2 antibody as disclosed herein;
(b) preparing a composition comprising a loading dose of IL-2 as disclosed herein;
(c) preparing a composition comprising avelumab as disclosed herein; and
(d) administering the composition from (a), (b), and (c) as disclosed herein, to the subject, wherein additional doses of the anti-IL-2 antibody are administered for the duration of the treatment, and additional doses of avelumab are administered for the duration of the treatment, and if analysis of objective signs warrants, an at least one booster IL-2 dose is administered.

In some embodiments, the subject can be an animal or a human.

In some embodiments, treatment with the anti-IL-2 antibodies expands immune cells comprising one or more of naïve T cells, memory T cells, CD8⁺ T cells, NK cells, and Natural Killer T cells. In some embodiments, treatment with the anti-IL-2 antibodies decreases one or more undesirable effects caused by IL-2 such as activation of regulatory T cells, apoptosis of CD25⁺ T effector cells, pulmonary edema, pneumonia, and IL-2-induced vascular leakage.

In some embodiments, an anti-IL-2 antibody as described herein is engineered or modified. In some embodiments, the engineered or modified anti-IL-2 antibodies comprise a heavy chain variable region and a light chain variable region having the sequences of SEQ ID NO: 6 and SEQ ID NO:7.

In another embodiment, the engineered or modified anti-IL-2 antibodies comprise a heavy chain variable region having complementarity determining region (CDR) 1, CDR2 and CDR3. In some embodiments, the heavy chain CDR1, CDR2 and CDR3 comprise amino acid sequences of SEQ ID NOs: 1-3, respectively.

In other embodiments, the engineered or modified anti-IL-2 antibodies comprise a light chain variable region having complementarity determining region (CDR) 1, CDR2 and CDR3. In some embodiments, the light chain CDR1, CDR2 and CDR3 comprise amino acid sequences of SEQ ID NO:4, DAS, and SEQ ID NO: 5 respectively.

In other embodiments, the amino acid sequence of the full length heavy chain of the anti-IL-2 antibody is as set forth in SEQ ID NO: 8 and the amino acid sequence of the full length light chain of the anti-IL-2 antibody is as set forth in SEQ ID NO: 9. In some embodiments, the engineered anti-IL-2 antibody is an IgG. In other embodiments, the engineered anti-IL-2 antibody is an IgA. In other embodiments, the engineered anti-IL-2 antibody is an IgM.

In other embodiments, the engineered anti-IL-2 antibody is an IgE. In other embodiments, the engineered anti-IL-2 antibody is an IgD. In other embodiments, the engineered anti-IL-2 antibody is a Fv. In other embodiments, the engineered anti-IL-2 antibody is a scFv. In other embodiments, the engineered anti-IL-2 antibody is a Fab. In other embodiments, the engineered anti-IL-2 antibody is a F(ab′)₂. In some embodiments, the IgG is of the subclass of IgG1. In other embodiments, the IgG is of the subclass of IgG2. In other embodiments, the IgG is of the subclass of IgG3. In other embodiments, the IgG is of the subclass of IgG4. In some embodiments, the engineered antibody is part of a minibody. In other embodiments, the engineered antibody is part of a diabody. In other embodiments, the engineered antibody is part of a triabody antibody.

In some embodiments, the engineered anti-IL-2 antibody comprises a heavy chain comprising a mutation that reduces binding to a fragment crystallizable gamma receptor (FcγR; Fcγ receptor). In some embodiments, the mutation comprises L234A, L235A mutations.

In some embodiments, the polypeptides disclosed herein may be administered to a subject directly, or by administering to the subject a nucleic acid sequence encoding the polypeptides. In some embodiments, the nucleic acid sequence may be carried by a vector.

In some embodiments, a polynucleotide sequence encoding an engineered anti-IL-2 antibody is used in a method of treating a subject with a disease or condition as described herein, wherein the polynucleotide encodes an antibody comprising a heavy chain variable region having the amino acid sequence of SEQ ID NO:6 In some embodiments, a polynucleotide sequence encoding an engineered anti-IL-2 antibody is used in a method of treating a subject with a disease or condition as described herein, wherein the polynucleotide encodes an antibody comprising a light chain variable region having the amino acid sequence of SEQ ID NO:7. In some embodiments, a polynucleotide sequence encoding an engineered anti-IL-2 antibody is used in a method of treating a subject with a disease or condition as described herein, wherein the polynucleotide encodes an antibody comprising a heavy chain variable region and a light chain variable region having the amino acid sequences of one of SEQ ID NOs: 6 and 7.

In some embodiments of a method of using a polynucleotide to treat a disease or condition as described above, the polynucleotide encodes an engineered anti-IL-2 antibody that can be an IgG, IgA, IgM, IgE, IgD, a Fv, a scFv, a Fab, or a F(ab′)₂. The IgG can be of the subclass of IgG1, IgG2, IgG3, or IgG4. In some embodiments, the polynucleotide encodes an engineered antibody which is part of a minibody, a diabody, or a triabody antibody.

In some embodiments a polynucleotide sequence encoding an engineered anti-IL-2 antibody is used in a method of treating a subject with a disease or condition as described herein, wherein the polynucleotide sequence comprises the sequence of SEQ ID NO: 10:

(caggtccaactggtgcagtccggtgccgaagttaaaaaacctgggtct
tccgttaaagtttcttgcaaagcctctggctacagcatcaccgatgacc
tgattcactgggtccgtcaggctccaggtcaaggtctggaatggatggg
ttggatcgatccagaagacggtgaaaccaactatgcccagaaattccag
ggtcgtgtaaccctgaccgccgacacctccacctctaccgcctacatgg
agttaagtagcctgcgttcagaggataccgcagtgtactactgcgctcg
ttcactggactccacctggatctacccattcgcatactggggtcagggc
accctggtaaccgttagtagcggcggtggtggtagcggaggcggaggat
caggtggaggcggcagtgacatcgtgatgacccagtctcctgactcctt
ggccgtctctctgggcgaacgtgcaactatcaactgcaaatccagccag
agcttactgcgtcgcggtaatcagaaaaaccaccttgcatggtatcagc
agaaaccaggtcagccaccaaaattactgatctatgacgcatctaccgg
tcaaagcggtgtcccagatcgtttcagcggttccggctccggtactgac
ttcaccctgaccatctcttcccttcaggccgaagatgtggccgtgtatt
actgcctgcagagctacatcaccccacctactttcggtgctggtactaa
agttgaaatcaaa; SEQ ID NO: 10).

In some embodiments of a method of treating a disease or condition as described herein, the immune effector cells that are activated by the treatment are CD8+ cells or NK cells. In some embodiments, the anti-IL-2 antibodies disclosed herein, or a complex of IL-2 and the anti-IL-2 antibodies disclosed herein, exhibits pronounced effect in inducing proliferation of MP CD8⁺ cells and NK cells, while there was much smaller effect on CD4+ Tregs. In certain embodiments, there is no effect on CD4⁺ Tregs.

In certain embodiments, methods of use of an anti-IL-2 antibody disclosed herein provide a pro-stimulatory effect. In some embodiments, said use comprises the anti-IL-2 antibody. In some embodiments, said use comprises the anti-IL-2 antibody and an IL-2 (loading or booster dose), a PD-1 inhibitor, a PD-L1 inhibitor, avelumab, or a combination thereof.

Thus, the engineered anti-IL-2 antibodies disclosed herein are useful in adjusting immune cell populations and inducing differential expansion of certain immune effector cells in a method of treating a disease such as an unresectable locally advanced or metastatic NSCLC, or treating a condition such as IL-2 induced pulmonary edema, or IL-2-induced vascular leakage.

In some embodiments, a method disclosed herein comprising administering an anti-IL-2 antibody or a pharmaceutical composition thereof, a loading dose of IL-2 or a pharmaceutical composition thereof, and avelumab or a pharmaceutical composition thereof. In some embodiments, a subject is treated with said avelumab concurrently, before, or after treatment with an anti-IL-2 antibody. In some embodiments, a subject is treated with said avelumab concurrently, before, or after treatment with low dose IL-2, which in some embodiments, is a loading does and in other embodiments, is a booster dose.

In some embodiments of a method of use of a combination therapy as described herein, a subject comprises a mammalian subject. In some embodiments, a subject comprises a human subject. In some embodiments, a subject suffers from immune deficiency. Treatment of an immune deficient subject would, in some embodiments, comprise a prophylactic treatment.

### Combination Therapies

In some embodiments, an anti-IL-2 antibody or composition thereof as disclosed herein, is used as part of a combination therapy. In some embodiments, an anti-IL-2 antibody or composition thereof as disclosed herein, is used in combination with an immune checkpoint inhibitor. In some embodiments, an anti-IL-2 antibody or composition thereof as disclosed herein, is used in combination with IL-2. In some embodiments, an anti-IL-2 antibody or composition thereof as disclosed herein, is used in combination with IL-2 and with an immune checkpoint inhibitor. In some embodiments, the IL-2 is low-dose IL-2. In some embodiments, the IL-2 is a loading dose of IL-2. In some embodiments, the IL-2 is a booster dose of IL-2. In some embodiments, IL-2 is administered as a loading dose and as a booster dose at a later time period depending on the need of the patient.

In some embodiments, an anti-IL-2 antibody or a composition thereof, is used in combination with an immune checkpoint inhibitor. In some embodiments, the term “immune checkpoint inhibitor” may encompass any compound or molecule capable of inhibiting the function of a checkpoint protein. In some embodiments, the term “immune checkpoint inhibitor” may encompass any compound or molecule which targets immune checkpoint proteins. An artisan would appreciate that “immune checkpoints” are key regulators of the immune system that when stimulated can dampen the immune response to an immunologic stimulus. Checkpoint inhibitors can block inhibitory checkpoints, and thereby restore immune system function. In some embodiments, the one or more checkpoint inhibitors comprise immune checkpoint inhibitors.

A skilled artisan would appreciate that the terms “immune checkpoint inhibitors” (ICIs), “checkpoint inhibitors,” and the like may be used interchangeably herein having all the same qualities and meanings, wherein an immune checkpoint inhibitor encompasses compounds that inhibit the activity or control mechanism(s) of the immune system. Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by inhibiting the activity of a protein in the pathway.

Immune checkpoint inhibitor targets include, but are not limited to PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, and VTCN-1. In some embodiments, an anti-IL-2 antibody therapy is used in combination with an immune checkpoint inhibitor, wherein the target of the immune checkpoint inhibitor comprises PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, or any combination thereof.

Checkpoint inhibitors may include antibodies, or antigen binding fragments thereof, other binding proteins, biologic therapeutics, or small molecules, that bind to and block or inhibit the activity of one or more of PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1. Illustrative checkpoint inhibitors include but are not limited to those listed in Table 1 below.

TABLE 1
Non-Limiting Examples of Checkpoint Inhibitors
and the Immune Checkpoint Inhibitor Target.

| Drug name | ICI Target |
|---|---|
| Avelumab (Bavencio ®) | anti-PD-L1 |
| Atezolizumab (Tecentriq ®) | anti-PD-L1 |
| Durvalumab (Imfinzi ®) | anti-PD-L1 |
| Sugemalimab (Cejemly ®) | anti-PD-L1 |
| Envafolimab | anti-PD-L1 |
| Cosibelimab (Unloxcyt ®) | anti-PD-L1 |
| Nivolumab (Opdivo ®)-BMS | anti-PD-1 |
| Pembrolizumab(Keytruda ®) | anti-PD-1 |
| Cemiplimab (Libtayo ®) | anti-PD-1 |
| Camrelizumab | anti-PD-1 |
| Zimberelimab (Glimta ®) | anti-PD-1 |
| Tislelizumab (Tevimbra ®) | anti-PD-1 |
| Sintilimab (Tyvyt ®) | anti-PD-1 |
| Teriprizumab | anti-PD-1 |
| Prolgolimab | anti-PD-1 |
| Penpulimab | anti-PD-1 |
| Dostarlimab (Jemperli ®) | anti-PD-1 |
| Genolimzumab | anti-PD-1 |
| Retifanlimab (Zynyz ®) | anti-PD-1 |
| Ipilimumab (Yervoy ®) | anti-CTLA4 |
| Tiragolumab | anti-TIGIT |
| Domvanalimab | anti-TIGIT |
| Vibostolimab | anti-TIGIT |
| BMS-986207 | anti-TIGIT |
| EOS-448 (belrestotug ®) | anti-TIGIT |
| COM-902 | anti-TIGIT |
| Sabatolimab | anti-TIM3 |
| Cobolimab | anti-TIM3 |
| BMS-986258 | anti-TIM3 |
| INCAGN-02390 | anti-TIM3 |
| S-95018 | anti-TIM3 |
| Omburtamab | anti-B7-H3 |
| MGC-018 | anti-B7-H3 |
| Enoblituzumab | anti-B7-H3 |
| Oleclumab | anti-CD73 |
| BMS-986179 | anti-CD73 |
| NZV-930 | anti-CD73 |
| CPX-006 | anti-CD73 |
| MK-4280 (favezelimab ®) | anti-LAG3 |
| Relatlimab | anti-LAG3 |
| Relatlimab + Nivolumab | anti-LAG3 + anti-PD-1 |
| (Opdualag ™) | |
| Sym-022 | anti-LAG3 |
| Ieramilimab | anti-LAG3 |
| BI-754111 (miptenalimab ®) | anti-LAG3 |
| MK-5890 (boserolimab ®) | anti-CD27 |
| Varlilumab | anti-CD27 |
| Cusatuzumab | anti-CD70 |
| Vorsetuzumab | anti-CD70 |
| Urelumab | anti-4-1BB (agonist) |
| Utomilumab | anti-4-1BB (agonist) |
| ATOR-1017 (evunzekibart ®) | anti-4-1BB (agonist) |
| RO-7122290 | anti-4-1BB (agonist) |
| INCAGN-01876 (ragifilimab ®) | anti-GITR (agonist) |
| BMS-986156 | anti-GITR (agonist) |
| TRX-518 | anti-GITR (agonist) |
| GWN-323 | anti-GITR (agonist) |
| BMS-986178 | anti-OX40 (agonist) |
| INCAGN-1949 | anti-OX40 (agonist) |
| GSK-3174998 | anti-OX40 (agonist) |
| BGB-A-445 | anti-OX40 (agonist) |
| BI-765063 | anti- SIRP-alpha (CD47) |
| ALX-148 (evorpacept ®) | SIRP-alpha (CD47) |
| IPH-52 | anti-CD39 |
| TTX-030 | anti-CD39 |
| BAY-1905254 (bapotylimab ®) | anti-ILDR2 |
| Onvatilimab | anti-VISTA |
| K01401-020 | anti-VISTA |
| JS-004 | anti-BTLA |
| FPA-150 | anti-VTCN1 |

In some embodiments, the checkpoint inhibitor comprises a PD-1 inhibitor. In some embodiments, the checkpoint inhibitor comprises a PD-L1 inhibitor. In some embodiments, the checkpoint inhibitor comprises a CTLA-4 inhibitor. In some embodiments, the checkpoint inhibitor comprises a TIGIT inhibitor. In some embodiments, the checkpoint inhibitor comprises a TIM-3 inhibitor. In some embodiments, the checkpoint inhibitor comprises a B7-H3 inhibitor. In some embodiments, the checkpoint inhibitor comprises a CD73 inhibitor. In some embodiments, the checkpoint inhibitor comprises a LAG3 inhibitor. In some embodiments, the checkpoint inhibitor comprises a CD27 inhibitor. In some embodiments, the checkpoint inhibitor comprises a CD70 inhibitor. In some embodiments, the checkpoint inhibitor comprises a 4-1BB agonist binder. In some embodiments, the checkpoint inhibitor comprises a GITR agonist binder. In some embodiments, the checkpoint inhibitor comprises a OX40 agonist binder. In some embodiments, the checkpoint inhibitor comprises a SIRP-alpha (CD47) inhibitor. In some embodiments, the checkpoint inhibitor comprises a CD39 inhibitor. In some embodiments, the checkpoint inhibitor comprises a ILDR2 inhibitor. In some embodiments, the checkpoint inhibitor comprises a VISTA inhibitor. In some embodiments, the checkpoint inhibitor comprises a BTLA inhibitor. In some embodiments, the checkpoint inhibitor comprises a VTCN-1 inhibitor.

In some embodiments, a pharmaceutical composition for use in a combination therapy, as described herein, comprises an effective amount of a checkpoint inhibitor, as described herein, and a pharmaceutically acceptable carrier.

In some embodiments, a composition disclosed herein comprises a checkpoint inhibitor and a pharmaceutically acceptable carrier. In some embodiments, a composition comprises a checkpoint inhibitor comprising a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor.

In some embodiments, a combination therapy comprises use of an anti-IL-2 antibody or composition thereof as described herein, and a checkpoint inhibitor or a composition thereof. In some embodiments, a combination therapy comprises use of an anti-IL-2 antibody or composition thereof and IL-2 as described herein, and a checkpoint inhibitor or a composition thereof.

As used herein, in some embodiments the terms “combination” and “combination therapy” may be used interchangeably having all the same meanings and qualities. In other embodiments, a combination of the present disclosure comprises an anti-IL-2 antibody or composition thereof as described herein, and a checkpoint inhibitor or a composition thereof. In some embodiments, a combination of the present disclosure comprises an anti-IL-2 antibody or composition thereof and IL-2 or a composition thereof as described herein, and a checkpoint inhibitor or a composition thereof.

In some embodiments, a combination of the present disclosure comprises an anti-IL-2 antibody or composition thereof and low dose of IL-2 or composition thereof as described herein, and a checkpoint inhibitor or a composition thereof. In some embodiments, a combination of the present disclosure comprises an anti-IL-2 antibody or composition thereof and low dose loading dose (low loading dose) of IL-2 or composition thereof as described herein, and a checkpoint inhibitor or a composition thereof. In some embodiments, a combination of the present disclosure comprises an anti-IL-2 antibody or composition thereof and a low dose booster dose (low booster dose) of IL-2 or composition thereof as described herein, and a checkpoint inhibitor or a composition thereof.

In some embodiments, a combination therapy as described herein comprises an anti-IL-2 antibody formulated for IV infusion and administration at a dose of 9 mg/kg. In other embodiments, the anti-IL-2 antibody is formulated for IV infusion and administration at a dose of between about 0.5 mg/kg of a subject's body weight and 12 mg/kg of said subject's body weight. In other embodiments, the anti-IL-2 antibody is formulated for IV infusion and administration at a dose of between about 4.5 mg/kg of a subject's body weight and 12 mg/kg of said subject's body weight. In other embodiments, the anti-IL-2 antibody is formulated for IV infusion and administration at a dose of between about 9 mg/kg of a subject's body weight and 12 mg/kg of said subject's body weight. In some embodiments, a combination therapy as described herein comprises an anti-IL-2 antibody formulated for intravenous (IV) administration.

In some embodiments, a combination therapy as described herein comprises an anti-IL-2 antibody formulated for administration at a dose of 9 mg/kg. In other embodiments, the anti-IL-2 antibody is formulated administration at a dose of between about 0.5 mg/kg of a subject's body weight and 12 mg/kg of said subject's body weight. In other embodiments, the anti-IL-2 antibody is formulated for administration at a dose of between about 4.5 mg/kg of a subject's body weight and 12 mg/kg of said subject's body weight. In other embodiments, the anti-IL-2 antibody is formulated for administration at a dose of between about 9 mg/kg of a subject's body weight and 12 mg/kg of said subject's body weight. In some embodiments, a combination therapy as described herein comprises an anti-IL-2 antibody formulated for intravenous (IV) administration.

In some embodiments, a combination therapy as described herein comprises a loading low dose of IL-2 at a dose of 135,000 IU/kg. In some embodiments, a combination therapy as described herein comprises a booster dose of IL-2 at a dose of 135,000 IU/kg. In some embodiments, the loading or booster IL-2 dose is formulated for subcutaneous injection. In some embodiments, the loading or booster dose is formulated for subcutaneous injection and administration at a dose of between about 45,000 IU/kg of a subject's body weight and 270,000 IU/kg of said subject's body weight. In other embodiments, the loading or booster dose is formulated for subcutaneous injection and administration at a dose of between about 45,000 IU/kg of a subject's body weight and 135,000 IU/kg of said subject's body weight. In other embodiments, the loading or booster dose is formulated for subcutaneous injection and administration at a dose of between 15,000 IU/kg of said subject's body weight 500,000 IU/kg of said subject's body weight. In some embodiments, a combination therapy as described herein comprises a low dose of IL-2 formulated for subcutaneous injection and administration at 135,000 IU/kg of said subject's body weight.

In some embodiments, a combination therapy as described herein comprises the immune checkpoint inhibitor formulated for IV infusion and administration at a dose of 800 mg. In some embodiments, a combination therapy as described herein comprises avelumab formulated, e.g., for IV infusion, and administration at a dose of 800 mg. In some embodiments, a combination therapy as described herein comprises an immune checkpoint inhibitor formulated, e.g., for IV infusion, and administration. In some embodiments, a combination therapy as described herein comprises avelumab formulated, e.g., for IV infusion and administration. In some embodiments, the immune checkpoint inhibitor comprises avelumab.

In some embodiments, a combination therapy as described herein comprises avelumab formulated for IV infusion and administration at a dose of 400 mg. In some embodiments, a combination therapy as described herein comprises avelumab formulated for IV infusion and administration at a dose of 600 mg. In some embodiments, a combination therapy as described herein comprises avelumab formulated for IV infusion and administration at a dose of 800 mg. In some embodiments, a combination therapy as described herein comprises avelumab formulated for IV infusion and administration at a dose of 1000 mg. In some embodiments, a combination therapy as described herein comprises avelumab formulated for IV infusion and administration at a dose of 1200 mg. In some embodiments, a combination therapy as described herein comprises avelumab formulated for IV infusion and administration at a dose of 1400 mg. In some embodiments, a combination therapy as described herein comprises avelumab formulated for IV infusion and administration at a dose of 1600 mg. In some embodiments, a combination therapy as described herein comprises avelumab formulated for IV infusion and administration at a dose of 360-1600 mg. In some embodiments, a combination therapy as described herein comprises avelumab formulated for IV infusion and administration at a dose of 600-1000 mg.

In some embodiments, disclosed herein is a combination therapy comprising 9 mg/kg AU-007 of a subject's body weight (formulated for intravenous (IV) administration), a loading dose of 135,000 IU/kg aldesleukin (IL-2) of a subject's body weight (formulated for subcutaneous (SC) administration), and 800 mg avelumab (formulated for IV administration). In some embodiments, a combination therapy comprises 9 mg/kg AU-007 of a subject's body weight (formulated for intravenous (IV) administration), a loading dose of 75,000 IU/kg aldesleukin (IL-2) of a subject's body weight (formulated for subcutaneous (SC) administration), and 800 mg avelumab (formulated for IV administration). In some embodiments, a combination therapy comprises 9 mg/kg AU-007 of a subject's body weight (formulated for intravenous (IV) administration), a loading dose of 45,000 IU/kg aldesleukin (IL-2) of a subject's body weight (formulated for subcutaneous (SC) administration), and 800 mg avelumab (formulated for IV administration).

In certain embodiments, disclosed herein is a combination therapy comprising an anti-IL-2 antibody or a pharmaceutical composition thereof, a loading dose of IL-2 or a pharmaceutical composition thereof, and avelumab or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:1, said HCDR2 comprises the amino acid sequence of SEQ ID NO: 2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5; wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said avelumab is formulated for administration at a dose of 800 mg; and wherein said pharmaceutical composition(s) further comprises a pharmaceutically acceptable carrier. In some embodiments, the combination therapy may be used for treating unresectable locally advanced or metastatic solid cancer. In some embodiments, the combination therapy may be used for treating unresectable locally advanced or metastatic non-small cell lung cancer (NSCLC). In some embodiments, the combination therapy may be used for treating unresectable locally advanced or metastatic non-small cell lung cancer (NSCLC) that is PD-L1 positive. In some embodiments, the combination therapy may be used for treating unresectable locally advanced or metastatic non-small cell lung cancer (NSCLC) that is PD-L1 positive, wherein treatment consists of 2nd line or 3rd line treatment. In other embodiments, the combination therapy may be used for treating unresectable locally advanced or metastatic non-small cell lung cancer (NSCLC) that is PD-1 positive. In other embodiments, the combination therapy may be used for treating unresectable locally advanced or metastatic non-small cell lung cancer (NSCLC) that is PD-1 positive, wherein treatment consists of 2nd line or 3rd line treatment.

In some embodiments, a combination therapy may include an immune checkpoint inhibitor targeting PD-1 or PD-L1. In some embodiments the immune checkpoint inhibitor targeting PD-1 comprises any of nivolumab, pembrolizumab, cemiplimab, camrelizumab, zimberelimab, tislelizumab, sintilimab, teriprizumab, prolgolimab, penpulimab, dostarlimab, genolimzumab, or retifanlimab. In some embodiments the immune checkpoint inhibitor targeting PD-L1 comprises any of avelumab, atezolizumab, durvalumab, sugemalimab, envafolimab, or cosibelimab.

In some embodiments, a combination therapy as described herein comprises avelumab formulated for IV infusion and administration at a dose of 800 mg. In some embodiments, a combination therapy as described herein comprises avelumab formulated for IV administration. In some embodiments, the immune checkpoint inhibitor comprises avelumab.

In some embodiments, a combination therapy as described herein comprises avelumab formulated for IV infusion and administration at a dose of 400 mg. In some embodiments, a combination therapy as described herein comprises avelumab formulated for IV infusion and administration at a dose of 200 mg. In some embodiments, a combination therapy as described herein comprises avelumab formulated for IV infusion and administration at a dose of 1200 mg. In some embodiments, a combination therapy as described herein comprises avelumab formulated for IV infusion and administration at a dose of 1600 mg. In some embodiments, a combination therapy as described herein comprises avelumab formulated for IV infusion and administration at a dose of 200-1600 mg. In some embodiments, a combination therapy as described herein comprises avelumab formulated for IV infusion and administration at a dose of 400-800 mg.

In some embodiments, a combination therapy as described herein comprises an anti-IL-2 antibody comprising a heavy chain variable region (VH) comprising heavy chain complementarity determining regions (HCDRs) HCDR1, HCDR2 and HCDR3 wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3. In some embodiments, a combination therapy as described herein comprises an anti-IL-2 antibody comprising a VH amino acid sequence as set forth in SEQ ID NO: 6. In some embodiments, a combination therapy as described herein comprises an anti-IL-2 antibody comprising a full length heavy chain having an amino acid sequence as set forth in SEQ ID NO:8.

In some embodiments, a combination therapy as described herein comprises an anti-IL-2 antibody comprising a light chain variable region (VL) comprising light chain complementarity determining regions (LCDRs) LCDR1, LCDR2 and LCDR3, said LCDR1 comprises the amino acid sequence of SEQ ID NO:4, said LCDR2 comprises the amino acid sequence of DAS, and said LCDR3 comprises the amino acid sequence of SEQ ID NO: 5. In some embodiments, a combination therapy as described herein comprises an anti-IL-2 antibody comprising a VL amino acid sequence as set forth in SEQ ID NO: 7. In some embodiments, a combination therapy as described herein comprises an anti-IL-2 antibody comprising a full length light chain having an amino acid sequence as set forth in SEQ ID NO: 9.

In some embodiments, a combination therapy as described herein comprises an anti-IL-2 antibody comprising a heavy chain variable region (VH) comprising heavy chain complementarity determining regions (HCDRs) HCDR1, HCDR2 and HCDR3 wherein said HCDR1 comprises the amino acid sequence of SEQ ID NO: 1, said HCDR2 comprises the amino acid sequence of SEQ ID NO:2, said HCDR3 comprises the amino acid sequence of SEQ ID NO:3, and a light chain variable region (VL) comprising light chain complementarity determining regions (LCDRs) LCDR1, LCDR2 and LCDR3, said LCDR1 comprises the amino acid sequence of SEQ ID NO:4, said LCDR2 comprises the amino acid sequence of DAS, and said LCDR3 comprises the amino acid sequence of SEQ ID NO:5. In some embodiments, a combination therapy as described herein comprises an anti-IL-2 antibody comprising a VH amino acid sequence as set forth in SEQ ID NO: 6 and a VL amino acid sequence as set forth in SEQ ID NO: 7. In some embodiments, a combination therapy as described herein comprises an anti-IL-2 antibody comprising a full length heavy chain having an amino acid sequence as set forth in SEQ ID NO:8 and a full length light chain having an amino acid sequence as set forth in SEQ ID NO:9.

In some embodiments, a combination therapy as described herein comprises an anti-IL-2 antibody comprising an IgG, IgA, IgM, IgE, IgD, a Fv, a scFv, a Fab, a F(ab′)₂, a minibody, a diabody, or a triabody. In some embodiments, a combination therapy as described herein comprises an anti-IL-2 antibody comprising a heavy chain comprising a mutation that reduces binding to an Fcγ receptor. In some embodiments, the mutation comprises a L234A mutation. In other embodiments, the mutation comprises a L235A mutation. In other embodiments, the mutation comprises both L234A and L235A mutations. In some embodiments, a combination therapy as described herein comprises an anti-IL-2 antibody that is an human IgG1 antibody with a LALA Fc mutation.

In some embodiments, a combination therapy comprises use of an anti-IL-2 antibody or composition thereof as described herein, and at least two checkpoint inhibitors or a composition thereof. In some embodiments, a combination therapy comprises use of an anti-IL-2 antibody or composition thereof and IL-2 as described herein, and at least two checkpoint inhibitors or a composition thereof.

In some embodiments, a combination therapy comprises a second composition comprising one or more checkpoint inhibitors, as described herein.

In some embodiments, a combination therapy comprises use of anti-IL-2 antibody BDG17.069 or composition thereof; and IL-2 as described herein; and a checkpoint inhibitor or a composition thereof as described herein. In some embodiments, a combination therapy comprises use of BDG17.069 or composition thereof; and a low dose of IL-2 as described herein; and a checkpoint inhibitor or a composition thereof as described herein. In some embodiments, a combination therapy comprises use of BDG17.069 or composition thereof; and a low dose of IL-2 as described herein; and a checkpoint inhibitor or a composition thereof, wherein said checkpoint inhibitor is selected from a PD-1 inhibitor or a PD-L1.

In some embodiments, a combination therapy comprises use of BDG17.069 or composition thereof; and a low dose of IL-2 as described herein; and a checkpoint inhibitor or a composition thereof, wherein said checkpoint comprises PD-L1. In some embodiments, a combination therapy comprises use of BDG17.069 or composition thereof; and a low dose of IL-2 (aldesleukin); and avelumab or a composition thereof. In certain embodiments of a combination therapy comprising BDG17.069 or composition thereof; and a low dose of IL-2 (aldesleukin); and avelumab or a composition thereof, the IL-2 is administered by subcutaneous injection. In some embodiments, the low dose of IL-2 is a loading dose. In other embodiments, the low dose of IL-2 is a booster dose.

In certain embodiments, the terms “IL-2” and “aldesleukin” may be used interchangeably having all the same meanings and qualities.

Thus, in some embodiments, a combination therapy comprises the use of BDG17.069 or composition thereof; and a loading dose of IL-2 as described herein; and a checkpoint inhibitor or a composition thereof, wherein said checkpoint comprises PD-L1. In some embodiments, a combination therapy comprises use of BDG17.069 or composition thereof; and a loading dose of IL-2 (aldesleukin); and avelumab or a composition thereof. In certain embodiments of a combination therapy comprising BDG17.069 or composition thereof; and a loading dose of IL-2 (aldesleukin); and avelumab or a composition thereof, the IL-2 is administered by subcutaneous injection. In some embodiments, the dose of IL-2 is a loading dose. In other embodiments, the dose of IL-2 is a booster dose.

In some embodiments, a combination therapy comprises use of BDG17.069 or composition thereof; and a booster dose of IL-2 as described herein; and a checkpoint inhibitor or a composition thereof, wherein said checkpoint comprises PD-L1. In some embodiments, a combination therapy comprises use of BDG17.069 or composition thereof; and a booster dose of IL-2 (aldesleukin); and avelumab or a composition thereof. In certain embodiments of a combination therapy comprising BDG17.069 or composition thereof; and a booster dose of IL-2 (aldesleukin); and avelumab or a composition thereof, the IL-2 is administered by subcutaneous injection.

In some embodiments of a combination therapy, the IL-2 administered comprises a low dose of IL-2. In some embodiments of a combination therapy, the IL-2 administered is by subcutaneous administration. In some embodiments of a combination therapy, the IL-2 administered comprises a low dose of IL-2 administered by subcutaneous administration.

In some embodiments of a combination therapy, an anti-IL-2 antibody and IL-2 are comprised in the same composition as a checkpoint inhibitor. In some embodiments, an anti-IL-2 antibody and IL-2 are comprised in different compositions from each other and from a checkpoint inhibitor. In some embodiments, an anti-IL-2 antibody, IL-2, and a checkpoint inhibitor are comprised in the same composition. In some embodiments, an anti-IL-2 antibody and IL-2 are comprised in a composition, and a checkpoint inhibitor is comprised in a different composition. In some embodiments, an anti-IL-2 antibody and a checkpoint inhibitor are comprised in a composition, and IL-2 is comprised in a different composition.

In some embodiments of a combination therapy, BDG17.069 and aldesleukin are comprised in the same composition as a PD-L1 checkpoint inhibitor. In some embodiments of a combination therapy, BDG17.069 and aldesleukin are comprised in the same composition as avelumab. In some embodiments, BDG17.069 and aldesleukin are comprised in different compositions from each other and from avelumab. In some embodiments, BDG17.069 and aldesleukin and avelumab are comprised in the same composition. In some embodiments, BDG17.069 and aldesleukin are comprised in a composition, and avelumab is comprised in a different composition. In some embodiments, BDG17.069 and avelumab are comprised in a composition, and aldesleukin is comprised in a different composition. In some embodiments of a combination therapy, the order of administration of an anti-IL-2 antibody or a composition thereof and a checkpoint inhibitor or a composition thereof, may be in any order. In some embodiments of a combination therapy, the order of administration of BDG17.069 or a composition thereof or a composition thereof and avelumab or a composition thereof, may be in any order. In some embodiments of a combination therapy, the order of administration of an anti-IL-2 antibody or a composition thereof, IL-2 or a composition thereof, and a checkpoint inhibitor or a composition thereof, may be in any order. In some embodiments of a combination therapy, the order of administration of BDG17.069 or a composition thereof, aldesleukin or a composition thereof, and avelumab or a composition thereof, may be in any order. For example, but not limited to the anti-IL-2 antibody may be administered prior to, concurrent with, or following administration of the checkpoint inhibitor. Similarly, a combination of an anti-IL-2 antibody and IL-2 may be administered prior to, concurrent with, or following administration of the checkpoint inhibitor. For example, but not limited to the BDG17.069 may be administered prior to, concurrent with, or following administration of the avelumab. Similarly, a combination of BDG17.069 and aldesleukin may be administered prior to, concurrent with, or following administration of the avelumab. In some embodiments, the anti-IL-2 antibody may be administered prior to, concurrent with, or following administration of the at least two checkpoint inhibitors. Similarly, a combination of an anti-IL-2 antibody and IL-2 may be administered prior to, concurrent with, or following administration of the at least two checkpoint inhibitors.

In some embodiments, administration of a combination therapy with a checkpoint inhibitor comprises concurrent administration of an anti-IL-2 antibody or a composition thereof and the checkpoint inhibitor. In some embodiments, administration of a combination therapy with a checkpoint inhibitor comprises concurrent administration of BDG17.069 or a composition thereof and avelumab. In some embodiments, administration of a combination therapy with a checkpoint inhibitor comprises concurrent administration of an anti-IL-2 antibody and IL-2, or composition(s) thereof and the checkpoint inhibitor. In some embodiments, administration of a combination therapy with a checkpoint inhibitor comprises concurrent administration of BDG17.069 and aldesleukin, or composition(s) thereof and avelumab. In some embodiments, administration of a combination therapy with a checkpoint inhibitor comprises prior administration of an anti-IL-2 antibody or a composition thereof before the checkpoint inhibitor. In some embodiments, administration of a combination therapy with a checkpoint inhibitor comprises prior administration of BDG17.069 or a composition thereof before the avelumab. In some embodiments, administration of a combination therapy with a checkpoint inhibitor comprises prior administration of an anti-IL-2 antibody and IL-2, or composition(s) thereof before the checkpoint inhibitor. In some embodiments, administration of a combination therapy with a checkpoint inhibitor comprises prior administration of BDG17.069 and aldesleukin, or composition(s) thereof before the avelumab. In some embodiments, administration of a combination therapy with a checkpoint inhibitor comprises later administration of an anti-IL-2 antibody or a composition thereof following administration of the checkpoint inhibitor. In some embodiments, administration of a combination therapy with a checkpoint inhibitor comprises later administration of BDG17.069 or a composition thereof following administration of the avelumab. In some embodiments, administration of a combination therapy with a checkpoint inhibitor comprises later administration of an anti-IL-2 antibody and IL-2, or composition(s) thereof following the administration of the checkpoint inhibitor. In some embodiments, administration of a combination therapy with a checkpoint inhibitor comprises later administration of BDG17.069 and aldesleukin, or composition(s) thereof following the administration of the avelumab.

In some embodiments, a combination therapy comprises use of a checkpoint inhibitor and an anti-IL-2 antibody comprising a heavy chain variable region having the sequence of SEQ ID NO:6. In some embodiments, a combination therapy comprises use of a checkpoint inhibitor and an anti-IL-2 antibody comprising a light chain variable region having the sequence of SEQ ID NO:7. In some embodiments, a combination therapy comprises use of a checkpoint inhibitor and an anti-IL-2 antibody comprising a heavy chain variable region and a light chain variable region having the sequences of SEQ ID NOs: 6 and 7. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs: 1-3 respectively. In some embodiments, a combination therapy comprises use of a checkpoint inhibitor and an anti-IL-2 antibody comprising a light chain variable domain comprising CDR1, CDR2 and CDR3 regions comprising amino acid sequences of SEQ ID NO: 4, DAS, and SEQ ID NO: 5, respectively. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs: 1-3, respectively, and a light chain variable domain comprising CDR1, CDR2, and CDR3 regions comprising amino acid sequences of SEQ ID NO: 4, DAS, and SEQ ID NO: 5, respectively.

In some embodiments, a combination therapy comprises use of a checkpoint inhibitor and an anti-IL-2 antibody comprising anti-IL-2 clone BDG17.069.

In some embodiments, a combination therapy comprises use of a checkpoint inhibitor; and an anti-IL-2 antibody comprising clone BDG17.069; and IL-2.

In certain embodiments, use of a combination therapy is for treating an unresectable locally advanced or metastatic NSCLC. In certain embodiments, use of a combination therapy is for treating an unresectable locally advanced or metastatic NSCLC that progressed after prior checkpoint inhibitor therapy. In certain embodiments, use of a combination therapy is for treating an unresectable locally advanced or metastatic NSCLC, wherein the NSCLC is PD-L1 positive NSCLC. In certain embodiments, use of a combination therapy is for treating an unresectable locally advanced or metastatic NSCLC, wherein the NSCLC is PD-1 positive NSCLC. In some embodiments, the NSCLC comprises a squamous NSCLC. In other embodiments, the NSCLC comprises a non-squamous NSCLC.

### Engineered Anti-IL-2 Antibodies

The present disclosure provides engineered anti-human IL-2 antibodies that bind human IL-2 with high affinity (e.g., 12.7 pM to 48 pM) to a pre-defined binding epitope. The antibodies bind to IL-2 in a manner that completely prevents CD25 binding, yet spares the binding of IL-2 to CD122, thereby modulating immune responses towards immune stimulation by directly activating and expanding effector cells without interacting with CD25− expressing cells (e.g., regulatory T-cells, short lived cytotoxic T-cells, pulmonary endothelial cells and vascular endothelial cells). Thus, the antibody/IL-2 complex would drive a robust immune response to clear a tumor by expanding and activating effector cells such as NK cells, central memory T cells and tumor-specific T-cells while inhibiting IL-2 activation induced cell death of the short lived CD25+ cytotoxic T-cells that are important for tumor clearance. The antibody/IL-2 complex would also decrease immunosuppression caused by the regulatory arm of the immune system. Moreover, the antibody/IL-2 complex prevent undesired interactions of IL-2 with vascular and pulmonary CD25− expressing cells, thereby preventing severe syndromes of IL-2 induced vascular leakage and IL-2 induced pulmonary edema. In some embodiments, the activity of the engineered anti-IL-2 antibodies described herein is dependent on the pre-defined epitope to which they are designed to bind.

In some embodiments, the IL-2 antibodies disclosed herein, block IL-2 binding to CD25. In some embodiments, the IL-2 antibodies disclosed herein binds IL-2 and prevent newly secreted endogenous IL-2 from binding to Tregs, effectively blocking the negative feedback loop of IL-2 to Tregs. In some embodiments, the IL-2 antibodies disclosed herein prevent Treg expansion. In some embodiments, the IL-2 antibodies disclosed herein block IL-2 binding to vascular endothelium. In some embodiments, the IL-2 antibodies disclosed herein block IL-2 binding to pulmonary endothelium. In some embodiments, the IL-2 antibodies disclosed herein block IL-2 binding to vascular and pulmonary endothelium.

In some embodiments, an anti-human anti-IL-2 antibody described herein inhibits binding of IL-2 with an IL-2 receptor alpha (IL-2 Ra, i.e., CD25) subunit and therefore inhibits binding to the trimer IL-2 Rαβγ receptor. In certain embodiments, anti-IL-2 antibodies that inhibit binding of IL-2 with a trimer IL-2 receptor (IL-2 Rαβγ) do not inhibit binding of IL-2 with the dimer IL-2 receptor (IL-2 Rβγ).

Targeting IL-2 to different cell populations can be used to either modulate the immune response toward immunosuppression or towards immune activation. The anti-human IL-2 antibodies disclosed herein are designed to bind with high affinity to an IL-2 epitope that blocks IL-2 binding to CD25. As a result, IL-2 is prevented from binding to short-lived CD8+ cytotoxic T cells or regulatory T cells that express high level of CD25 but is redirected to preferentially bind to effector T cells to stimulate enhanced immune response. Moreover, since IL-2 binding to CD25− expressing endothelial cells is also blocked, IL-2 induced pulmonary edema and vascular leaking would also be prevented.

In some embodiments, the present disclosure provides an anti-IL-2 antibody designed to enhance T cell immune response and to prevent severe edema symptoms of acute pneumonia induced by IL-2. The anti-IL-2 antibody binds specifically to human IL-2 with high affinity at a pre-defined epitope that blocks IL-2 binding to the alpha chain of the IL-2 receptors (CD25) while sparing binding to the main signaling beta chain and gamma chain complex of the receptor (CD122/CD132). Consequently, in the presence of such antibody, IL-2 would be directed to immune cells responsible for tumor clearance and away from cells that slow the immune response or cause the edema. The formation of this IL-2/antibody immunocomplex will direct IL-2 to bind and activate exclusively naive and memory T lymphocytes, NK cells, and Natural Killer T lymphocytes while preventing activation of regulatory T cells and apoptosis of short-lived CD25+ cytotoxic T effector cells. Altogether, the end result is an effective immune response, for example, tumor clearance. In addition, this treatment will prevent the toxicity caused by IL-2 binding to endothelial CD25 expressing cells. In other embodiments, treatment with the anti-IL-2 antibodies disclosed herein would be effective in preventing the toxicity caused by IL-2 binding to endothelial CD25 expressing cells, e.g., pulmonary edema, or IL-2-induced vascular leakage.

The cytokine IL-2 is critical for the expansion of T cells. However, in addition to its pro-stimulatory role IL-2 also induces some adverse side effects like lung edema and vascular leak syndrome through its binding to endothelium expressing the CD25 receptor.

In some embodiments, the present disclosure provides engineered anti-IL-2 antibodies resulting from introducing amino acid variations to a parent anti-IL-2 antibody. In some embodiments, the one or more of the amino acid variations are introduced in a CDR region. In other embodiments, the one or more amino acid variations are introduced within a framework (FR) region. In yet another embodiment, the amino acid variations are introduced to both the CDR region and the framework (FR) region. One of ordinary skill in the art would readily employ various standard techniques known in the art to introduce amino acid variations into an anti-IL-2 antibody and then test the resulting modified antibodies for any changes of binding to IL-2. While standard techniques may be used, the resultant binding pattern of the newly created antibodies is not predictable and must be analyzed to determine functionality.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In other embodiments, the VH and VL domains may be combined in a suitable buffer containing an enzyme and/or a cofactor which can promote dimerization of the VH and VL domains. In other embodiments, the VH and VL domains may be combined in a suitable vehicle that allows them to react with each other in the presence of a suitable reagent and/or catalyst.

In certain embodiments, the VH and VL domains may be contained within longer polypeptide sequences, which may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, CI). In some embodiments, the longer polypeptides may comprise multiple copies of one or both of the VH and VL domains generated according to the method disclosed herein; for example, when the polypeptides generated herein are used to form a diabody or a triabody.

In some embodiments, the Fc region comprises at least one mutation that reduces Fc-gamma binding, i.e., binding to a Fcγ receptor (FcγRs). In some embodiments, reduced binding is abolished binding, which binding to the Fcγ receptor is not detectable. In some embodiments, reduced binding reduces the binding affinity to a Fcγ receptor. In some embodiments, reduced binding reduces the on rate for binding to a Fcγ receptor. In some embodiments, reduced binding reduces the off rate of binding to a Fcγ receptor. In some embodiments, a mutation that reduces the Fc-gamma binding comprises L234A, L235A mutations, also known as LALA mutations. In some embodiments, a mutation that reduces the Fc-gamma binding comprises a P329G mutation in addition to the L234A, L235A mutations. In some embodiments, an antibody described herein comprises a heavy chain comprising a mutation that reduces binding to Fcγ receptor.

In some embodiments, the present disclosure provides an engineered (or modified) anti-IL-2 antibody, wherein the antibody comprises a heavy chain variable region having the sequence of SEQ ID NO: 6. In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure provides an engineered (or modified) anti-IL-2 antibody, wherein the antibody comprises a light chain variable region having the sequence of SEQ ID NO: 7. In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure provides an engineered (or modified) anti-IL-2 antibody, wherein the antibody comprises a heavy chain variable region and a light chain variable region having the sequences of on SEQ ID NOs: 6 and 7. In some embodiments, the engineered anti-IL-2 antibody comprises the sequences of SEQ ID NOs: 6 and 7.

In some embodiments, an isolated anti-IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein the HCDR1 comprises the amino acid sequence of SEQ ID NO: 1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of DAS, the LCDR3 comprises the amino acid sequence of SEQ ID NO:5.

In some embodiments, the VH and VL have the amino acid sequences wherein the VH comprises the amino acid sequence of SEQ ID NO:6, the VL comprises the amino acid sequence of SEQ ID NO:7.

In some embodiments, an antibody comprises a heavy chain sequence and a light chain sequence, said heavy chain sequence set forth in SEQ ID NO: 8 and said light chain sequence set forth in SEQ ID NO: 9. In some embodiments, an antibody comprising a heavy chain sequence and a light chain sequence, said heavy chain sequence set forth in SEQ ID NO: 8 and said light chain sequence set forth in SEQ ID NO: 9.

In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure also provides isolated polynucleotide sequence encoding a heavy chain variable region of an anti-IL-2 antibody, wherein the heavy chain variable region comprises the amino acid sequence of SEQ ID NO: 6. In other embodiments, the present disclosure also provides a vector comprising the above-mentioned polynucleotide sequences. In view of the amino acid sequences disclosed herein, one of ordinary skill in the art would readily construct a vector or plasmid to encode for the amino acid sequences. In other embodiments, the present disclosure also provides a host cell comprising the vector provided herein. Depending on the uses and experimental conditions, one of skill in the art would readily employ a suitable host cell to carry and/or express the above-mentioned polynucleotide sequences.

In some embodiments, the present disclosure also provides isolated polynucleotide sequence encoding a light chain variable region of an anti-IL-2 antibody, wherein the light chain variable region comprises the amino acid sequence of SEQ ID NO: 7. In other embodiments, the present disclosure also provides a vector comprising the above-mentioned polynucleotide sequences. In view of the amino acid sequences disclosed herein, one of ordinary skill in the art would readily construct a vector or plasmid to encode for the amino acid sequences. In other embodiments, the present disclosure also provides a host cell comprising the vector provided herein. Depending on the uses and experimental conditions, one of skill in the art would readily employ a suitable host cell to carry and/or express the above-mentioned polynucleotide sequences.

In view of the sequences for the heavy chain variable regions and light chain variable regions disclosed herein, one of ordinary skill in the art would readily employ standard techniques known in the art to construct an anti-IL-2 scFv. In some embodiments, polynucleotide sequences encoding for such anti-IL-2 scFv could have the sequence of SEQ ID NO: 10.

In certain embodiments, an isolated polynucleotide sequence disclosed herein, encoding the heavy chain variable region of an anti-IL-2 antibody, comprises a VH amino acid sequence set forth in the amino acid sequence of SEQ ID NO: 6. In some embodiments, a vector comprises the polynucleotide sequence of SEQ ID NO: 6. In some embodiments, a host cell comprising the vector comprising the polynucleotide sequence of SEQ ID NO: 6.

In certain embodiments, an isolated polynucleotide sequence disclosed herein, encoding the light chain variable region of an anti-IL-2 antibody, comprises a VL amino acid sequence as set forth in the amino acid sequence of SEQ ID NO: 7. In some embodiments, a vector comprises the polynucleotide sequence comprising the amino acid sequence of SEQ ID NO: 7. In some embodiments, a host cell comprises a vector comprising the polynucleotide sequence encoding the amino acid sequence of SEQ ID NO: 7. In some embodiments, an isolated polynucleotide sequence encodes an anti-IL-2 scFv, wherein the polynucleotide sequence encodes SEQ ID NO: 10. In some embodiments, a vector comprises an isolated polynucleotide sequence encodes an anti-IL-2 scFv, wherein the polynucleotide sequence encodes SEQ ID NO: 10. In some embodiments, a host cell comprises a vector comprising an isolated polynucleotide sequence encoding an anti-IL-2 scFv, wherein the polynucleotide sequence encodes SEQ ID NO: 10.

In other embodiments, the present disclosure also provides an isolated anti-IL-2 antibody, wherein the antibody comprises a heavy chain variable region having complementarity determining region (CDR) 1, CDR2 and CDR3. In some embodiments, the CDR1, CDR2 and CDR3 comprise the amino acid sequences of SEQ ID NOs: 1-3 respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In other embodiments, the present disclosure also provides an isolated anti-IL-2 antibody, wherein the antibody comprises a light chain variable region having complementarity determining region (CDR) 1, CDR2 and CDR3. In some embodiments, the CDR1, CDR2 and CDR3 comprise amino acid sequences of SEQ ID NO: 4, DAS, and SEQ ID NO: 5 respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In other embodiments, the present disclosure also provides an isolated anti-IL-2 antibody, wherein the antibody comprises a heavy chain variable region having complementarity determining region (CDR) 1, CDR2 and CDR3, and a light chain variable region having CDR1, CDR2 and CDR3. In some embodiments, the heavy chain CDR1, CDR2 and CDR3 comprise the amino acid sequences of SEQ ID NOs: 1-3, respectively. In some embodiments, the light chain CDR1, CDR2 and CDR3 comprise the amino acid sequences of SEQ ID NO: 4, DAS, and SEQ ID NO: 5, respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In some embodiments, an anti-IL-2 antibody as described herein is referred to as “BDG17.069” or “AU-007”, which may be used interchangeably with the term “anti-IL-2 antibody”, having all the same qualities and meanings. In addition, the term “imneskibart” may be used interchangeably with the terms “BDG17.069” or “AU-007” having all the same qualities and meanings, wherein imneskibart is the human monoclonal antibody also known as BDG17.069 or AU-007 with drug-like properties that provides advantages over non-natural biologics.

### Pharmaceutical Compositions

In some embodiments, disclosed herein are compositions for therapeutic use. In some embodiments, a composition described herein comprises an anti-IL-2 antibody as disclosed herein and a pharmaceutically acceptable carrier.

As used herein, the terms “composition” and pharmaceutical composition” may in some embodiments, be used interchangeably having all the same qualities and meanings. In some embodiments, disclosed herein is a pharmaceutical composition for the treatment of a condition or disease as described herein.

In some embodiments, disclosed herein are pharmaceutical compositions for use in a combination therapy.

In other embodiments, disclosed herein are compositions for use in treating an unresectable locally advanced or metastatic NSCLC.

In some embodiments, compositions comprise an anti-IL-2 antibody and a pharmaceutically acceptable carrier. In some embodiments, compositions comprise an anti-IL-2 antibody and IL-2, and a pharmaceutically acceptable carrier. In some embodiments, compositions comprise an anti-IL-2 antibody, IL-2, and an immune checkpoint inhibitor, and a pharmaceutically acceptable carrier. In some embodiments, compositions comprise an anti-IL-2 antibody, IL-2, and avelumab, and a pharmaceutically acceptable carrier.

In some embodiments, compositions comprise an anti-IL-2 antibody and a pharmaceutically acceptable carrier. In some embodiments, compositions comprise IL-2, and a pharmaceutically acceptable carrier. In some embodiments, compositions comprise an immune checkpoint inhibitor and a pharmaceutically acceptable carrier. In some embodiments, compositions comprise avelumab and a pharmaceutically acceptable carrier.

In some embodiments, an anti-IL-2 antibody and IL-2 are comprised in the same composition. In some embodiments, an anti-IL-2 antibody and IL-2 are comprised in different compositions. In some embodiments, an anti-IL-2 antibody and an immune checkpoint inhibitor are comprised in the same composition. In some embodiments, an anti-IL-2 antibody and an immune checkpoint inhibitor are comprised in different compositions. In some embodiments, an anti-IL-2 antibody and avelumab are comprised in the same composition. In some embodiments, an anti-IL-2 antibody and avelumab are comprised in different compositions. In some embodiments, an anti-IL-2 antibody, IL-2, and an immune checkpoint inhibitor are comprised in the same composition. In some embodiments, an anti-IL-2 antibody, IL-2 and an immune checkpoint inhibitor are comprised in different compositions. In some embodiments, an anti-IL-2 antibody, IL-2, and avelumab are comprised in the same composition. In some embodiments, an anti-IL-2 antibody, IL-2 and avelumab are comprised in different compositions.

In some embodiments, administration of a combination of an anti-IL-2 antibody and IL-2, or composition(s) thereof are concurrent. In some embodiments, administration of a combination of an anti-IL-2 antibody and IL-2, or composition(s) thereof comprises administration of an anti-IL-2 antibody or a composition thereof, prior to the IL-2 or a composition thereof. In some embodiments, administration of a combination of an anti-IL-2 antibody and IL-2, or composition(s) thereof comprises administration of an anti-IL-2 antibody or a composition thereof, following administration of the IL-2 or a composition thereof.

In some embodiments, administration of a combination of an anti-IL-2 antibody, IL-2, and an immune checkpoint inhibitor or composition(s) thereof are concurrent. In some embodiments, administration of a combination of an anti-IL-2 antibody, IL-2, and an immune checkpoint inhibitor or composition(s) thereof comprises administration of an anti-IL-2 antibody or a composition thereof, prior to the IL-2 and or the immune checkpoint inhibitor, or compositions thereof. In some embodiments, administration of a combination of an anti-IL-2 antibody, IL-2, and an immune checkpoint inhibitor or composition(s) thereof comprises administration of an anti-IL-2 antibody or a composition thereof, following administration of the IL-2 and or an immune checkpoint inhibitor, or a composition thereof.

In some embodiments, administration of a combination of an anti-IL-2 antibody, IL-2, and avelumab or composition(s) thereof are concurrent. In some embodiments, administration of a combination of an anti-IL-2 antibody, IL-2, and avelumab or composition(s) thereof comprises administration of an anti-IL-2 antibody or a composition thereof, prior to the IL-2 and or avelumab, or compositions thereof. In some embodiments, administration of a combination of an anti-IL-2 antibody, IL-2, and avelumab or composition(s) thereof comprises administration of an anti-IL-2 antibody or a composition thereof, following administration of the IL-2 and or avelumab, or a composition thereof.

In some embodiments, administering a loading dose of IL-2 or a composition thereof, comprises administering prior to, concurrent with, or following administration of the anti-IL-2 antibody, avelumab, or both. In certain embodiments of administering an anti-IL-2 antibody, IL-2, and or an immune checkpoint inhibitor, for example avelumab comprises administering a pharmaceutical composition comprising the anti-IL-2 antibody, the IL-2, and or the immune checkpoint inhibitor (e.g., avelumab), wherein the composition further comprises a pharmaceutically acceptable carrier. In some embodiments, each of the anti-IL-2 antibody, the IL-2, and the immune checkpoint inhibitor (e.g., avelumab) are comprised in separate compositions that further comprise a pharmaceutically acceptable carrier.

A skilled artisan would appreciate that a “pharmaceutical composition” may encompass a preparation of one or more of the active ingredients described herein with other chemical components such as physiologically suitable carriers and excipients. The purpose of a pharmaceutical composition is to facilitate administration of an active agent, for example but not limited to an antibody or a compound, to an organism.

In some embodiments, disclosed herein is a pharmaceutical composition for a therapy use treating a subject with a weakened immune system. In some embodiments, disclosed herein is a pharmaceutical composition for a therapy use treating a subject suffering from an unresectable locally advanced or metastatic NSCLC. In some embodiments, disclosed herein is a pharmaceutical composition for use as part of a combination therapy for treating a subject with a weakened immune system. In some embodiments, disclosed herein is a pharmaceutical composition for use as part of a combination therapy for use treating a subject suffering from an unresectable locally advanced or metastatic NSCLC.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

A skilled artisan would appreciate that an “excipient” may encompass an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences”, Mack Publishing Co., Easton, PA, latest edition, which is incorporated herein by reference.

In some embodiments, the composition as disclosed herein comprises a therapeutic composition. In some embodiments, the composition as disclosed herein comprises a therapeutic efficacy.

### Administration

In some embodiments, methods disclosed herein comprise administering compositions comprising an anti-IL-2 antibody as disclosed herein, in conjunction with a loading dose of IL-2. The method further comprises administering a booster dose of IL-2. In other embodiments, methods disclosed herein administer a combination therapy comprising compositions comprising an anti-IL-2 antibody as disclosed herein and a loading dose of IL-2. The method further comprises administering a booster dose of IL-2.

In some embodiments, methods disclosed herein comprise administering compositions comprising an anti-IL-2 antibody as disclosed herein and an immune checkpoint inhibitor as disclosed herein, in conjunction with a loading dose of IL-2. In some embodiments, the method further comprises administering a booster dose of IL-2. In other embodiments, methods disclosed herein administer a combination therapy comprising compositions comprising an anti-IL-2 antibody as disclosed herein and an immune checkpoint inhibitor as disclosed herein, and a loading dose of IL-2. In some embodiments, the method further comprises administering a booster dose of IL-2.

The anti-IL-2 antibody disclosed herein can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising the polypeptides disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Delivery can also be directed to any area of the respiratory system (e.g., lungs) via intubation. Alternatively, administration can be intratumoral, e.g., local or intravenous injection.

In certain embodiments, an anti-IL-2 antibody or composition thereof as described herein is administered intravenously (IV). In some embodiments, avelumab or composition thereof is administered intravenously (IV). In some embodiments, an immune checkpoint inhibitor or composition thereof is administered intravenously (IV). In some embodiments, low dose IL-2 or composition thereof is administered subcutaneously. In some embodiments, a loading dose of IL-2 or composition thereof is administered subcutaneously. In some embodiments, a booster dose of IL-2 or composition thereof is administered subcutaneously.

In some embodiments, IL-2 subcutaneous administration is at much lower doses and much less frequently than the approved regimen of intravenously administered aldesleukin. In some embodiments, wherein a subject receives both an anti-IL-2 antibody and IL-2, the route of administration of the anti-IL-2 antibody is by intravenous injection and the route of administration of the IL-2 is by subcutaneous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

In some embodiments, where an anti-IL-2 antibody and IL-2 are administered, they may be administered in the same composition. In some embodiments, where an anti-IL-2 antibody and IL-2 are administered, they may be administered in separate compositions. In some embodiments, where an anti-IL-2 antibody and an immune checkpoint inhibitor, for example avelumab are administered, they may be administered in the same composition. In some embodiments, where an anti-IL-2 antibody and an immune checkpoint inhibitor, for example avelumab are administered, they may be administered in separate compositions.

In some embodiments, IL-2 may be administered prior to, concurrent with, or following the step of administering the anti-IL-2 antibody. In some embodiments, IL-2 administration is prior to administering the anti-IL-2 antibody. In some embodiments, IL-2 administration is concurrent with administering the anti-IL-2 antibody. In some embodiments, IL-2 administration follows the step of administering the anti-IL-2 antibody.

In some embodiments, the immune checkpoint inhibitor or avelumab may be administered prior to, concurrent with, or following the step of administering the anti-IL-2 antibody. In some embodiments, the immune checkpoint inhibitor or avelumab administration is prior to administering the anti-IL-2 antibody. In some embodiments, the immune checkpoint inhibitor or avelumab administration is concurrent with administering the anti-IL-2 antibody. In some embodiments, the immune checkpoint inhibitor or avelumab administration follows the step of administering the anti-IL-2 antibody.

In some embodiments, IL-2 may be administered prior to, concurrent with, or following the step of administering the immune checkpoint inhibitor or avelumab. In some embodiments, IL-2 administration is prior to administering the immune checkpoint inhibitor or avelumab. In some embodiments, IL-2 administration is concurrent with administering the immune checkpoint inhibitor or avelumab. In some embodiments, IL-2 administration follows the step of administering the immune checkpoint inhibitor or avelumab.

In some embodiments, administration of an anti-IL-2 antibody comprises including a loading dose of IL-2 with the anti-IL-2 antibody. In some embodiments, administration of an anti-IL-2 antibody comprises a combination therapy, wherein anti-IL-2 antibody and an IL-2 loading dose is administered to a subject at regular intervals, while an IL-2 booster dose is administered to a subject when there is one or more objective signs of worsening tumor growth kinetics, which in some embodiments comprise (a) previously shrinking tumors becoming stable, (b) an increase in tumor markers, (c) an increase in tumor growth, (d) an increase in appearance of new tumor growth in a tumor that had previously been stable, (e) an increase in appearance of new tumor growth in a tumor that had previously decreased in size, or (f) any combination thereof.

In some embodiments, an anti-IL-2 antibody is administered weekly, bi-weekly (once every two weeks), or once every three weeks. In some embodiments, IL-2 is administered as a one-time loading” dose. In some embodiments, an immune checkpoint inhibitor is administered weekly, bi-weekly, once every three weeks, once every four weeks, once every five weeks, once every six weeks, once every seven weeks, or once every eight weeks. In some embodiments, avelumab is administered weekly, once every 2, 3, 4, 5, 6, 7, or 8 weeks. In some embodiments, the anti-IL-2 antibody, the IL-2, and the immune checkpoint inhibitor, for example avelumab, are administered independent of each other.

In some embodiments, therapeutic dosages of anti-IL-2 are administered at repeated administrations, e.g., of the same dose, over a period of months. In some embodiments, therapeutic dosages of anti-IL-2 are administered for up to 3 months. In some embodiments, therapeutic dosages of anti-IL-2 are administered for at least 3 months. In some embodiments, therapeutic dosages of anti-IL-2 are administered for up to 6 months. In some embodiments, therapeutic dosages of anti-IL-2 are administered for at least 6 months. In some embodiments, therapeutic dosages of anti-IL-2 are administered for up to 9 months. In some embodiments, therapeutic dosages of anti-IL-2 are administered for at least 9 months. In some embodiments, therapeutic dosages of anti-IL-2 are administered over a period of up to a year. In some embodiments, therapeutic dosages of anti-IL-2 are administered over a period of at least a year. In some embodiments, therapeutic dosages of anti-IL-2 are administered over a period of up to 2 years. In some embodiments, therapeutic dosages of anti-IL-2 are administered over a period of at least 2 years.

The duration of treatment with immune therapies is not well defined as the treatment duration for these immune therapies is often based on the response or lack of response to the drug/combination therapy. A skilled artisan would appreciate that a reasonable duration of treatment, in some embodiments, comprises about 2 years of therapy for patients who are receiving benefit from the treatment, for example but not limited to where the tumor(s) are stabilized or shrinking. In other embodiments, patients who progress immediately or only with short periods of tumor stabilization or shrinkage have a duration of treatment <2 years, most often prior to receiving 1 year of therapy.

In some embodiments, a combination therapy as described herein has a duration of about one year. In some embodiments, a combination therapy as described herein has a duration of less than one year. In some embodiments, a combination therapy as described herein has a duration of about two years. In some embodiments, a combination therapy as described herein has a duration of less than two years. In some embodiments, a combination therapy as described herein has a duration of about one-two years.

In some embodiments, the duration of administration of booster doses of IL-2 as part of a combination therapy varies between patient to patient. In some embodiments, a patient receives a single booster dose over a 1 year period. In some embodiments, a patient receives a single booster dose over a 2 year period. In some embodiments, a patient receives up to 6 booster doses over a 1 year period. In some embodiments, a patient receives up to 12 booster doses over a 2 year period. In some embodiments, a patient receives 1, 2, 3, 4, 5, or 6 booster doses over a 1 year period. In some embodiments, a patient receives 1, 2, 3, 4, 5, 6, 7, 8, 9, 10, 11, or 12 booster doses over a 2 year period. In some embodiments, a patient receives booster doses intermittently over a 6 month-two year period. In some embodiments, a patient receives booster doses intermittently over a 6-month-2 year period based on a clinician's review of one or more objective signs of worsening tumor growth kinetics.

In some embodiments, therapeutic dosages of avelumab are administered at repeated administrations, e.g., of the same dose, over a period of months. In some embodiments, therapeutic dosages of avelumab are administered for up to 3 months. In some embodiments, therapeutic dosages of avelumab are administered for at least 3 months. In some embodiments, therapeutic dosages of avelumab are administered for up to 6 months. In some embodiments, therapeutic dosages of avelumab are administered for at least 6 months. In some embodiments, therapeutic dosages of avelumab are administered for up to 9 months. In some embodiments, therapeutic dosages of avelumab are administered for at least 9 months. In some embodiments, therapeutic dosages of avelumab are administered over a period of up to a year. In some embodiments, therapeutic dosages of avelumab are administered over a period of at least a year.

In some embodiments, therapeutic dosages of the immune checkpoint inhibitor are administered at repeated administrations, e.g., of the same dose, over a period of months. In some embodiments, therapeutic dosages of the immune checkpoint inhibitor are administered for up to 3 months. In some embodiments, therapeutic dosages of the immune checkpoint inhibitor are administered for at least 3 months. In some embodiments, therapeutic dosages of the immune checkpoint inhibitor are administered for up to 6 months. In some embodiments, therapeutic dosages of the immune checkpoint inhibitor are administered for at least 6 months. In some embodiments, therapeutic dosages of the immune checkpoint inhibitor are administered for up to 9 months. In some embodiments, therapeutic dosages of the immune checkpoint inhibitor are administered for at least 9 months. In some embodiments, therapeutic dosages of the immune checkpoint inhibitor are administered over a period of up to a year. In some embodiments, therapeutic dosages of the immune checkpoint inhibitor are administered over a period of at least a year. In some embodiments, the immune checkpoint inhibitor is a PD-L1 immune checkpoint inhibitor. In other embodiments, the immune checkpoint inhibitor is a PD-1 immune checkpoint inhibitor.

In some embodiments, the dose of an anti-IL-2 antibody disclosed herein is between about 0.5 mg/kg and 12 mg/kg. In some embodiments, the dose of an anti-IL-2 antibody disclosed herein is about 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, and 12 mg/kg.

In some embodiments, the dose of an anti-IL-2 antibody disclosed herein is 0.5 mg/kg. In some embodiments, the dose of an anti-IL-2 antibody disclosed herein is 1.0 mg/kg. In some embodiments, the dose of an anti-IL-2 antibody disclosed herein is 1.5 mg/kg. In some embodiments, the dose of an anti-IL-2 antibody disclosed herein is 2.0 mg/kg. In some embodiments, the dose of an anti-IL-2 antibody disclosed herein is 2.5 mg/kg. In some embodiments, the dose of an anti-IL-2 antibody disclosed herein is 3.0 mg/kg. In some embodiments, the dose of an anti-IL-2 antibody disclosed herein is 3.5 mg/kg. In some embodiments, the dose of an anti-IL-2 antibody disclosed herein is 4.0 mg/kg. In some embodiments, the dose of an anti-IL-2 antibody disclosed herein is 4.5 mg/kg. In some embodiments, the dose of an anti-IL-2 antibody disclosed herein is 5.0 mg/kg. In some embodiments, the dose of an anti-IL-2 antibody disclosed herein is 5.5 mg/kg. In some embodiments, the dose of an anti-IL-2 antibody disclosed herein is 6.0 mg/kg. In some embodiments, the dose of an anti-IL-2 antibody disclosed herein is 6.5 mg/kg. In some embodiments, the dose of an anti-IL-2 antibody disclosed herein is 7.0 mg/kg. In some embodiments, the dose of an anti-IL-2 antibody disclosed herein is 7.5 mg/kg. In some embodiments, the dose of an anti-IL-2 antibody disclosed herein is 8.0 mg/kg. In some embodiments, the dose of an anti-IL-2 antibody disclosed herein is 8.5 mg/kg. In some embodiments, the dose of an anti-IL-2 antibody disclosed herein is 9.0 mg/kg. In some embodiments, the dose of an anti-IL-2 antibody disclosed herein is 9.5 mg/kg. In some embodiments, the dose of an anti-IL-2 antibody disclosed herein is 10.0 mg/kg. In some embodiments, the dose of an anti-IL-2 antibody disclosed herein is 10.5 mg/kg. In some embodiments, the dose of an anti-IL-2 antibody disclosed herein is 11.0 mg/kg. In some embodiments, the dose of an anti-IL-2 antibody disclosed herein is 11.5 mg/kg. In some embodiments, the dose of an anti-IL-2 antibody disclosed herein is 12.0 mg/kg. In some embodiments, the dose of an anti-IL-2 antibody disclosed herein is 12.5 mg/kg.

In some embodiments, the dose of IL-2 comprises a low dose. One skilled in the art would appreciate that a low dose of IL-2 may encompass dose level below those provided in studies currently known in the art. In certain embodiments, an IL-2 dose between about 10×10³ IU/kg-300×10³ IU/kg encompasses a low dose of IL-2. In certain embodiments, an IL-2 dose between about 10×10³ IU/kg-500×10³ IU/kg encompasses a low dose of IL-2. In certain embodiments, an IL-2 dose between about 15×10³ IU/kg-500×10³ IU/kg encompasses a low dose of IL-2. In certain embodiments, an IL-2 dose between about 45×10³ IU/kg-270×10³ IU/kg encompasses a low dose of IL-2. In certain embodiments, an IL-2 dose between about 45×10³ IU/kg-135×10³ IU/kg encompasses a low dose of IL-2. In certain embodiments, an IL-2 dose is 135×10³ IU/kg encompasses a low dose of IL-2.

In some embodiments, the dose of IL-2 is between about 10×10³ IU/kg-500×10³ IU/kg. In some embodiments, the dose of IL-2 is between about 10×10³ IU/kg-300×10³ IU/kg. In some embodiments, the dose of IL-2 is between about 15×10³ IU/kg-270×10³ IU/kg. In some embodiments, the dose of IL-2 is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500×10³ IU/kg. In some embodiments, the dose of IL-2 is about 15×10³ IU/kg. In some embodiments, the dose of IL-2 is about 45×10³ IU/kg. In some embodiments, the dose of IL-2 is about 135×10³ IU/kg. In some embodiments, the dose of IL-2 is about 270×10³ IU/kg. In some embodiments, the dose of IL-2 is about 300×10³ IU/kg. In some embodiments, the dose of IL-2 is about 400×10³ IU/kg. In some embodiments, the dose of IL-2 is about 500×10³ IU/kg. In some embodiments, the dose of IL-2 is 15×10³ IU/kg. In some embodiments, the dose of IL-2 is 45×10³ IU/kg. In some embodiments, the dose of IL-2 is 135×10³ IU/kg. In some embodiments, the dose of IL-2 is 270×10³ IU/kg. In some embodiments, the dose of IL-2 is 300×10³ IU/kg. In some embodiments, the dose of IL-2 is 400×10³ IU/kg. In some embodiments, the dose of IL-2 is 500×10³ IU/kg.

In some embodiments, methods described herein comprise administering avelumab to a subject at a dose of 240 mg. In other embodiments, methods described herein comprise administering avelumab to a subject at a dose of 120 mg. In other embodiments, methods described herein comprise administering avelumab to a subject at a dose of 360 mg. In other embodiments, methods described herein comprise administering avelumab to a subject at a dose of 600 mg. In other embodiments, methods described herein comprise administering avelumab to a subject at a dose of between about 120-600 mg. In other embodiments, methods described herein comprise administering avelumab to a subject at a dose of between about 500-1000 mg.

In some embodiments, methods described herein comprise administering an anti-IL-2 antibody to a subject at a dose of 9 mg/kg of the subject's body weight, IL-2 at a loading dose of 135,000 IU/kg of the subject's body weight, and IL-2 at a booster dose of 135,000 IU/kg of the subject's body weight. In some embodiments, the methods further comprise the step of administering avelumab at a dose of 800 mg to the subject.

In some embodiments, methods described herein comprise administering an anti-IL-2 antibody to a subject at a dose of 7 mg/kg of the subject's body weight-11 mg/kg of the subject's body weight, IL-2 at a loading dose of 100,000 IU/kg of the subject's body weight-270,000 IU/kg of the subject's body weight, and IL-2 at a booster dose of 100,000 IU/kg of the subject's body weight-270,000 IU/kg of the subject's body weight. In some embodiments, the methods further comprise the step of administering avelumab at a dose of 240 mg per subject-600 mg per subject to the subject.

In other embodiments, methods described herein comprise administering an anti-IL-2 antibody to a subject at a dose of 9 mg/kg of the subject's body weight, IL-2 at a loading dose of 135,000 IU/kg of the subject's body weight, and avelumab at a dose of 800 mg per subject. In some embodiments, the methods further comprise the step of administering IL-2 at a booster dose of 135,000 IU/kg of the subject's body weight to the subject.

In other embodiments, methods described herein comprise administering an anti-IL-2 antibody to a subject at a dose of 7 mg/kg of the subject's body weight-11 mg/kg of the subject's body weight, IL-2 at a loading dose of 100,000 IU/kg of the subject's body weight-270,000 IU/kg of the subject's body weight, and avelumab at a dose of 240 mg per subject-600 mg per subject. In some embodiments, the methods further comprise the step of administering IL-2 at a booster dose of 100,000 IU/kg of the subject's body weight-270,000 IU/kg of the subject's body weight to the subject.

In some embodiments when administering an anti-IL-2 antibody disclosed herein and a single loading dose of IL-2, the dose of anti-IL-2 antibody is 4.5 mg/kg and the dose of IL-2 is 15×10³ IU/kg. In some embodiments when administering an anti-IL-2 antibody disclosed herein and a single loading dose of IL-2, the dose of anti-IL-2 antibody is 4.5 mg/kg and the dose of IL-2 is 45×10³ IU/kg. In some embodiments when administering an anti-IL-2 antibody disclosed herein and a single loading dose of IL-2, the dose of anti-IL-2 antibody is 4.5 mg/kg and the dose of IL-2 is 135×10³ IU/kg. In some embodiments when administering an anti-IL-2 antibody disclosed herein and a single loading dose of IL-2, the dose of anti-IL-2 antibody is 4.5 mg/kg and the dose of IL-2 is 270×10³ IU/kg. In some embodiments when administering an anti-IL-2 antibody disclosed herein and a single loading dose of IL-2, the dose of anti-IL-2 antibody is 4.5 mg/kg and the dose of IL-2 is 300×10³ IU/kg. In some embodiments when administering an anti-IL-2 antibody disclosed herein and a single loading dose of IL-2, the dose of anti-IL-2 antibody is 4.5 mg/kg and the dose of IL-2 is 400×10³ IU/kg. In some embodiments when administering an anti-IL-2 antibody disclosed herein and a single loading dose of IL-2, the dose of anti-IL-2 antibody is 4.5 mg/kg and the dose of IL-2 is 500×10³ IU/kg.

In some embodiments when administering an anti-IL-2 antibody disclosed herein and a single loading dose of IL-2, the dose of anti-IL-2 antibody is 9.0 mg/kg and the dose of IL-2 is 15×10³ IU/kg. In some embodiments when administering an anti-IL-2 antibody disclosed herein and a single loading dose of IL-2, the dose of anti-IL-2 antibody is 9.0 mg/kg and the dose of IL-2 is 45×10³ IU/kg. In some embodiments when administering an anti-IL-2 antibody disclosed herein and a single loading dose of IL-2, the dose of anti-IL-2 antibody is 9.0 mg/kg and the dose of IL-2 is 135×10³ IU/kg. In some embodiments when administering an anti-IL-2 antibody disclosed herein and a single loading dose of IL-2, the dose of anti-IL-2 antibody is 9.0 mg/kg and the dose of IL-2 is 270×10³ IU/kg. In some embodiments when administering an anti-IL-2 antibody disclosed herein and a single loading dose of IL-2, the dose of anti-IL-2 antibody is 9.0 mg/kg and the dose of IL-2 is 300×10³ IU/kg. In some embodiments when administering an anti-IL-2 antibody disclosed herein and a single loading dose of IL-2, the dose of anti-IL-2 antibody is 9.0 mg/kg and the dose of IL-2 is 400×10³ IU/kg. In some embodiments when administering an anti-IL-2 antibody disclosed herein and a single loading dose of IL-2, the dose of anti-IL-2 antibody is 9.0 mg/kg and the dose of IL-2 is 500×10³ IU/kg.

In some embodiments when administering an anti-IL-2 antibody disclosed herein and a single loading dose of IL-2, the dose of anti-IL-2 antibody is 12 mg/kg and the dose of IL-2 is 15×10³ IU/kg. In some embodiments when administering an anti-IL-2 antibody disclosed herein and a single loading dose of IL-2, the dose of anti-IL-2 antibody is 12 mg/kg and the dose of IL-2 is 45×10³ IU/kg. In some embodiments when administering an anti-IL-2 antibody disclosed herein and a single loading dose of IL-2, the dose of anti-IL-2 antibody is 12 mg/kg and the dose of IL-2 is 135×10³ IU/kg. In some embodiments when administering an anti-IL-2 antibody disclosed herein and a single loading dose of IL-2, the dose of anti-IL-2 antibody is 12 mg/kg and the dose of IL-2 is 270×10³ IU/kg. In some embodiments when administering an anti-IL-2 antibody disclosed herein and a single loading dose of IL-2, the dose of anti-IL-2 antibody is 12 mg/kg and the dose of IL-2 is 300×10³ IU/kg. In some embodiments when administering an anti-IL-2 antibody disclosed herein and a single loading dose of IL-2, the dose of anti-IL-2 antibody is 12 mg/kg and the dose of IL-2 is 400×10³ IU/kg. In some embodiments when administering an anti-IL-2 antibody disclosed herein and a single loading dose of IL-2, the dose of anti-IL-2 antibody is 12 mg/kg and the dose of IL-2 is 250×10³ IU/kg.

In some embodiments when administering a combination therapy comprising an anti-IL-2 antibody disclosed herein and IL-2, wherein the dose of anti-IL-2 antibody is between about 0.1 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL-2 antibody is between about 0.1 mg/kg and 12 mg/kg anti-IL-2 antibody and the dose of IL-2 is between about 10×10³ IU/kg-500×10³ IU/kg. In some embodiments when administering a combination therapy comprising an anti-IL-2 antibody disclosed herein and IL-2, wherein the dose of anti-IL-2 antibody is between about 0.5 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL-2 antibody is between about 0.5 mg/kg and 12 mg/kg anti-IL-2 antibody and the dose of IL-2 is between about 10×10³ IU/kg-500×10³ IU/kg.

In some embodiments when administering a combination therapy comprising an anti-IL-2 antibody disclosed herein and IL-2, the dose of anti-IL-2 antibody is 0.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 500×10³ IU/kg.

In some embodiments when administering a combination therapy comprising an anti-IL-2 antibody disclosed herein and IL-2, the dose of anti-IL-2 antibody is 1.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 500×10³ IU/kg.

In some embodiments when administering a combination therapy comprising an anti-IL-2 antibody disclosed herein and IL-2, the dose of anti-IL-2 antibody is 4.5 mg/kg and the dose of IL-2 is about 10×10³ IU/kg-300 IU/kg. In some embodiments when administering a combination therapy comprising an anti-IL-2 antibody disclosed herein and IL-2, the dose of anti-IL-2 antibody is 4.5 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL-2 antibody is 4.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 500×10³ IU/kg. In some embodiments when administering a combination therapy comprising an anti-IL-2 antibody disclosed herein and IL-2, the dose of anti-IL-2 antibody is 9.0 mg/kg and the dose of IL-2 is about 10×10³ IU/kg-300 IU/kg. In some embodiments when administering a combination therapy comprising an anti-IL-2 antibody disclosed herein and IL-2, the dose of anti-IL-2 antibody is 9.0 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL-2 antibody is 9.0 mg/kg and the dose of IL-2 is 15×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 45×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 135×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 270×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 300×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 400×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 500×10³ IU/kg.

In some embodiments when administering a combination therapy comprising an anti-IL-2 antibody disclosed herein and IL-2, the dose of anti-IL-2 antibody is 12 mg/kg and the dose of IL-2 is 15×10³ IU/kg. In some embodiments when administering a combination therapy comprising an anti-IL-2 antibody disclosed herein and IL-2, the dose of anti-IL-2 antibody is 12 mg/kg and the dose of IL-2 is 45×10³ IU/kg. In some embodiments when administering a combination therapy comprising an anti-IL-2 antibody disclosed herein and IL-2, the dose of anti-IL-2 antibody is 12 mg/kg and the dose of IL-2 is 135×10³ IU/kg. In some embodiments when administering a combination therapy comprising an anti-IL-2 antibody disclosed herein and IL-2, the dose of anti-IL-2 antibody is 12 mg/kg and the dose of IL-2 is 270×10³ IU/kg. In some embodiments when administering a combination therapy comprising an anti-IL-2 antibody disclosed herein and IL-2, the dose of anti-IL-2 antibody is 12 mg/kg and the dose of IL-2 is 300×10³ IU/kg. In some embodiments when administering a combination therapy comprising an anti-IL-2 antibody disclosed herein and IL-2, the dose of anti-IL-2 antibody is 12 mg/kg and the dose of IL-2 is 400×10³ IU/kg. In some embodiments when administering a combination therapy comprising an anti-IL-2 antibody disclosed herein and IL-2, the dose of anti-IL-2 antibody is 12 mg/kg and the dose of IL-2 is 500×10³ IU/kg.

In some embodiments, the dose of IL-2 is considered low when compared with other therapies. In some embodiments, a low dose of IL-2 is between about 10×10³ IU/kg-500×10³ IU/kg. In some embodiments, a low dose of IL-2 is between about 10×10³ IU/kg-500×10³ IU/kg. In some embodiments, a low dose of IL-2 is between about 15×10³ IU/kg-500×10³ IU/kg. In some embodiments, a low dose of IL-2 is between about 45×10³ IU/kg-500×10³ IU/kg. In some embodiments, a low dose of IL-2 is equal to or less than about 500×10³ IU/kg. In some embodiments, a composition comprises an anti-IL-2 antibody comprising anti-IL-2 clone BDG17.069.

In some embodiments, methods described herein comprise administering an anti-IL-2 antibody to a subject once every 2 weeks, a loading IL-2 dose administered to the subject once, and a booster IL-2 dose administered to the subject as needed. In some embodiments, the booster IL-2 dose is administered to the subject once every 8 weeks. In some embodiments, the methods further comprise the step of administering avelumab to the subject once every 2 weeks.

In some embodiments, methods described herein comprise administering an anti-IL-2 antibody administered to a subject once every 1-4 weeks, a loading IL-2 dose administered to the subject once, and a booster IL-2 dose administered to the subject as needed. In some embodiments, the booster IL-2 dose is administered to the subject once every 4-16 weeks. In some embodiments, the methods further comprise the step of administering avelumab to the subject once every 1-4 weeks.

In other embodiments, methods described herein comprise administering an anti-IL-2 antibody to a subject once every 2 weeks, a loading IL-2 dose administered to the subject once, and avelumab to the subject once every 2 weeks. In some embodiments, the methods further comprise the step of administering IL-2 to the subject as needed. In some embodiments, the booster IL-2 dose is administered to the subject once every 8 weeks.

In other embodiments, methods described herein comprise administering an anti-IL-2 antibody to a subject once every 1-4 weeks, a loading IL-2 dose administered to the subject once, and an immune checkpoint inhibitor, for example avelumab to the subject once every 1-4 weeks. In some embodiments, the methods further comprise the step of administering IL-2 to the subject as needed in a booster dose. In some embodiments, the booster IL-2 dose is administered to the subject once every 4-16 weeks.

As used herein, the terms “booster” or “booster dose” may encompass an additional dose of a therapeutic agent for example IL-2, to enhance or prolong its effect. A skilled artisan would appreciate that a booster dose is a supplementary dose of a therapeutic compound, such as IL-2, given after a loading dose. In some embodiments, the booster dose is administered to provide additional IL-2 to the subject after blood levels of IL-2 are depleted over time. In some embodiments, the anti-IL-2 antibody binds to IL-2 and thereby blocks IL-2 from binding to Treg cells, eosinophils, and pulmonary and vascular endothelial cells expressing the high affinity trimeric receptor (CD25/CD132/CD122). Excess IL-2 that is not complexed to the anti-IL-2 antibody binds to the moderate affinity dimeric receptor (CD132/CD122), which favors the expansion and activation of Teff cells, NK cells, and Natural killer T (NKT) cells.

### Formulations

Pharmaceutical compositions disclosed herein comprising anti-IL-2 antibodies, or a combination of anti-IL-2 antibodies and IL-2, or checkpoint inhibitors, can be conveniently provided as sterile liquid preparations, e.g., isotonic aqueous solutions, suspensions, emulsions, dispersions, or viscous compositions, which may be buffered to a selected pH, Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the anti-IL-2 antibodies, or a combination of anti-IL-2 antibodies and IL-2, or checkpoint inhibitors, described herein and utilized in practicing the methods disclosed herein, in the required amount of the appropriate solvent with various amounts of the other ingredients, as desired. In other embodiments, each component is formulated separately. For example, a sterile injectable solution is prepared for the anti-IL-2 antibody, a sterile injectable solution is prepared for the IL-2, and a sterile injectable solution is prepared for the checkpoint inhibitor, for example but not limited to avelumab. Such formulations may be in admixture with a suitable carrier, diluent, or excipient such as sterile water, physiological saline, glucose, dextrose, or the like. The formulations can also be lyophilized. The formulations can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the formulations, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

In certain embodiments, the terms “pharmaceutical composition”, “composition”, and “formulation” may be used interchangeably having the same meanings and qualities.

The compositions or formulations described herein can be isotonic, i.e., they can have the same osmotic pressure as blood and lacrimal fluid. The desired isotonicity of the compositions as disclosed herein may be accomplished using sodium chloride, or other pharmaceutically acceptable agents such as dextrose, boric acid, sodium tartrate, propylene glycol or other inorganic or organic solutes. Sodium chloride may be preferred particularly for buffers containing sodium ions.

Viscosity of the compositions, if desired, can be maintained at the selected level using a pharmaceutically acceptable thickening agent. Methylcellulose may be preferred because it is readily and economically available and is easy to work with.

Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

In some embodiments, a composition is formulated to be at a pH between about pH 5.0-6.0. In some embodiments, a composition is formulated to be at a pH between about pH 5.0-7.0. In some embodiments, a composition is formulated to be at a pH between about pH 5.0-6.5. In some embodiments, a composition is formulated to be at a pH between about pH 5.0-5.5. In some embodiments, a composition is formulated to be at a pH between about pH 5.5-6.0. In some embodiments, a composition is formulated to be at a pH between about pH 5.5-6.5. In some embodiments, a composition is formulated to be at a pH between about pH 5.0. In some embodiments, a composition is formulated to be at a pH between about pH 5.5. In some embodiments, a composition is formulated to be at a pH between about pH 6.0. In some embodiments, a composition is formulated to be at a pH between about pH 6.5.

In some embodiments, a composition is formulated to be at a pH between about pH 5.0-6.0 and comprises a buffer. In some embodiments, the buffer comprises a pharmaceutically acceptable buffer. In some embodiments, the buffer comprises a histidine buffer or a citrate buffer. In some embodiments, the buffer comprises a histidine buffer. In some embodiments, the buffer comprises a citrate buffer.

In some embodiments, a composition is formulated to be at a pH between about pH 5.0-6.0 and comprises a buffer selected from a histidine buffer and a citrate buffer. In some embodiments, a composition is formulated to be at a pH between about pH 5.0-6.0 and comprises a histidine buffer. In some embodiments, a composition is formulated to be at a pH between about pH 5.0-6.0 and comprises a citrate buffer.

In some embodiments, a composition further comprises at least one of sucrose, methionine, or PS80, or any combination thereof. In some embodiments, a composition further comprises sucrose. In some embodiments, a composition further comprises methionine. In some embodiments, a composition further comprises PS80.

In some embodiments, a composition comprises an anti-IL-2 antibody as disclosed herein and is formulated to be at a pH between about pH 5.0-6.0 and comprises a buffer selected from a histidine buffer and a citrate buffer. In some embodiments, the composition further comprises IL-2.

Those skilled in the art will recognize that the components of the compositions or formulations should be selected to be chemically inert and will not affect the viability or efficacy of the early apoptotic cell populations as described herein, for use in the methods disclosed herein. This will present no problem to those skilled in chemical and pharmaceutical principles, or problems can be readily avoided by reference to standard texts or by simple experiments (not involving undue experimentation), from this disclosure and the documents cited herein.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

A skilled artisan would appreciate that the term “about”, may encompass a deviance of between 0.0001-5% from the indicated number or range of numbers. In some instances, the term “about”, may encompass a deviance of between 1-10% from the indicated number or range of numbers. In some instances, the term “about”, encompasses a deviance of up to 25% from the indicated number or range of numbers.

As used herein, the term “dose” may encompass, in some embodiments, a measured quantity of a therapeutic agent to be taken at one time. A skilled artisan would appreciate that administration of a dose encompasses providing the given measured quantity of the therapeutic agent to a subject.

### EXAMPLES

### Example 1

Phase 1/2 Clinical Trial for AU-007 with Recombinant Human IL-2 (Aldesleukin)

Engineered anti-IL-2 antibodies designed to redirect IL-2 activity toward effector T cells and NK cells while avoiding stimulation of regulatory T cells (Tregs) and CD25+ endothelial cells were generated and characterized as described in US-2024/0287169, which is incorporated herein by reference in its entirety.

AU-007 is a human mAb that binds IL-2 on its CD25 binding epitope. AU-007 bound IL-2 cannot bind trimeric (CD25, CD122, CD132) IL-2 receptors (IL-2R) on regulatory T cells (Tregs), vascular endothelium, or eosinophils, but IL-2's binding to dimeric IL-2R (CD122, CD132) on T effector (T eff) and NK cells is unhindered. AU-007 thus redirects IL-2 towards T eff and NK cell activation, while diminishing Treg activation and vascular leak. AU-007 uniquely redirects IL-2 generated from T eff cell expansion, converting a Treg-mediated autoinhibitory loop into an immune stimulating loop (as described in US-2024/0287169, which is incorporated herein by reference in its entirety). CDR sequences are defined according to IMGT numbering.

The heavy chain variable region and light chain variable region amino acid sequences of Clone 17.069 are presented in Table 2. Clone 17.069 comprises the LALA mutation (L234A, L235A mutations).

TABLE 2
VH and VL Amino Acid Sequences of Anti-IL-2 Clones.

| | Heavy Chain Variable | Light Chain Variable |
|---|---|---|
| Clone | Region (VH) | Region (VL) |
| 17.069 | QVQLVQSGAEVKKPGSSVKV | DIVMTQSPDSLAVSLGERATI |
| | SCKASGYSITDDLIHWVRQA | NCKSSQSLLRRGNQKNHLAWY |
| | PGQGLEWMGWIDPEDGETNY | QQKPGQPPKLLIYDASTGQSG |
| | AQKFQGRVTLTADTSTSTAY | VPDRFSGSGSGTDFTLTISSL |
| | MELSSLRSEDTAVYYCARSL | QAEDVAVYYCLQSYITPPTFG |
| | DSTWIYPFAYWGQGTLVTVS | AGTKVEIK |
| | S (SEQ ID NO: 6) | (SEQ ID NO: 7) |

The CDR sequences for Clone 17.069 is provided in Table 3.

TABLE 3
CDR Amino Acid Sequences of Anti-IL-2 Clones.

| | Heavy Chain | | | Light Chain | | |
|---|---|---|---|---|---|---|
| Clones | CDR1 | CDR2 | CDR3 | CDR1 | CDR2 | CDR3 |
| 17.069 | GYSI | IDPE | ARSLDST | QSLL | DAS | LQSYI |
| | TDDL | DGET | WIYPFAY | RRGN | | TPPT |
| | (SEQ | (SEQ | (SEQ ID | QKNH | | (SEQ |
| | ID | ID | NO: 3) | (SEQ | | ID |
| | NO: | NO: | | ID NO: | | NO: |
| | 1) | 2) | | 4) | | 5) |

The full length amino acid sequences for Clone 17.069 is provided in Table 4 below.

TABLE 4
Full-length Amino Acid Sequences of
Anti-IL-2 Clones.

| BGD-Clone | Heavy Chain (LALA) | Light Chain |
|---|---|---|
| BDG17.069 | QVQLVQSGAEVKKPGSSVKV | DIVMTQSPDSLAVSLGER |
| | SCKASGYSITDDLIHWVRQA | ATINCKSSQSLLRRGNQK |
| | PGQGLEWMGWIDPEDGETNY | NHLAWYQQKPGQPPKLLI |
| | AQKFQGRVTLTADTSTSTAY | YDASTGQSGVPDRFSGSG |
| | MELSSLRSEDTAVYYCARSL | SGTDFTLTISSLQAEDVA |
| | DSTWIYPFAYWGQGTLVTVS | VYYCLQSYITPPTFGAGT |
| | SASTKGPSVFPLAPSSKSTS | KVEIKRTVAAPSVFIFPP |
| | GGTAALGCLVKDYFPEPVTV | SDEQLKSGTASVVCLLNN |
| | SWNSGALTSGVHTFPAVLQS | FYPREAKVQWKVDNALQS |
| | SGLYSLSSVVTVPSSSLGTQ | GNSQESVTEQDSKDSTYS |
| | TYICNVNHKPSNTKVDKKVE | LSSTLTLSKADYEKHKVY |
| | PKSCDKTHTCPPCPAPEAAG | ACEVTHQGLSSPVTKSFN |
| | GPSVFLFPPKPKDTLMISRT | RGEC |
| | PEVTCWVDVSHEDPEVKFNW | (SEQ ID NO: 9) |
| | YVDGVEVHNAKTKPREEQYN | |
| | STYRVVSVLTVLHQDWLNGK | |
| | EYKCKVSNKALPAPIEKTIS | |
| | KAKGQPREPQVYTLPPSRDE | |
| | LTKNQVSLTCLVKGFYPSDI | |
| | AVEWESNGQPENNYKTTPPV | |
| | LDSDGSFFLYSKLTVDKSRW | |
| | QQGNVFSCSVMHEALHNHYT | |
| | QKSLSLSPGK | |
| | (SEQ ID NO: 8) | |

AU-007 may be formulated in 20 mM Histidine, 8% sucrose, 10 mM Methionine, 0.04% PS80, pH5.5.

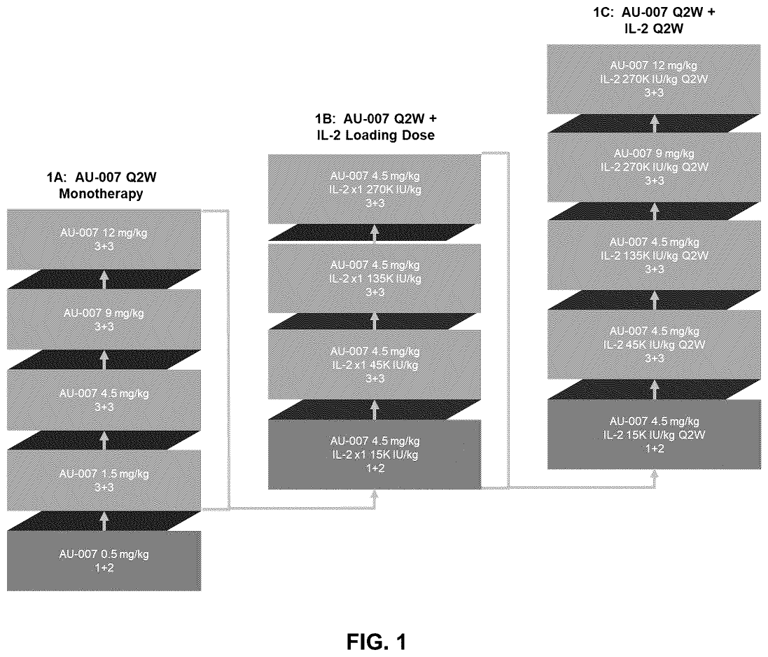

A clinical study was designed to test the efficacy of AU-007 with recombinant IL-2 (aldesleukin) in patients with cancer. In the study, 3 dose escalation arms were followed by cohort expansion (FIG. 1). Arm 1A evaluated escalating doses (0.5-12 mg/kg of said subject's body weight) of AU-007 (IV, once every 2 weeks [Q2W]). Arm 1B evaluated AU-007 Q2W+a single escalating loading low doses (15K-270K IU/kg of said subject's body weight) of aldesleukin administered subcutaneously (SC). Arm 1C evaluated AU-007+escalating low doses of SC aldesleukin, both Q2W. There were at least twenty solid tumor types allowed in the dose escalation arms. Cohort expansion Arm 2B evaluated 9 mg/kg of said subject's body weight AU-007+one loading dose of aldesleukin at a dose of 135K IU/kg of the subject's body weight. Tumor assessments were conducted at the end of each 8-week cycle.

In the B and C Arms (or B and C regimens) of the study, recombinant human IL-2 (aldesleukin) was administered subcutaneously, at much lower doses (15K IU/kg, 45K IU/kg, 135K IU/kg, or 270K IU/kg of said subject's body weight) and much less frequently than the approved regimen of intravenously administered aldesleukin, which is 600,000 IU/kg of said subject's body weight every 8 hours for up to 14 administrations.

In FIG. 1, the terms “3+3” and “1+2” refer to the size of the cohort at a given dose level. For 3+3, the first 3 enrolled patients were or will be administered AU-007 at the dose level that is noted. If no dose-limiting toxicities (DLTs) are seen, then administration may escalate to the next highest dose level. However, if any of the first 3 patients do have a dose-limiting toxicity that is drug-related, then an additional three more patients will receive treatment at that same dose level to see if they have DLTs before escalating. 1+2 follows the same principle-start with one patient, if DLTs are observed, add two more, or if no DLTs are observed in the first single patient, then dosages can be escalated.

Non-small cell lung cancer (NSCLC) was one of the at least 20 solid tumor histologies of patients enrolled in this study. For example, patients enrolled in the study included those suffering from NSCLC that has progressed during or following treatment with platinum-based chemotherapy and an anti-PDx therapy for unresectable locally advanced or metastatic disease. NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy).

In some cases, the NSCLC cancer may be unresectable locally advanced or metastatic cancer.

Adverse events were graded by the Common Terminology Criteria for Adverse Events (CTCAE). Efficacy evaluation was based on PD (Pharmacodynamic) markers of immune stimulation, total IL-2 (bound to AU-007+free IL-2) and objective responses.

Evaluation of efficacy is based on PD markers of immune stimulation, total IL-2 (bound to AU-007+free IL-2), and objective response.

Peripheral blood samples were taken prior to dosing on Day 1 and following dosing at 4 hours, and on Days 2, 3, 15, 29, and 43 of cycle 1 and pre-dose/Day 29 on all other cycles. Dosing with AU-007 was done via intravenous dosing by weight-based dosing (mg/kg). Proleukin® (aldesleukin) was dosed subcutaneously by weight-based dose (IU/kg). Whole blood was stained for CD4+ T cells, CD8+ T cells, CD4+ Tregs, NK cells, B cells, and monocytes. Samples were analyzed by flow cytometry using TruCount™ for absolute cell counts. Changes from baseline values were determined off absolute counts. In addition, differential hematology counts were taken on Days 1, 15, and 29 of all cycles (or as needed per treating physician's decision) for safety evaluation and to examine eosinophil levels (eosinophils express the IL-2 trimeric receptor). Serum was taken prior to dosing on Day 1 and at 2 hours, 6 hours, and Days 2, 3, 15 pre/post, and 6 hours, 29 pre/post, 43 pre/post, and subsequent Day 1 of each cycle at pre/post. Samples were analyzed for INF-γ (LOQ 31 fg/ml), IL-2 (LOQ 31 fg/ml), and sCD25 (10 fg/ml) using the ECL Mesoscale Dynamics platform.

### Example 2

### Clinical Trial Methodology

### Assessments of Subjects During the Screening Period (Pre-Treatment), During the Treatment Period, and Post-Treatment

The Screening Period was conducted within 28 days of Cycle 1 Day 1 and included the following procedures:
Physical examination including height and weight
Vital signs
Performance status
Medical history
Cancer history
Routine blood tests: comprehensive chemistry panel, special chemistry, hematology, coagulation, endocrine test
Tumor markers appropriate for tumor type (e.g., CA-125, CEA, CA19-9, LDH)
Routine urine test
Electrocardiogram
Urine or blood pregnancy test (women of childbearing potential)
CT or MRI scan or ultrasound or positron emission tomography (PET) scan if appropriate
Brain CT or MRI scan if appropriate
PD-L1 testing (if not previously tested)
Biopsy

The Treatment Period (Day 1 to Day 56 of each cycle) includes the following procedures:
Physical examination
Interim medical history
Study drug administration
Post-treatment observation
Vital signs
Performance status
Routine blood tests: comprehensive chemistry panel, special chemistry, hematology, coagulation, endocrine test
Tumor markers
Research blood tests: pharmacokinetics, cytokines and soluble biomarkers, flow cytometry, anti-drug antibodies
Routine urine test
Electrocardiogram
Urine or blood pregnancy test (women of childbearing potential)
CT or MRI scan or ultrasound or positron emission tomography (PET) scan if appropriate
Brain CT or MRI scan if appropriate.
Biopsy Cycle 2 Day 1 (optional in Dose Escalation)

### Post-Treatment Assessments

Following the last dose of study drug, all patients are followed by electronic (e.g., email) or telephone communication for safety and survival at Day 28 and Day 90, and then every 2 months for survival during a one-year Survival Follow-up Period.

### Safety

Dose-limiting toxicity criteria are defined as follows:

### Hematologic Dose-Limiting Toxicity

Grade 4 neutropenia lasting >7 days.
Grade 3 neutropenic fever.
Grade 3 thrombocytopenia with clinically significant bleeding.

### Hepatic Non-Hematologic Dose Limiting Toxicity

Grade 3 aspartate aminotransferase (AST) or alanine aminotransferase (ALT) increase >10×the upper limit of normal (ULN).
Grade 3 AST or ALT increase >5.0 to 10.0×ULN and not resolving to Grade 2 (i.e., >3.0 to 5.0×ULN) within 7 days and Grade 1 (i.e., >ULN to 3.0×ULN) within 14 days (unless patient enrolled with elevated transaminases secondary to metastases, then must resolve to baseline). Steroids must be tapered to ≤10 mg of prednisone or equivalent per day by Day 14.
Grade 3 bilirubin increase >5×ULN.
Grade 3 bilirubin increase >3.0 to 5.0×ULN and not resolving to Grade 2 (i.e., >1.5 to 3.0×ULN) within 7 days and Grade 1 (i.e., >ULN to 1.5×ULN) within 14 days. Steroids must be tapered to ≤10 mg of prednisone or equivalent per day by Day 14.
Any event meeting the Hy's law criteria (all 3 features must apply):
   AST and/or ALT >3×ULN.
   Concurrent elevation of total bilirubin >2×ULN.
   No alternative etiology can be identified for combination increases of AST/ALT and total bilirubin.

### Non-Hematologic Dose Limiting Toxicity

Non-hematologic DLTs are Grade ≥3 non-hematologic AEs with the following exceptions:
Grade 3 electrolyte abnormality lasting <72 hours without associated clinical complications and responds to therapy.
Grade 3 fever lasting <72 hours and not associated with hemodynamic instability.
Grade 3 nausea or vomiting resolving to ≤Grade 1 within 72 hours with or without medical intervention.
Grade 3 amylase and/or lipase elevations not associated with either clinical or radiographic evidence of pancreatitis requiring hospitalization.
Grade 3 diarrhea, constipation, abdominal pain, cramping, dyspepsia, or dysphagia resolving to Grade ≤1 within 72 hours with or without medical therapy.
Grade 3 fatigue resolving to Grade 1 within <7 days.
Grade 3 CLS.
Grade 3 IRR including cytokine release syndrome lasting ≤12 hours and responding to medical intervention. Grading is based on overall IRR event, and not grade of individual signs or symptoms including:
Fever, chills, nausea or vomiting, diarrhea, hypotension, hypertension, or tachycardia.
Grade 3 endocrinopathy controlled with hormone supplementation.
Grade 3 skin toxicity resolving to Grade ≤2 within 7 days with oral corticosteroids.
Grade 3 inflammatory reaction secondary to anti-tumor response (e.g., metastatic sites, lymph nodes) resolving to Grade ≤1 within 7 days.

### Grade 2 Non-Hematologic AEs May be Considered DLTs.

Grade 2 AEs that are prolonged inordinately, based upon the medical judgment of the Investigator, and/or lead to permanent discontinuation of AU-007 due to patient intolerance.

### Discontinuation Criteria Include:

Patient meets criteria for objective progression by modified Response Evaluation Criteria In Solid Tumors (RECIST) v1.1, i.e., progression that is confirmed by a second scan after receiving one more cycle of AU-007.
Occurrence of a DLT.
Sponsor, Investigator, or regulatory agency terminates the study.
Withdrawal of patient due to an adverse event or serious adverse event.
Patient experiences a medical condition that requires treatment with prohibited concomitant medication.
Withdrawal of patient consent.
Completion of protocol-defined therapy.
Pregnancy.

Patients who remain clinically stable (per RECIST definition below) and do not experience dose limiting toxicities (DLTs) or unacceptable toxicity during Cycle 1 may continue to receive additional 8-week treatment cycles until discontinuation criteria are met.

### Efficacy

Tumor assessment or evaluation e.g., by CT scan, MRI, PET scan, or ultrasound, is carried out at the end of each cycle, on Study Day 56. Tumor evaluation refers to imaging (CT scan, MRI, PET scan, or ultrasound) to measure tumors and assess for any new tumors—see Response Evaluation Criteria in Solid Tumors (RECIST) definition below.

### Response is Based on RECIST v1.1:

Complete Response: Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on the study (this includes the baseline sum if that is the smallest on study). In addition to the relative increase of 20%, the sum must also demonstrate an absolute increase of at least 5 mm. The appearance of one or more new lesions is also considered progression.
Stable Disease (SD): Neither sufficient shrinkage to qualify for PR nor sufficient increase to qualify for PD, taking as reference the smallest sum diameters while on study.

Exploratory biopsies are optionally conducted to evaluate the effect of the treatment on immune cell infiltration within the tumor. Biopsies are not conducted as part of determining a patient's response to treatment in conjunction with CT scans.

Patient blood samples for cytokines and soluble biomarkers were obtained per the following schedule:

### Cycle 1:

Day 1 pre-dose (prior to first study drug administration on a given day) and 2 and 6 hours post-dose
Day 2
Day 3
Day 15 pre-dose, end of infusion, and 6 hours post-dose
Day 29 pre-dose and end of infusion
Day 43 pre-dose and end of infusion

### Cycle 2:

Day 1 pre-dose and end of infusion
Day 15 pre-dose and end of infusion
Day 43 pre-dose and end of infusion

### Cycles 3 and Beyond:

Day 1 pre-dose and end of infusion.

### Example 3

### Combination Therapy Including AU-007, an IL-2 (Aldesleukin) Loading Dose, and Avelumab

Patients with unresectable locally advanced or metastatic, PD-L1-positive (tumor proportion score [TPS]≥1%), non-small cell lung cancer (NSCLC) not harboring an activating EGFR mutation or ALK rearrangement that has progressed during or following treatment with an anti-PDx (either PD-1 or PD-L1) with or without platinum-based chemotherapy are eligible for enrollment in the clinical trial of AU-007 in combination with aldesleukin and avelumab (“Part 3” of the study). Specifically, the combination therapy is for patients with unresectable locally advanced PD-L1-positive (tumor proportion score [TPS]≥1%) or metastatic PD-L1-positive (tumor proportion score [TPS]≥1%) NSCLC that does not contain an EGFR or ALK mutation and that has progressed during or following treatment with an anti-PDx (either PD-1 or PD-L1) with or without platinum-based chemotherapy.

Unresectable locally advanced tumors indicate tumors that cannot be resected and have spread locally (compared to metastatic tumors that have spread from the original location to another part of the body).

For the combination therapy of: i) AU-007; ii) aldesleukin (IL-2); and iii) avelumab; AU-007 and aldesleukin (IL-2) loading dose were/are administered at the recommended Phase 2 dose (RP2D) dose (9 mg/kg AU-007 of a subject's body weight (administered intravenously (IV) once every two weeks (Q2W)) plus a loading dose of 135,000 IU/kg aldesleukin (IL-2) of a subject's body weight (administered SC). Avelumab was/is administered IV at a dose of 800 mg with the initial dose of AU-007 plus aldesleukin and then Q2W. Safety run-in studies were performed with the RP2D−1 with 45K IU/kg IL-2 SC before escalating to the RP2D (described throughout in safety cohorts).

Booster IL-2 doses will be administered if assessment at the time of tumor evaluation warrants (FIG. 2—assessment occurs at tumor evaluation [diamond]). Assessment for administration of an IL-2 booster dose includes but is not limited to if tumor volume is unchanged, if previously shrinking tumors are stable, if there is an increase in tumor markers, if there is an increase in tumor growth, if new tumor growth is detected in a tumor that had previously been stable or had decreased in size, metastasis, or any combination thereof. An IL-2 booster dose would be administered subcutaneously.

FIG. 2 provides a timeline of the treatment and tumor assessment cycles for Study 3, which examines the effect of combination treatment of AU-007, aldesleukin, and avelumab. Cycles were/are 8 weeks (56 days). AU-007 was/is administered 4 times in a cycle (Day 1, 15, 29, 43—see black arrow); Aldesleukin was/is administered as a loading dose once in Cycle 1 (Day 1—see ellipse); and avelumab was/is administered 4 times in a cycle (Day 1, 15, 29, 43—see rectangle).

For patients who continued to subsequent cycles, they began with AU-007 and avelumab on Cycle 2 Day 1, then AU-007 administered with avelumab on Days 15, 29, and 43 of Cycle 2; and so on.

Tumor evaluation (FIG. 2, black diamond) was/is carried out at the end of a cycle. Tumor evaluation refers to imaging (CT scan, MRI, PET scan, or ultrasound) to measure tumors and assess for any new tumors—see RECIST definition in Example 2.

Biopsy (FIG. 2, 4-point star) was/is done as an exploratory objective to evaluate immune-modulating effect of drug within tumor by evaluating immune cell infiltration. They are not done as part of determining a patient's response to treatment in conjunction with CT scans.

The Dose-Limiting Toxicity (DLT) period lasted 4 weeks and informed dose escalation decisions. All patients treated in the safety run-in were followed for 4 weeks and their safety assessed before moving to the next dose level.

Patients who remained/remain clinically stable (per RECIST definition in Example 2) and did/do not experience DLTs or unacceptable toxicity during Cycle 1 continued/may continue to receive additional 8-week treatment cycles with AU-007 plus avelumab until discontinuation criteria as disclosed in Example 2 are met.

The study began with a Screening Period within 28 days of Cycle 1 Day 1 during which the Screening Period procedures described in Example 2 were conducted. The next period of the study was the Treatment Period from Day 1 to Day 56 of each cycle, during which the Treatment Period procedures described in Example 2 were conducted. Finally, Post-Treatment Assessments were conducted following the last dose of study drug. For Post-Treatment Assessments, all patients were followed by electronic (e.g., email) or telephone communication for safety and survival at Day 28 and Day 90, and then every 2 months for survival during a one-year Survival Follow-up Period.

### Safety Run-In Cohorts

An initial safety run-in of the combination therapy was conducted as a 3+3 dose escalation cohort (Table 5) in the Dose Limiting Toxicity (DLT) Evaluation Period (FIG. 2). AU-007+aldesleukin in combination with avelumab was evaluated starting with the RP2D−1 of AU-007+aldesleukin (Cohort 1 in Table 5 2^{nd} row), in which an IL-2 loading dose of 45,000 IU/kg was used as well as 9 mg/kg AU-007 IV Q2W, and 800 mg avelumab IV Q2W.

TABLE 5
Part 3 Safety Run-In Dose Levels

| Cohort | Dose Evaluated | | |
|---|---|---|---|
| (n) | Aldesleukin | AU-007 | avelumab |
| Dose | Loading Dose | Q2W | Q2W |
| Description | (IU/kg) | (mg/kg) | (mg) |
| −1 | 15,000 | 9 | 800 |
| (3 + 3) | | | |
| RP2D −2 | | | |
| + Avelumab | | | |
| 1 | 45,000 | 9 | 800 |
| (3 + 3) | | | |
| RP2D −1 | | | |
| + Avelumab | | | |
| 2 | 135,000 | 9 | 800 |
| (3 + 3) | | | |
| RP2D | | | |
| + Avelumab | | | |

### 3+3 Dose Escalation Cohort: Avelumab

If a DLT as defined in Example 2 was observed during the ‘DLT evaluation period’ (FIG. 2) in any of the initial 3 patients enrolled, 3 patients were added. If two or more DLTs were observed in the initial 6 patients, then the RP2D−2 (Cohort-1) were evaluated in combination with avelumab using an IL-2 loading dose of 15,000 IU/kg.

If no DLTs were observed at the RP2D−1 dose level, a cohort of 3 patients were evaluated at the RP2D+avelumab (Cohort 2) as described for the first cohort.

If no further DLTs were observed, then the Cohort Expansion proceeded with the AU-007+aldesleukin at the recommended Phase 2 dose (RP2D) dose (9 mg/kg AU-007 (administered IV Q2W) plus a loading dose of 135,000 IU/kg aldesleukin (administered SC), and avelumab. Avelumab was/is administered IV at a dose of 800 mg with the initial dose of AU-007 plus aldesleukin and then Q2W.

Avelumab dose escalation decisions were driven by clinical safety and all available pharmacokinetic (PK) and pharmacodynamic (PD) data during the dose limiting toxicity (DLT) Evaluation Period, defined as the time between the day of the initial dose up to the day of the 3^{rd} dose administration of AU-007 (planned Study Day 29 of Cycle 1; FIG. 2).

No DLTs were observed in Cohorts 1 and 2. Therefore, Cohort-1 was not initiated. Upon completion of the safety run-in, enrollment of the Cohort Expansion at the RP2D plus avelumab was initiated and is ongoing.

### Cohort Expansion

Patients enrolled in the Part 3 Cohort expansion receive the RP2D dose of 9.0 mg/kg AU-007 Q2W plus a loading dose of 135,000 IU/kg aldesleukin in combination with 800 mg avelumab Q2W as described in FIG. 2.

Patients who remain clinically stable (per RECIST definition in Example 2) and did not experience DLTs or unacceptable toxicity during Cycle 1 may continue to receive additional 8-week treatment cycles with AU-007 plus avelumab until discontinuation criteria as defined in Example 2 are met. Tumor assessments occur on Study Day 56 of each cycle. Response is based on RECIST v1.1 as described in Example 2.

An additional dose or doses of 135,000 IU/kg aldesleukin was administered at Investigator and Sponsor discretion based on objective tumor findings and pharmacodynamic data in patients who are tolerating treatment and are clinically stable as follows:
1. Aldesleukin administered with each cycle (Q8W, Day 1 of cycle) until objective tumor shrinkage is observed on radiologic imaging or physical exam;
2. Aldesleukin administered with objective signs of worsening tumor growth kinetics: e.g., previously shrinking tumors becoming stable, or with new tumor growth.
   Preliminary Results of AU-007 in Combination with Single-Dose Aldesleukin and Avelumab

### Cohort 1 (RP2D−1): 5 Patients Enrolled

1 patient did not complete DLT period and was replaced. No DLT were detected at the observed period.
1 patient discontinued at the end of Cycle 2 with 14% increase in target lesions.
1 patient continues on study in Cycle 4. The patient had a 27% decrease in target lesion at the end of Cycle 1, 45% decrease in target lesion at the end of Cycle 2, and a 36% decrease in target lesion at the end of Cycle 3. A new brain lesion was noted at the end of Cycle 2, which was treated with radiotherapy.
1 patient discontinued at Cycle 2 Day 1 with a 5% increase in target lesion.
1 patient had a 9% increase in target lesions at the end of Cycle 1 and continued to Cycle 2.
   Cohort 2 (RP2D) of Safety Run-In Cohorts: 3 Patients Enrolled. These Patients Were not Treated in Cohort 1 (RP2D−1) Before this Treatment.
1 patient discontinued at end of Cycle 1 with a 46% increase in target lesions
1 patient discontinued at end of Cycle 1 with an 18% increase in target lesions
1 patient is ongoing in Cycle 2, with a 10% decrease in target lesion at the end of Cycle 1; new brain mets noted.

### Cohort Expansion (RP2D): 3 Patients Enrolled to Date

1 patient had an 18% increase in target lesions at the end of Cycle 1 and discontinued.
1 patient is ongoing in Cycle 2, with a 28% increase in target lesions at the end of Cycle 1 and received an IL-2 boost on Cycle 2 Day 1.
1 patient had a partial response (43% decrease in target lesions) at the end of Cycle 1 and continues in Cycle 2.

Cohorts 1 and 2 were the safety run-in cohorts. Patients in any cohort, including safety run-in and expansion, can receive an IL-2 boost.

### Example 4

AU-007, a Human Monoclonal Antibody (mAb) that Binds to IL-2 and Inhibits CD25 Binding, Plus Low-Dose Aldesleukin and a Checkpoint Inhibitor (Avelumab): Phase 2 Update on Non-Small Cell Lung Cancer (NSCLC)

### Background

AU-007 is a monoclonal antibody (mAb) that binds to the interleukin-2 (IL-2) cytokine at an epitope inhibiting its interaction with CD25 on the IL-2 receptor while allowing engagement with the dimeric receptor complex, CD122/CD132. Thus AU-007 bound IL-2 expands Teff and NK cell populations (dimeric IL-2R) without expanding Tregs (trimeric IL-2R). Avelumab is an anti-PD-L1 mAb containing an active fragment crystallizable Fc region. Avelumab can potentially initiate antibody-dependent cell-mediated cytotoxicity mediated by NK cells, thus bringing innate and adaptive immunity against a tumor.

### Methods

The study describes an ongoing Ph2 expansion cohort (cohort 3) evaluating 8-week cycles of the recommended Ph2 dose (RP2D) of 9 mg/kg AU-007 IV Q2W+one subcutaneous (SC) 135K IU/kg aldesleukin dose on Day 1, plus the checkpoint inhibitor, avelumab. Cohort 3 evaluates PD-L1-positive (≥1%) NSCLC that has progressed on anti-PD-1/L1±chemotherapy at RP2D+avelumab.

### Results

Eight patients enrolled as of 30 May 2025 for the combination of AU-007+IL-2 with avelumab. AU-007+aldesleukin was well-tolerated in Ph1 with no dose-limiting toxicity (data not shown).

Early NSCLC activity was seen in a Ph1 patient who failed prior anti-PD-L1 therapy and received AU-007 Q2W monotherapy with a 14% tumor reduction (data not shown). Six NSCLC patients received avelumab plus the RP2D−1 (four patients: one with 45% target lesion reduction, two with SD, one pending evaluation) or the RP2D (two patients: one with progressive disease, one pending evaluation) (data not shown).

The most common treatment-related adverse events were Grade 1/2 fatigue, chills, pyrexia, infusion-related reaction, anemia, diarrhea, hypotension, and nausea.

### Conclusions

AU-007+low-dose SC aldesleukin+avelumab exhibits early signs of activity in the NSCLC cohort. The Ph2 cohort evaluating NSCLC+avelumab is ongoing.

### Example 5

Imneskibart (AU-007), a Human Monoclonal Antibody (mAb) that Binds IL-2 and Prevents CD25 Binding+Low-Dose Subcutaneous IL-2+Avelumab: Phase 2 Update on CPI-Refractory Non-Small Cell Lung Cancer (NSCLC)

### Background

Treatments activating effector cells against tumors are undermined by an autoinhibitory loop caused by endogenous IL-2 secreted from activated effector T cells (Teffs). Advantageously, Imneskibart (AU-007) can transform the IL-2 negative feedback loop into a positive feedback loop. In contrast, re-engineered IL-2 therapeutics cannot address the negative feedback loop, resulting in endogenous IL-2 stimulating regulatory T cell (Treg) expansion, and limiting efficacy.

### Methods

This is an ongoing Phase 1/2 open label dose escalation and expansion study—Phase 1 has been completed.

The recommended Phase 2 dose (RP2D) of imneskibart and low-dose subcutaneous (SQ) aldesleukin (IL-2) is imneskibart 9 mg/kg Q2W IV+SQ aldesleukin loading dose of 135K IU/kg. Administration of avelumab with RP2D of imneskibart and low-dose subcutaneous (SQ) aldesleukin (IL-2) is currently being/was evaluated in a Phase 2 expansion in patients with non-small cell lung cancer (NSCLC).

NSCLC cohorts evaluate the RP2D regimen plus avelumab (an anti-PD-L1 checkpoint inhibitor with Fc effector function for antibody-dependent cellular cytotoxicity (ADCC)) 800 mg IV, once every 2 weeks (Q2W) with a safety run-in (RP2D−1, aldesleukin 45K IU/kg) followed by cohort expansion.

Patient subjects have PD-L1 positive (PD-L1+) (tumor proportion score ≥1%) NSCLC, which has failed to beneficially respond to prior checkpoint inhibitor (CPI) therapy±chemotherapy

Efficacy is/was based on pharmacodynamic markers of immune stimulation and objective response; tumor assessments occurred at the end of each 8-week cycle. Patients can receive an additional SQ aldesleukin dose (booster dose) at the beginning of each 8-week cycle based on tumor growth kinetics observed on end-of-cycle scans.

### Enrolling Phase 2 in Post-CPI+/−Chemo-Failed, PD-L1 Positive Non-Small Cell Lung Cancer

Patients were/are being administered RP2D of 9 mg/kg imneskibart IV Q2W+one

135K IU/kg SQ aldesleukin loading dose+avelumab 800 mg IV Q2W. A boosting IL-2 dosing (135K IU/kg SQ aldesleukin) may be provided if tumor size is unchanged or increasing.

Enrollment goals and status: Avelumab+RP2D Imneskibart+SQ Aldesleukin Post PD-L1+ NSCLC n=20. Five (5) patients received 45K IU/kg aldesleukin in the completed safety run-in; 10 patients have been enrolled at the IL-2 RP2D (single loading dose 135 IU/kg aldesleukin-3 in a safety run-in and 7 in a Cohort Expansion). As of this time, 15 total patients have been enrolled and 6 are ongoing.

Patients tolerating study drug without confirmed objective pharmacodynamics (PD) can receive additional cycles of study drug until any one of the following conditions are met:
Patient meets criteria for objective progression by modified RECIST v1.1, i.e., progression that is confirmed by a second scan after receiving one more cycle of AU-007*.
Occurrence of a DLT*.
Sponsor, Investigator, or regulatory agency terminates the study.
Withdrawal of patient due to an Adverse Event (AE) or Serious Adverse Event (SAE).
   SAEs meet regulatory reporting requirements of serious:
   Death.
   Life-threatening (immediate risk of death).
   Inpatient hospitalization (>24 hrs) or prolongation of existing hospitalization (hospitalization for overnight observation after study drug is not counted as an SAE).
   Persistent or significant disability or incapacity.
   Congenital anomaly/birth defect.
   Important medical events that may not result in death, be life-threatening, or require hospitalization may be considered SAEs when, based upon medical judgment, may jeopardize the patient and may require medical or surgical intervention to prevent one of the outcomes listed in this definition.
Patient experiences medical condition that requires treatment with prohibited concomitant medication.
Withdrawal of patient consent.
Completion of protocol-defined therapy.
Pregnancy.
   * Patients meeting these criteria but otherwise experiencing substantial clinical benefit with acceptable toxicity based on Investigator opinion may continue therapy on a case-by-case basis.

### Avelumab Patient Summaries

### Safety Run-In RP2D−1 (45 IU/Kg Subject Body Weight SQ Aldesleukin Loading Dose)

### US04-0097

77-year-old male with NSCLC (adenocarcinoma) with target tumor lesions in the lung and lymph nodes, and non-target lesions of pleural effusion and brain.

The patient progressed on prior carboplatin and taxol, and anti-PD-1 and Avastin. November 2024, the patient the patient began treatment with the RP2D−1 imneskibart+aldesleukin in combination with avelumab 800 mg Q2W

The patient discontinued after 2 treatments due to disease progression, so was not evaluable for the 28-day DLT evaluation period and had to be replaced. The patient was also not evaluable for efficacy per RESICT as no end of treatment scan was performed.

### AU07-0100

68-year-old female with NSCLC (unknown subtype) with target tumor lesions in lung and lymph nodes, and non-target lesions in the lymph nodes, lung, and adrenal. The patient progressed on prior anti-PD-1+carboplatin+pemetrexed and prior docetaxel.

December 2024, the patient began treatment with the RP2D−1 imneskibart+aldesleukin in combination with avelumab 800 mg Q2W. The patient had stable disease in target lesions at the end of Cycle 1, and discontinued after 3 treatments in Cycle 2 with progressive disease (14% increase in target tumors and new liver lesion and pleural effusion)

### US04-0102

62-year-old male with NSCLC (squamous cell carcinoma) with a target tumor lesion in the pleural cavity, and non-target lesions in the lymph nodes, bone, and lung. The patient received prior anti-CTLA-4/anti-PD-1 and prior anti-PD-1.

January 2025, the patient began treatment with the RP2D−1 imneskibart+aldesleukin in combination with avelumab 800 mg Q2W. The patient had stable disease (5% increase) in target lesions at the end of Cycle 1, and discontinued after 1 treatment in Cycle 2 (withdrew consent) and died approximately one month later.

### US02-0116

69-year-old male with NSCLC (adenocarcinoma) with target tumor lesions in the liver, adrenal, and lymph nodes, and non-target lesions in the lung, lymph nodes, and bone. The patient progressed on prior anti-PD-1, pemetrexed, and carboplatin, and prior alectinib and lorlatinib.

May 2025, the patient began treatment with the RP2D−1 imneskibart+aldesleukin in combination with avelumab 800 mg Q2W. The patient had stable disease (9% decrease) in target lesions at the end of Cycle 1, and discontinued after 3 treatments in Cycle 2 with progressive disease.

### US01-0104

65-year-old male with NSCLC (adenocarcinoma) with a target tumor lesion in the lung, and non-target lesions in the lung, mediastinum, soft tissue, and bone. The patient progressed on prior paclitaxel and carboplatin; prior anti-PD-1, pemetrexed, and carboplatin; docetaxel; and gemcitabine.

January 2025, the patient began treatment with the RP2D−1 imneskibart+aldesleukin in combination with avelumab 800 mg Q2W. The patient had stable disease (27% decrease) in target lesions at the end of Cycle 1; partial response (45% decrease) in target lesions at the end of Cycle 2 with overall progressive disease due to a new brain lesions, which were irradiated. The patient continued to Cycle 3 with partial response in target lesions (36% decrease) and discontinued due to disease progression.

### Safety Run-In RP2D_(135,000 IU/Kg Subject Body Weight SQ Aldesleukin Loading Dose)

### AU03-0108

77-year-old male with NSCLC (squamous cell carcinoma) with target tumor lesions in the lung, chest wall, liver, and non-target lesions in the lymph nodes. The patient received prior carboplatin and paclitaxel, progressed on anti-PD-1, and progressed on docetaxel. February 2025, the patient began treatment with the RP2D imneskibart+aldesleukin in combination with avelumab 800 mg Q2W. The patient had disease progression (46% increase) in target lesions at the end of Cycle 1 and discontinued.

### US01-0114

66-year-old male with NSCLC (adenocarcinoma) with a target tumor lesions in the lung and non-target lesions in the bone and lung. The patient progressed on prior adjuvant pemetrexed and carboplatin, and had stable disease on palliative atezolizumab. May 2025, the patient began treatment with the RP2D imneskibart+aldesleukin in combination with avelumab 800 mg Q2W. The patient had stable disease (10% decrease) in target lesions with new brain lesions at the end of Cyle 1, which were irradiated. The patient continued to Cycle 2 with stable disease (10% decrease) and continues on study in Cycle 3.

### US02-0115

66-year-old male with NSCLC (squamous cell carcinoma) with target tumor lesions in the lung, liver, and kidney, and non-target lesions in the lung, bone, kidney, and lymph nodes. The patient had a complete response to prior adjuvant cisplatin and docetaxel; received prior carboplatin, taxol, and anti-PD-1; and prior carboplatin and gemcitabine.

May 2025, the patient began treatment with the RP2D imneskibart+aldesleukin in combination with avelumab 800 mg Q2W. The patient had stable disease (18% increase) in target lesions at the end of Cycle 1; per Investigator's discretion, the patient had progressive disease and was discontinued.

### Cohort Expansion RP2D (135,000 IU/Kg Subject Body Weight SQ Aldesleukin Loading Dose)

### AU07-0122

61-year-old male with NSCLC (squamous cell carcinoma) with a target tumor lesion in the mediastinum and a non-target lesion in the lung. The patient progressed on prior adjuvant carboplatin and paclitaxel and on prior adjuvant durvalumab.

July 2025, the patient began treatment with the RP2D imneskibart+aldesleukin in combination with avelumab 800 mg Q2W. The patient had progressive disease (28% increase) in target lesions at the end of Cycle 1 and received an IL-2 boost on Cycle 2 Day 1. The patient continues on study in Cycle 2.

### US02-0124

68-year-old female with NSCLC (adenocarcinoma) with two target tumor lesions in the lung, and non-target lesions in the lung, adrenal, and bone. The patient had a partial response to prior anti-PD-1, carboplatin, and pemetrexed and prior anti-PD-1 and pemetrexed; had stable disease following docetaxel and gemcitabine.

August 2025, the patient began treatment with the RP2D imneskibart+aldesleukin in combination with avelumab 800 mg Q2W. The patient had a partial response (43% decrease) in target lesions at the end of Cycle 1 and continues on study in Cycle 2.

### US02-0127

63-year-old male with NSCLC (adenocarcinoma) with a target tumor lesion in the lung and right lower posterior pleural nodule, and non-target lesions of right pleura and mediastinal, right hilar and infra hilar lymph nodes. The patient progressed after adjuvant anti-PD-1; progressed after an antibody-drug conjugate; and progressed after pemetrexed and anti-PD-1.

August 2025, the patient began treatment with the RP2D imneskibart+aldesleukin in combination with avelumab 800 mg Q2W

Efficacy data not available at time of analysis.

### Results

Preliminary Activity with Imneskibart+IL-2+Avelumab in Phase 2 in NSCLC Patients Who Failed Prior (Checkpoint Inhibitor) CPI±Chemotherapy

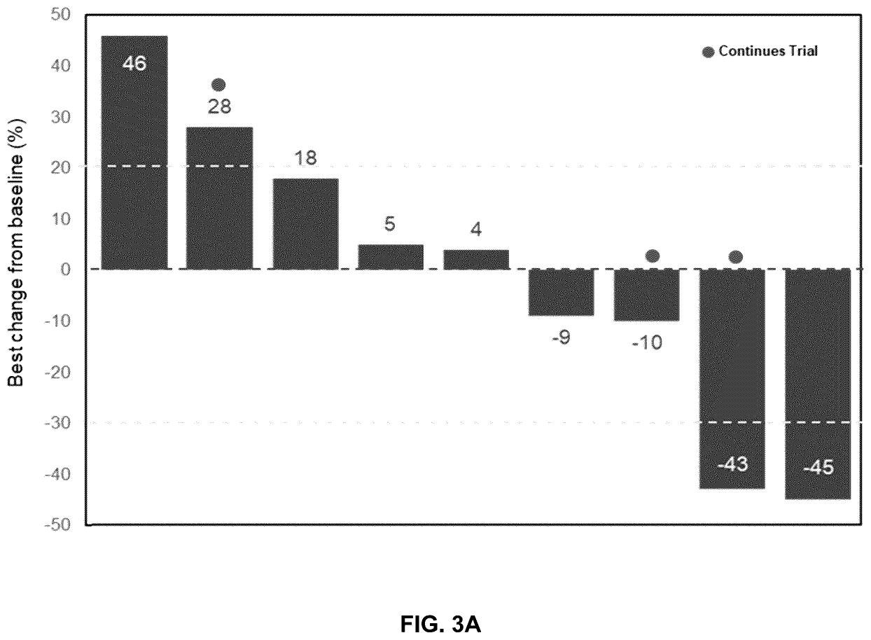

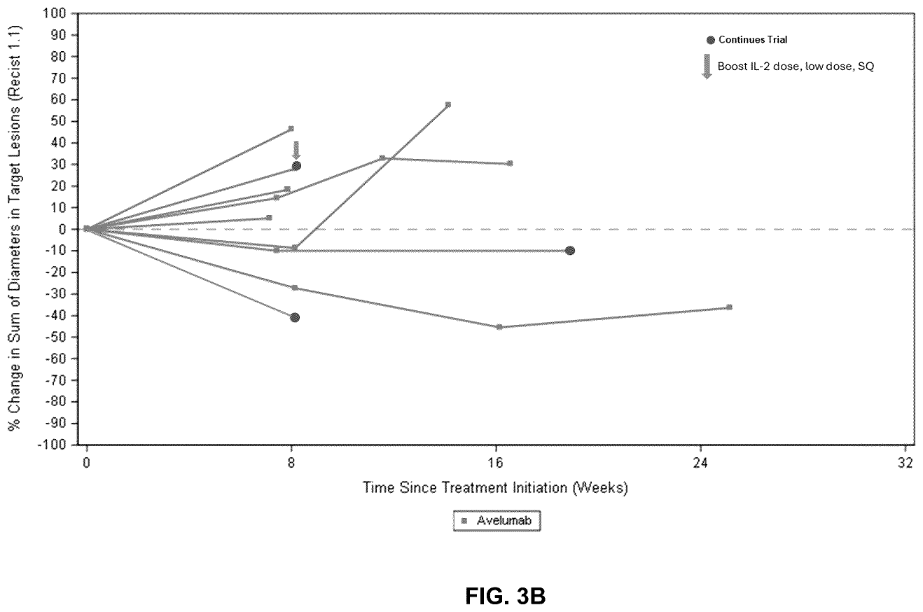

FIGS. 3A and 3B show preliminary activity of antitumor activity with AU-007+SQ loading dose of aldesleukin in combination with avelumab in patients with NSCLC who failed prior CPI therapy±chemotherapy. The Y-axis of FIG. 3A represents the best change in target lesion measurements from baseline. Five (5) of the patients in FIG. 3A received the RP2D dosage (9 mg/kg patient body weight AU-007, 135,000 IU/kg patient body weight IL-2, and 800 mg avelumab). Patients receiving the RP2D dosage+avelumab had increases of best change from the baseline of 46%, 28%, and 18%, and, and decreases of the best change from the baseline of 9%, 10%, 43%, and 45%. Those subjects continuing in the trial all received RP2D and are indicated by a filled circle. All ongoing patients are on RP2D.circle. The patient in FIG. 3B (patient AU07-0122) showing ~28% increased (+) change in target lesion sum of diameters received an IL-2 booster dose on Cycle 2 Day 1 (this was after the data extract for this chart). Patients AU07-0122 and US02-0124 are ongoing in the clinical trial and being treating with RP2D+avelumab.

In FIG. 3A, it can be seen that this patient's best change in a target lesion measurement from baseline was a decrease of ~10%. This patient is continuing in the clinical trial. Two of the patients treated with AU-007, IL-2, and avelumab are continuing on in the clinical trial.

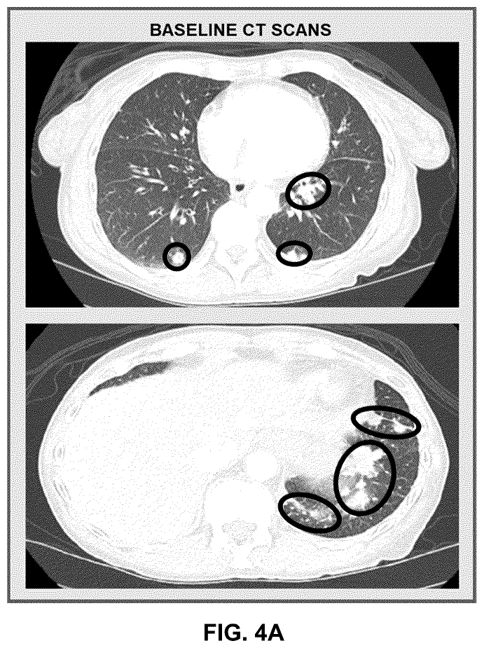

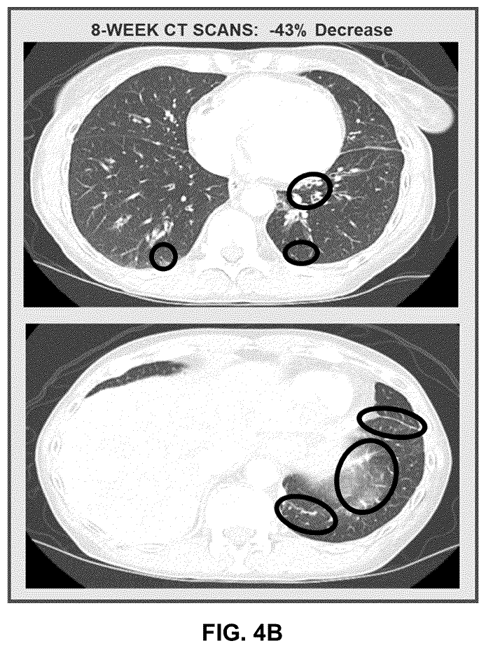

Significant tumor reduction was observed at 8 weeks (FIGS. 4A and 4B) in the pulmonary target lesion of a NSCLC patient who had previously progressed through prior anti-PD-1+chemotherapy treatments (US02-0124).

The patient is a 68-year-old woman with adenocarcinoma two target pulmonary lesions and non-target involvement of lung, adrenal, and bone. The patient progressed on prior carboplatin/anti-PD-1/pemetrexed and docetaxel/gemcitabine treatments. As of July 2025, the patient began treatment with RP2D imneskibart+aldesleukin (9 mg/kg of patient body weight imneskibart+135,000 IU/kg of patient body weight loading dose IL-2 loading dose) in combination with avelumab 800 mg Q2W (once every two weeks). Forty-three percent (43%) decrease in target lesion size was noted at end of Cycle 1; patient continues in Cycle 2. No booster dose was given at the start of Cycle 2.Cycle 2. No booster dose was given at the start of cycle 2.

### Adverse Event Summary in Phase 1 and 2: Mild and Tolerable Profile

The data presented in Tables 6A and 6B demonstrates that most drug-related adverse events (AEs) were mild, Grade 1 or 2, with imneskibart+SQ loading dose aldesleukin alone or in combination with avelumab. The “Imneskibart+IL-2” patients include subjects suffering from different solid cancers. The “Avelumab Combo” (Imneskibart+IL-2+avelumab) patients include subjects suffering from PD-L1 positive NSCLC, which has failed to beneficially respond to prior checkpoint inhibitor (CPI) therapy±chemotherapy.

TABLE 6A

| Low Rate of Drug Related SAEs and Grade 3/4 AEs¹ | |
|---|---|
| | Avelumab Combo² |
| | N = 11 |
| Event n, (%) | (# of patient (%) |
| Any AE | 11 (100) |
| Drug-Related AEs | 7 (64) |
| Drug-Related SAEs | 2 (18) |
| CRS | 0 |
| Infusion-related reaction | 0 |
| Pyrexia | 1 |
| Adrenal insufficiency | 1 |
| Injection site rash | 0 |
| Drug-Related Grade 3 or 4 AEs | 2 (18) |
| Lymphopenia | 1 |
| CRS | 1 |
| Anemia | 0 |
| Lipase elevation | 0 |
| Neutropenia | 0 |
| Maculopapular rash | 0 |
| Hypertension | 1 |
| Autoimmune encephalitis | 0 |

¹Data cutoff as of September 29, 2025.
²Imneskibart Q2W + IL-2 loading dose + avelumab

TABLE 6B
Most Drug Related AEs Tolerable and Easily Managed
Drug-Related Adverse Events in > 10% of
Patients

| Adverse | Avelumab Combo¹ |
|---|---|
| Event (n, %) | N = 11 |
| Pyrexia | 2 (18) |
| Fatigue | 2 (18) |
| Chills | 4 (36) |
| Infusion-related reaction | 2 (18) |
| Nausea | 2 (18) |
| Injection site reaction | 0 |
| Cytokine release | 1 (9) |
| syndrome | |
| Anemia | 2 (18) |
| Diarrhea | 2 (18) |
| Hypotension | 2 (18) |
| Fall | 0 |
| Dermatitis acneiform | 0 |

¹Imneskibart Q2W + IL-2 loading dose + avelumab.

Most drug-related adverse events (AEs) were mild, Grade 1 or 2, with imneskibart+SQ loading dose aldesleukin in combination with avelumab. The addition of avelumab to imneskibart+SQ loading dose aldesleukin did not increase the rate of drug related Grade 3 and 4 AEs in the emerging data. The most common Grade 3 or 4 AE was transient (3-7 days) lymphopenia that was not associated with adverse outcomes in any patient. Transient lymphopenia is a known effect of IL-2 treatment as lymphocytes traffic out of blood and into tissue.

It is expected that measurements of pharmacodynamic profile of CD8 T cells will show increases in CD8 T cells and CD8/Treg ratios, and corresponding decrease in Tregs. The NSCLC data is currently being collected.

### Conclusions

Early signal of activity was observed in the NSCLC patients whose tumors progressed on prior anti-PD-1/L1 therapy±chemotherapy; these patients received imneskibart+low-dose aldesleukin+avelumab at both the RP2D−1 and RP2D.

What is claimed is:

1. A combination therapy for treating unresectable locally advanced or metastatic non-small cell lung cancer (NSCLC) that is PD-L1 positive wherein treatment consists of 2^{nd} line or 3^{rd} line treatment, the combination therapy comprising an anti-IL-2 antibody or a pharmaceutical composition thereof, a loading dose of IL-2 or a pharmaceutical composition thereof, and avelumab or a pharmaceutical composition thereof,
wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5;
wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said avelumab is formulated for administration at a dose of 800 mg; and
wherein said pharmaceutical composition(s) further comprises a pharmaceutically acceptable carrier.

2. The combination therapy according to claim 1, wherein the amino acid sequence of the VH comprises the amino acid sequence of SEQ ID NO:6 and the amino acid sequence of VL comprises the amino acid sequence of SEQ ID NO:7.

3. The combination therapy according to claim 1, wherein the amino acid sequence of the full length heavy chain is set forth in SEQ ID NO:8 and the amino acid sequence of the full length light chain is set forth in SEQ ID NO:9.

4. The combination therapy according to claim 1, wherein the antibody comprises an IgG, IgA, IgM, IgE, IgD, a Fv, a scFv, a Fab, a F(ab′)₂, a minibody, a diabody, or a triabody.

5. The combination therapy according to claim 1, wherein said antibody comprises a heavy chain comprising a mutation that reduces binding to fragment crystallizable gamma receptors (FcγRs), wherein reduced binding is compared with said antibody lacking the mutation in the heavy chain that affects FcγRs binding.

6. The combination therapy according to claim 5, wherein said mutation comprises L234A, L235A (LALA) mutations.

7. A method of treating an unresectable locally advanced or metastatic non-small cell lung cancer (NSCLC) that is PD-L1 positive in a subject, said method comprising administering to said subject an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:1, said HCDR2 comprises the amino acid sequence of SEQ ID NO:2, said HCDR3 comprises the amino acid sequence of SEQ ID NO:3, said LCDR1 comprises the amino acid sequence of SEQ ID NO:4, said LCDR2 comprises the amino acid sequence of DAS, and said LCDR3 comprises the amino acid sequence of SEQ ID NO: 5;
wherein said NSCLC comprises unresectable locally advanced or metastatic NSCLC that is PD-L1 positive;
wherein said treating comprises second-line or third-line treatment;
wherein said pharmaceutical composition(s) further comprises a pharmaceutically acceptable carrier,
thereby treating said NSCLC in said subject.

8. The method according to claim 7, wherein the administration of said loading dose of IL-2 is prior to, concurrent with, or following the administration of said anti-IL-2 antibody, said avelumab, or both.

9. The method according to claim 7, further comprising the step of administering one or more additional doses of said anti-IL-2 antibody or a pharmaceutical composition thereof, wherein said pharmaceutical composition further comprises a pharmaceutically acceptable carrier.

10. The method according to claim 9, wherein said additional doses of said anti-IL-2 antibody or a pharmaceutical composition thereof are administered to said subject once every two weeks, wherein said pharmaceutical composition further comprises a pharmaceutically acceptable carrier.

11. The method according to claim 7, further comprising the step of administering one or more additional doses of said avelumab or a pharmaceutical composition thereof, wherein said pharmaceutical composition further comprises a pharmaceutically acceptable carrier.

12. The method according to claim 11, wherein said one or more additional doses of said avelumab or a pharmaceutical composition thereof are administered to said subject once every two weeks.

13. The method according to claim 7, further comprising the step of administering one or more booster doses of IL-2 or a pharmaceutical composition thereof, wherein said pharmaceutical composition further comprises a pharmaceutically acceptable carrier.

14. The method according to claim 13, wherein the administration of said one or more booster doses of IL-2 is prior to, concurrent with, or following the administration of said one or more additional doses of said anti-IL-2 antibody, one or more additional doses of said avelumab, or both.

15. The method according to claim 13, wherein at least one of the one or more IL-2 booster doses is administered at a dose of 135,000 IU/kg of said subject's body weight.

16. The method according to claim 13, wherein said at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

17. The method according to claim 7, wherein said method comprises (i) reducing the size of the tumor, (ii) inhibiting or reducing growth of the tumor, (iii) inhibiting or reducing metastases of said tumor, (iv) inhibiting the production of new lesions, (v) decreasing or shrinking target lesions, (vi) eliminating target lesions, or (vii) any combination thereof.

18. The method according to claim 7, wherein said NSCLC progressed after prior checkpoint inhibitor therapy in said subject.

19. The method according to claim 7 wherein said NSCLC comprises squamous NSCLC.

20. The method according to claim 7, wherein said NSCLC comprises non-squamous NSCLC.


SEQUENCE LISTING (10 sequences)

SEQ ID NO: 1 - protein, 7 aa, synthetic construct
GYSITDD

SEQ ID NO: 2 - protein, 8 aa, synthetic construct
IDPEDGET

SEQ ID NO: 3 - protein, 14 aa, synthetic construct
ARSLDSTWIYPFAY

SEQ ID NO: 4 - protein, 12 aa, synthetic construct
QSLLRRGNQKNH

SEQ ID NO: 5 - protein, 9 aa, synthetic construct
LQSYITPPT

SEQ ID NO: 6 - protein, 121 aa, synthetic construct
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSS

SEQ ID NO: 7 - protein, 113 aa, synthetic construct
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIK

SEQ ID NO: 8 - protein, 451 aa, synthetic construct
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 220 aa, synthetic construct
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 747 bp, synthetic construct
  source 1..747: mol_type = other DNA
caggtccaactggtgcagtccggtgccgaagttaaaaaacctgggtcttccgttaaagtttcttgcaaagcctctggctacagcatcaccgatgacctgattcactgggtccgtcaggctccaggtcaaggtctggaatggatgggttggatcgatccagaagacggtgaaaccaactatgcccagaaattccagggtcgtgtaaccctgaccgccgacacctccacctctaccgcctacatggagttaagtagcctgcgttcagaggataccgcagtgtactactgcgctcgttcactggactccacctggatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtcgcggtaatcagaaaaaccaccttgcatggtatcagcagaaaccaggtcagccaccaaaattactgatctatgacgcatctaccggtcaaagcggtgtcccagatcgtttcagcggttccggctccggtactgacttcaccctgaccatctcttcccttcaggccgaagatgtggccgtgtattactgcctgcagagctacatcaccccacctactttcggtgctggtactaaagttgaaatcaaa